SEQUENCE LISTING (46 sequences)

SEQ ID NO: 1 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 2 - DNA, 564 bp, Homo sapiens
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggctccgcagcctgaccaccctgctgcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 3 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGVALLSEAVLRGQALLVNSSQPWEPLQLHVDKAVSGVRSLTTLLRALGAQKEAISPPDAASAAPLRTITADTFRKLFRVYSNFLRGKLKLYTGEACRTGDR

SEQ ID NO: 4 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcgtggcgctgctgtccgaggccgtgctcaggggacaagcactcctcgtgaatagctcgcagccctgggaaccgctgcagctgcacgtggataaggctgtgtcaggggtccgcagcctgaccaccctgctgcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 5 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALVSEALLRGQALLVNSSQPWEPLQLHVDKAVSGIRSLTTLLRALGAQKEAISPPDAASAAPLRTITADTFRKLFRVYSNFLRGKLKLYTGEACRTGDR

SEQ ID NO: 6 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctggtgtccgaggccctgctcaggggacaagcactcctcgtgaatagctcgcagccctgggaaccgctgcagctgcacgtggataaggctgtgtcagggattcgcagcctgaccaccctgcttcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 7 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGTRSLTTLLRALGAQKEAISPPDAASAAPLRTITADTFRKLFRVYSNFLRGKLKLYTGEACRTGDR

SEQ ID NO: 8 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggacccgcagcctgaccaccctgctgcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 9 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGNALLSEANLRGQALLVNSSQPWEPLQLHVDKAVSGDRSLTTLLRALGAQKEAISPPDAASAAPLRTITADTFRKLFRVYSNFLRGKLKLYTGEACRTGDR

SEQ ID NO: 10 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcaacgcgctgctgtccgaggccaacctcaggggacaagcactcctcgtgaatagctcgcagccctgggaaccgctgcagctgcacgtggataaggctgtgtcaggggatcgcagcctgaccaccctgctgcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 11 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGDALLSEADLRGQALLVNSSQPWEPLQLHVDKAVSGNRSLTTLLRALGAQKEAISPPDAASAAPLRTITADTFRKLFRVYSNFLRGKLKLYTGEACRTGDR

SEQ ID NO: 12 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcgatgcgctgctgtccgaggccgacctcaggggacaagcactcctcgtgaatagctcgcagccctgggaaccgctgcagctgcacgtggataaggctgtgtcagggaaccgcagcctgaccaccctgctgcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 13 - protein, 166 aa, Artificial Sequence
  source: /note=“Description of Artificial Sequence Synthetic polypeptide”
APPRLICDSRVAERILLEAKEAENITTGCAEHCSLNENITVPDTKVNFYAWKRMEVGQQAVEVWQGLALLSEAVLRGQALLVNSSQPWEPLQLHVDKAVSGLRSFTTLLRALGAQKEAISPPDAASAAPLRTITADTFRKLFRVYSNFWRGKLKLYTGEACRTGDR

SEQ ID NO: 14 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtggcggaaagaatcctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggctccgcagcttcaccaccctgctgcgggccttgggcgcgcaaaaggaagccatctcccccccggacgccgccagcgcagcgccactgaggactattaccgccgacacattccgcaagctgtttcgggtgtactccaacttctggcggggaaagctgaagctgtacactggagaggcctgcagaacgggggaccgctaatga

SEQ ID NO: 15 - protein, 166 aa, Artificial Sequence
  source: /note=“Description of Artificial Sequence Synthetic polypeptide”
APPRLICDSRVIERWLLEAKEAENITTGCAEHCSLNENITVPDTKVNFYAWKRMEVGQQAVEVWQGLALLSEAVLRGQALLVNSSQPWEPLQLHVDKAVSGLRSSTTLLRALGAQKEAISPPDAASAAPLRTITADTFRKLFRVYSNFVRGKLKLYTGEACRTGDR

SEQ ID NO: 16 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgatcgaaagatggctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggctccgcagctccaccaccctgctgcgggccttgggcgcgcaaaaggaagccatctcccccccggacgccgccagcgcagcgccactgaggactattaccgccgacacattccgcaagctgtttcgggtgtactccaacttcgtgcggggaaagctgaagctgtacactggagaggcctgcagaacgggggaccgctaatga

SEQ ID NO: 17 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSFTTLLRALGAQKEAISPPDAASAAPLRTITADTFRKLFRVYSNFLRGKLKLYTGEACRTGDR

SEQ ID NO: 18 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggctccgcagcttcaccaccctgctgcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 19 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLATLLRALGAQKEAISPPDAASAAPLRTITADTFRKLFRVYSNFLRGKLKLYTGEACRTGDR

SEQ ID NO: 20 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggctccgcagcctggccaccctgctgcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 21 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLGTLLRALGAQKEAISPPDAASAAPLRTITADTFRKLFRVYSNFLRGKLKLYTGEACRTGDR

SEQ ID NO: 22 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggctccgcagcctgggcaccctgctgcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 23 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLHTLLRALGAQKEAISPPDAASAAPLRTITADTFRKLFRVYSNFLRGKLKLYTGEACRTGDR

SEQ ID NO: 24 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggctccgcagcctgcacaccctgctgcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 25 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLARALGAQKEAISPPDAASAAPLRTITADTFRKLFRVYSNFLRGKLKLYTGEACRTGDR

SEQ ID NO: 26 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggctccgcagcctgaccaccctggcgcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 27 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLWRALGAQKEAISPPDAASAAPLRTITADTFRKLFRVYSNFLRGKLKLYTGEACRTGDR

SEQ ID NO: 28 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggctccgcagcctgaccaccctgtggcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 29 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLCRALGCQKEAISPPDAASAAPLRTITADTFRKLFRVYSNFLRGKLKLYTGEACRTGDR

SEQ ID NO: 30 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggctccgcagcctgaccaccctgtgtcgggccttgggctgccaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 31 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGTRSSHTLWRALGAQKEAISPPDAASAAPLRTITADTFRKLFRVYSNFLRGKLKLYTGEACRTGDR

SEQ ID NO: 32 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggacccgcagctcccacaccctgtggcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 33 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGTRSSGTLWRALGAQKEAISPPDAASAAPLRTITADTFRKLFRVYSNFLRGKLKLYTGEACRTGDR

SEQ ID NO: 34 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggacccgcagctccggcaccctgtggcgggccttgggcgcgcaaaaggaagccatctcccccccggacgccgccagcgcagcgccactgaggactattaccgccgacacattccgcaagctgtttcgggtgtactccaacttcctccggggaaagctgaagctgtacactggagaggcctgcagaacgggggaccgctaatga

SEQ ID NO: 35 - protein, 166 aa, Artificial Sequence
  source: /note=“Description of Artificial Sequence Synthetic polypeptide”
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYNNFLRGQLKLYTGEACRTGDR

SEQ ID NO: 36 - DNA, 564 bp, Artificial Sequence
  source: /note=“Description of Artificial Sequence Synthetic polynucleotide”
atggaaaccgatactctgctgctctgggtccttctgctttgggtgcctggttcgactggagctccgcctcggttgatctgcgactcccgcgtgctggaaagatacctcctggaagccaaagaggccgagaacatcaccaccggttgtgccgagcattgctccctgaacgaaaacattaccgtgcccgacaccaaggtcaacttctacgcttggaagcggatggaagtcggccagcaggccgtggaggtctggcagggcctggcgctgctgtccgaggccgtgctcaggggacaagcactcctcgtgaatagctcgcagccctgggaaccgctgcagctgcacgtggataaggctgtgtcagggctccgcagcctgaccaccctgctgcgggccttgggcgcgcaaaaggaagccatctcccccccggacgccgccagcgcagcgccactgaggactattaccgccgacacattccgcaagctgtttcgggtgtacaacaacttcctccggggacagctgaagctgtacactggagaggcctgcagaacgggggaccgctaatga

SEQ ID NO: 37 - protein, 9 aa, Homo sapiens
VLRGQALLV

SEQ ID NO: 38 - protein, 9 aa, Homo sapiens
LRSLTTLLR

SEQ ID NO: 39 - protein, 9 aa, Homo sapiens
LLRALGAQK

SEQ ID NO: 40 - protein, 9 aa, Homo sapiens
FRVYSNFLR

SEQ ID NO: 41 - protein, 9 aa, Homo sapiens
YSNFLRGKL

SEQ ID NO: 42 - protein, 166 aa, Artificial Sequence; identical to SEQ ID NO: 1
  source: /note=“Description of Artificial Sequence Synthetic polypeptide”
  VARIANT (12)..(12): /replace=“A” or “I”
  VARIANT (15)..(15): /replace=“I” or “W”
  VARIANT (67)..(67): /replace=“D”, “N”, or “V”
  VARIANT (70)..(70): /replace=“V”
  VARIANT (74)..(74): /replace=“D”, “L”, or “N”
  VARIANT (102)..(102): /replace=“D”, “I”, “N”, “T”, or “V”
  VARIANT (105)..(105): /replace=“F” or “S”
  VARIANT (106)..(106): /replace=“A”, “G”, or “H”
  VARIANT (109)..(109): /replace=“A”, “C”, or “W”
  VARIANT (114)..(114): /replace=“C”
  VARIANT (146)..(146): /replace=“N”
  VARIANT (149)..(149): /replace=“V” or “W”
  VARIANT (152)..(152): /replace=“Q”
  SITE (1)..(166): /note=“Variant residues given in the sequence have no preference with respect to those in the annotations for variant positions”
  See specification as filed for detailed description of substitutions and preferred embodiments

SEQ ID NO: 43 - protein, 6 aa, Artificial Sequence
  source: /note=“Description of Artificial Sequence Synthetic 6xHis tag”
HHHHHH

SEQ ID NO: 44 - protein, 15 aa, Homo sapiens
LRSLTTLLRALGAQK

SEQ ID NO: 45 - protein, 12 aa, Homo sapiens
FRVYSNFLRGKL

SEQ ID NO: 46 - protein, 166 aa, Homo sapiens
APPRLICDSRVLERYLLEAKEAEKITTGCAEHCSLNEKITVPDTKVNFYAWKRMEVGQQAVEVWQGLALLSEAVLRGQALLVKSSQPWEPLQLHVDKAVSGLRSLTTLLRALGAQKEAISNSDAASAAPLRTITADTFRKLFRVYSNFLRGKLKLYTGEACRTGDR

(19) United States
(12) Patent Application Publication
(10) Pub. No.: US 2024/0109946 A1
(43) Pub. Date: Apr. 4, 2024

(54) ERYTHROPOIETIN VARIANTS AND USES THEREOF

(71) Applicant: Chulabhorn Foundation, Bangkok (TH)

(72) Inventors: Mathuros RUCHIRAWAT, Bangkok (TH); Mayuree FUANGTHONG, Bangkok (TH); Charlermchai ARTPRADIT, Bangkok (TH); Thanutsorn SUSANTAD, Bangkok (TH)

(21) Appl. No.: 18/037,505

(22) PCT Filed: Nov. 18, 2021
(86) PCT No.: PCT/IB2021/000786

§ 371 Date: May 17, 2023

Related U.S. Application Data
(60) Provisional application No. 63/115,208, filed on Nov. 18, 2020.

(51) Int. Cl. C07K 14/505; A61P 37/06; C12Q 1/6876

(52) U.S. Cl. CPC C07K 14/505; A61P 37/06; C12Q 1/6876; C12Q 2600/158; G01N 2800/52

(57) ABSTRACT

Human erythropoietin (EPO) variants are disclosed. The EPO variants comprise one or more mutations that reduce immunogenicity. The EPO variants can be used to treat or prevent disorders associated with EPO, for example, anemia.

### CROSS-REFERENCE TO RELATED APPLICATIONS

This application claims priority to U.S. Provisional Application No. 63/115,208, filed Nov. 18, 2020. The contents of the aforementioned application are incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 12, 2021, is named C2238-7000WO_SL.txt and is 51,498 bytes in size.

### BACKGROUND

Erythropoietin (EPO) is a protein hormone produced by the kidneys and plays an essential role in the production and maturation of red blood cells (RBCs), which carry oxygen from the lungs to the rest of the body. Recombinant human erythropoietin (rHuEPO) can be administered to subjects who have insufficient hemoglobin levels, for example, subjects with anemia and/or the inability to produce sufficient amounts of endogenous EPO, such as subjects with chronic kidney disease (CKD), HIV infection, or malignancy, and subjects dependent on dialysis. rHuEPO has also been used to accelerate erythropoiesis in surgery, post-chemotherapy and post-transplantation (Ng, T. et al. Postgrad. Med. J. (2003) 79:367-376). Treatment with rHuEPO has been shown to improve the quality of life (QoL) of subjects with anemia (Horl, et al. Nephrol. Dial. Transplant (1996) 11:246-250; Kaneko, Y. et al. Kidney Int. (2003) 63:1086-1093). However, despite advances in the treatment of anemia using rHuEPO, there exists an unmet need for developing new approaches, including new agents (e.g., EPO polypeptide variants), for treating and preventing anemia, including anemia associated with EPO deficiencies or related disorders.

### SUMMARY

In an aspect, the disclosure features an EPO molecule comprising a mutation at a position disclosed herein, e.g., having one or more functional properties disclosed herein.

In an embodiment, the EPO molecule comprises a mutation at a position corresponding to any of positions 12-152 of SEQ ID NO: 1. In an embodiment, the EPO molecule comprise a mutation at a position corresponding to any of positions 74-82, 102-116, or 142-153 of SEQ ID NO: 1. In an embodiment, the EPO molecule comprises a mutation at a position corresponding to any of positions 74-82 or 102-116 of SEQ ID NO: 1. In an embodiment, the EPO molecule comprises a mutation at a position corresponding to any of positions 74-82 or 142-153 of SEQ ID NO: 1. In an embodiment, the EPO molecule comprises a mutation at a position corresponding to any of positions 102-116 or 142-153 of SEQ ID NO: 1. In an embodiment, the EPO molecule comprises a mutation at a position corresponding to any of positions 74-82 of SEQ ID NO: 1. In an embodiment, the EPO molecule comprises a mutation at a position corresponding to any of positions 102-116 of SEQ ID NO: 1. In an embodiment, the EPO molecule comprises a mutation at a position corresponding to any of positions 102-110 of SEQ ID NO: 1. In an embodiment, the EPO molecule comprises a mutation at a position corresponding to any of positions 108-116 of SEQ ID NO: 1. In an embodiment, the EPO molecule comprises a mutation at a position corresponding to any of positions 142-153 of SEQ ID NO: 1. In an embodiment, the EPO molecule comprises a mutation at a position corresponding to any of positions 142-150 of SEQ ID NO: 1. In an embodiment, the EPO molecule comprises a mutation at a position corresponding to any of positions 145-153 of SEQ ID NO: 1. In an embodiment, the EPO molecule comprises a mutation at a position corresponding to any of positions 74, 75, 76, 77, 79, 80, 81, 82, 102, 105, 106, 109, 111, 112, 113, 114, 115, 116, 142, 145, 146, 148, 149, 152, or 153 of SEQ ID NO: 1. In an embodiment, the EPO molecule comprises a mutation at a position corresponding to any of positions 12, 15, 67, 70, 74, 102, 105, 106, 109, 114, 146, 149, or 152 of SEQ ID NO: 1.

In an embodiment, the EPO molecule comprises a plurality of mutations (e.g., a plurality of mutations disclosed herein) at a plurality of positions (e.g., a plurality of mutations disclosed herein). In an embodiment, the EPO molecule further comprises a second mutation at a second position disclosed herein. In an embodiment, the EPO molecule further comprises a third mutation at a third position disclosed herein. In an embodiment, the EPO molecule further comprises a fourth mutation at a fourth position disclosed herein. In an embodiment, the EPO molecule further comprises a fifth mutation at a fifth position disclosed herein. In an embodiment, the EPO molecule further comprises a sixth mutation at a sixth position disclosed herein.

In an embodiment, the EPO molecule comprises a mutation at a position corresponding to position 12 of SEQ ID NO: 1. In an embodiment, the mutation is an L12A substitution. In an embodiment, the mutation is an L12I substitution.

In an embodiment, the EPO molecule comprises a mutation at a position corresponding to position 15 of SEQ ID NO: 1. In an embodiment, the mutation is a Y15I substitution. In an embodiment, the mutation is a Y15W substitution.

In an embodiment, the EPO molecule comprises a mutation at a position corresponding to position 67 of SEQ ID NO: 1. In an embodiment, the mutation is a L67D substitution. In an embodiment, the mutation is a L67N substitution. In an embodiment, the mutation is a L67V substitution.

In an embodiment, the EPO molecule comprises a mutation at a position corresponding to position 70 of SEQ ID NO: 1. In an embodiment, the mutation is an L70V substitution.

In an embodiment, the EPO molecule comprises a mutation at a position corresponding to position 74 of SEQ ID NO: 1. In an embodiment, the mutation is a V74D substitution. In an embodiment, the mutation is a V74L substitution. In an embodiment, the mutation is a V74N substitution.

In an embodiment, the EPO molecule comprises a mutation at a position corresponding to position 102 of SEQ ID NO: 1. In an embodiment, the mutation is an L102D substitution. In an embodiment, the mutation is an L102N substitution. In an embodiment, the mutation is an L1021 substitution. In an embodiment, the mutation is an L102T substitution. In an embodiment, the mutation is an L102V substitution.

In an embodiment, the EPO molecule comprises a mutation at a position corresponding to position 105 of SEQ ID NO: 1. In an embodiment, the mutation is an L105F substitution. In an embodiment, the mutation is an L105S substitution.

In an embodiment, the EPO molecule comprises a mutation at a position corresponding to position 106 of SEQ ID NO: 1. In an embodiment, the mutation is a T106A substitution. In an embodiment, the mutation is a T106G substitution. In an embodiment, the mutation is a T106H substitution.

In an embodiment, the EPO molecule comprises a mutation at a position corresponding to position 109 of SEQ ID NO: 1. In an embodiment, the mutation is an L109A substitution. In an embodiment, the mutation is an L109C substitution. In an embodiment, the mutation is an L109W substitution.

In an embodiment, the EPO molecule comprises a mutation at a position corresponding to position 114 of SEQ ID NO: 1. In an embodiment, the mutation is an A114C substitution.

In an embodiment, the EPO molecule comprises a mutation at a position corresponding to position 146 of SEQ ID NO: 1. In an embodiment, the mutation is an S146N substitution.

In an embodiment, the EPO molecule comprises a mutation at a position corresponding to position 149 of SEQ ID NO: 1. In an embodiment, the mutation is an L149V substitution. In an embodiment, the mutation is an L149W substitution.

In an embodiment, the EPO molecule comprises a mutation at a position corresponding to position 152 of SEQ ID NO: 1. In an embodiment, the mutation is a K152Q substitution.

In an embodiment, the EPO molecule comprises mutations at positions corresponding to positions 67 and 102 of SEQ ID NO: 1. In an embodiment, the mutations are L67V and L102V substitutions. In an embodiment, the EPO molecule comprises mutations at positions corresponding to positions 70, 74, and 102.

In an embodiment, the mutations are L70V, V74L, and L1021 substitutions.

In an embodiment, the EPO molecule comprises mutations at positions corresponding to positions 67, 74, and 102. In an embodiment, the mutations are L67N, V74N, and L102D substitutions. In an embodiment, the mutations are L67D, V74D, and L102N substitutions.

In an embodiment, the EPO molecule comprises mutations at positions corresponding to positions 12, 15, 105, and 149. In an embodiment, the mutations are L12A, Y15L L105F, and L149W substitutions. In an embodiment, the mutations are L12L Y15W, L105S, and L149V substitutions. In an embodiment, the EPO molecule comprises mutations at positions corresponding to positions 109 and 114. In an embodiment, the mutations are L109C and A114C substitutions.

In an embodiment, the EPO molecule comprises mutations at positions corresponding to positions 102, 105, 106, and 109. In an embodiment, the mutations are L102T, L105S, T106H, and L109W substitutions. In an embodiment, the mutations are L102T, L105S, T106G, and L109W substitutions.

In an embodiment, the EPO molecule comprises mutations at positions corresponding to positions 146 and 152. In an embodiment, the mutations are S146N and K152Q substitutions.

In an embodiment, the EPO molecule has at least 80% homology with the amino acid sequence of SEQ ID NO: 1. In an embodiment, the EPO molecule has at least 85% homology with the amino acid sequence of SEQ ID NO: 1. In an embodiment, the EPO molecule has at least 90% homology with the amino acid sequence of SEQ ID NO: 1. In an embodiment, the EPO molecule has at least 95% homology with the amino acid sequence of SEQ ID NO: 1. In an embodiment, the EPO molecule has at least 96% homology with the amino acid sequence of SEQ ID NO: 1. In an embodiment, the EPO molecule has at least 97% homology with the amino acid sequence of SEQ ID NO: 1. In an embodiment, the EPO molecule has at least 98% homology with the amino acid sequence of SEQ ID NO: 1. In an embodiment, the EPO molecule has at least 99% homology with the amino acid sequence of SEQ ID NO: 1.

In an embodiment, the EPO molecule, other than the mutation(s) at the position(s) disclosed herein, differs at no more than 15 positions with the amino acid sequence of SEQ ID NO: 1. In an embodiment, the EPO molecule, other than the mutation(s) at the position(s) disclosed herein, differs at no more than 10 positions with the amino acid sequence of SEQ ID NO: 1. In an embodiment, the EPO molecule, other than the mutation(s) at the position(s) disclosed herein, differs at no more than 5 positions with the amino acid sequence of SEQ ID NO: 1. In an embodiment, the EPO molecule, other than the mutation(s) at the position(s) disclosed herein, differs at no more than 4 positions with the amino acid sequence of SEQ ID NO: 1. In an embodiment, the EPO molecule, other than the mutation(s) at the position(s) disclosed herein, differs at no more than 3 positions with the amino acid sequence of SEQ ID NO: 1. In an embodiment, the EPO molecule, other than the mutation(s) at the position(s) disclosed herein, differs at no more than 2 positions with the amino acid sequence of SEQ ID NO: 1. In an embodiment, the EPO molecule, other than the mutation(s) at the position(s) disclosed herein, differs at no more than 1 position with the amino acid sequence of SEQ ID NO: 1.

In an embodiment, the EPO molecule differs at no more than 15 positions with the amino acid sequence of SEQ ID NO: 1. In an embodiment, the EPO molecule differs at no more than 10 positions with the amino acid sequence of SEQ ID NO: 1. In an embodiment, the EPO molecule differs at no more than 5 positions with the amino acid sequence of SEQ ID NO: 1. In an embodiment, the EPO molecule differs at no more than 4 positions with the amino acid sequence of SEQ ID NO: 1. In an embodiment, the EPO molecule differs at no more than 3 positions with the amino acid sequence of SEQ ID NO: 1. In an embodiment, the EPO molecule differs at no more than 2 positions with the amino acid sequence of SEQ ID NO: 1. In an embodiment, the EPO molecule differs at no more than 1 position with the amino acid sequence of SEQ ID NO: 1.

In an embodiment, the EPO molecule comprises the amino acid sequence of any of SEQ ID NOs: 3, 5, 7, 9, 11, 13, 15, 17, 19, 21, 23, 25, 27, 29, 31, 33, or 35, or an amino acid sequence that is at least 90%, 91%, 92%, 93%, 94%, 95%, 96%, 97%, 98%, or 99% identical thereto, or differs by no more than 1, 2, 3, 4, 5, 6, 7. 8, 9, or 10 amino acids therefrom.

In an embodiment, the EPO molecule comprises the amino acid sequence of any of SEQ ID NOs: 3, 5, 7, 9, 11, 13, 15, 17, 19, 21, 23, 25, 27, 29, 31, 33, or 35.

In an embodiment, the EPO molecule comprises or consists of the amino acid sequence of SEQ ID NO: 3. In an embodiment, the EPO molecule comprises or consists of the amino acid sequence of SEQ ID NO: 5. In an embodiment, the EPO molecule comprises or consists of the amino acid sequence of SEQ ID NO: 7. In an embodiment, the EPO molecule comprises or consists of the amino acid sequence of SEQ ID NO: 9. In an embodiment, the EPO molecule comprises or consists of the amino acid sequence of SEQ ID NO: 11. In an embodiment, the EPO molecule comprises or consists of the amino acid sequence of SEQ ID NO: 13. In an embodiment, the EPO molecule comprises or consists of the amino acid sequence of SEQ ID NO: 15. In an embodiment, the EPO molecule comprises or consists of the amino acid sequence of SEQ ID NO: 17. In an embodiment, the EPO molecule comprises or consists of the amino acid sequence of SEQ ID NO: 19. In an embodiment, the EPO molecule comprises or consists of the amino acid sequence of SEQ ID NO: 21. In an embodiment, the EPO molecule comprises or consists of the amino acid sequence of SEQ ID NO: 23. In an embodiment, the EPO molecule comprises or consists of the amino acid sequence of SEQ ID NO: 25. In an embodiment, the EPO molecule comprises or consists of the amino acid sequence of SEQ ID NO: 27. In an embodiment, the EPO molecule comprises or consists of the amino acid sequence of SEQ ID NO: 29. In an embodiment, the EPO molecule comprises or consists of the amino acid sequence of SEQ ID NO: 31. In an embodiment, the EPO molecule comprises or consists of the amino acid sequence of SEQ ID NO: 33. In an embodiment, the EPO molecule comprises or consists of the amino acid sequence of SEQ ID NO: 35.

In an embodiment, the EPO molecule has a reduced immunogenicity in a subject, as compared to a reference EPO molecule, e.g., an EPO molecule comprising the amino acid sequence of SEQ ID NO: 1, e.g., as determined in vitro, ex vivo, or in vivo. In an embodiment, the immunogenicity is reduced by at least 10%, 20%, 30%, 40%, 50%, 60%, 70%, 80%, or 90%, as compared to the reference EPO molecule. In an embodiment, the subject carries the HLA-DRB1*09 allele.

In an embodiment, the EPO molecule has the same, or substantially the same, biological activity, as compared to a reference EPO molecule, e.g., an EPO molecule comprising the amino acid sequence of SEQ ID NO: 1, e.g., as determined in vitro, ex vivo, or in vivo. In an embodiment, the EPO molecule has a biological activity that is reduced by no more than 25%, 20%, 15%, 10%, 5%, or 2%, as compared to a reference EPO molecule, e.g., an EPO molecule comprising the amino acid sequence of SEQ ID NO: 1. In an embodiment, the biological activity is determined in an in vitro TF-1 proliferation assay.

In an embodiment, the EPO molecule is capable of having more glycoforms, e.g., at least 1, 2, 3, 4, or 5 more glycoforms, as compared to a reference EPO molecule, e.g., an EPO molecule comprising the amino acid sequence of SEQ ID NO: 1. In an embodiment, the EPO molecule is capable of having 6, 7, 8, 9, 10, or more glycoforms. In an embodiment, the EPO molecule is capable of having 7, 8, or more glycoforms.

In an embodiment, the EPO molecule has the same, or substantially the same, serum half-life, as compared to a reference EPO molecule, e.g., an EPO molecule comprising the amino acid sequence of SEQ ID NO: 1. In an embodiment, the EPO molecule has an increased serum half-life, e.g., increased by at least 10%, 20%, 30%, 40%, 50%, 60%, 70%, 80%, or 90%, or at least 1, 2, 3, 4, 5, 6, 7, 8, 9, or 10-fold, as compared to a reference EPO molecule, e.g., an EPO molecule comprising the amino acid sequence of SEQ ID NO: 1. In an embodiment, the EPO molecule has a reduced serum half-life, e.g., reduced by no more than 10%, 20%, 30%, 40%, 50%, 60%, 70%, 80%, or 90%, as compared to a reference EPO molecule, e.g., an EPO molecule comprising the amino acid sequence of SEQ ID NO: 1.

In an embodiment, the EPO molecule comprises a moiety that extends the serum half-life of an EPO polypeptide. In an embodiment, the EPO molecule is a fusion protein. In an embodiment, the EPO molecule comprises an Fc domain of an IgG. In an embodiment, the Fc domain covalently fused to an EPO polypeptide. In an embodiment, the N-terminus of the Fc domain is covalently fused to the C-terminus of the EPO polypeptide directly or indirectly.

In an embodiment, the EPO molecule is an isolated EPO molecule. In an embodiment, the EPO molecule is a recombinant EPO molecule. In an embodiment, the EPO molecule is a synthetic EPO molecule.

In an aspect, the disclosure features a pharmaceutical composition comprising an EPO molecule disclosed herein and a pharmaceutically acceptable carrier.

In an aspect, the disclosure features a preparation comprising an EPO molecule disclosed herein. In an embodiment, the preparation is suitable for therapeutic use.

In an aspect, the disclosure features a kit comprising an EPO molecule disclosed herein and instructions for use.

In an aspect, the disclosure features a nucleic acid encoding an EPO molecule disclosed herein.

In an aspect, the disclosure features a nucleic acid comprising the nucleotide sequence of any of SEQ ID NOs: 4, 6, 8, 10, 12, 14, 16, 18, 20, 22, 24, 26, 28, 30, 32, 34, or 36, or a nucleotide sequence that differs by no more than 5, 10, 15, 20, 25, 30, 35, 40, 45, or 50 nucleotides therefrom, or is at least 80%, 85%, 90%, 95%, 98%, or 99% identical thereto. In an embodiment, the nucleic acid comprises the nucleotide sequence of any of SEQ ID NOs: 4, 6, 8, 10, 12, 14, 16, 18, 20, 22, 24, 26, 28, 30, 32, 34, or 36.

In an aspect, the disclosure features a vector comprising a nucleic acid disclosed herein. In an embodiment, the vector comprises a promoter operably linked to the nucleic acid.

In an aspect, the disclosure features a cell comprising a nucleic acid disclosed herein or a vector disclosed herein.

In an aspect, the disclosure features a method of producing an EPO molecule, comprising culturing a cell disclosed herein under conditions that allows for expression of EPO polypeptide, thereby producing the EPO molecule.

In an aspect, the disclosure features a method of treating a disorder in a subject, comprising administering to the subject an effective amount of an EPO molecule disclosed herein, thereby treating the disorder.

In an embodiment, the disorder is a disorder associated with EPO, e.g., a deficiency or abnormality of EPO. In an embodiment, the disorder is anemia. In an embodiment, the anemia is associated with or due to a chronic kidney disease (e.g., chronic renal failure), a zidovudine treatment, a chemotherapy, or a surgery.

In an embodiment, the method further comprises acquiring genotype information that identifies an HLA genotype, e.g., the HLA-DRB1*09 genotype, in the subject, wherein the presence of the HLA genotype identifies the subject has, or is at risk of having, an increased immunogenicity to the EPO therapy. In an embodiment, the EPO molecule is administered responsive to the acquisition of the genotype information.

In an embodiment, the subject carries, or has been determined to carry, the HLA-DRB1*09 allele. In an embodiment, the subject has a chronic kidney disease, an HIV infection, or a cancer. In an embodiment, the subject receives, or has received, zidovudine. In an embodiment, the subject receives, or has received, a chemotherapy (e.g., a myelosuppressive chemotherapy), a surgery, or an allogenic RBC transfusion. In an embodiment, the subject has, or has been determined to have, anti-EPO antibodies. In an embodiment, the subject has been administered a different recombinant human EPO (rHuEPO), e.g., an EPO comprising the amino acid sequence of SEQ ID NO: 1. In an embodiment, the administration of the different rHuEPO is discontinued or terminated.

In an aspect, the disclosure features a method of reducing an immunogenic response in a subject, comprising administering to a subject in need thereof an effective amount of an EPO molecule disclosed herein, thereby reducing the immunogenic response.

In an embodiment, the method further comprises acquiring genotype information that identifies an HLA genotype, e.g., the HLA-DRB1*09 genotype, in the subject, wherein the presence of the HLA genotype identifies the subject has, or is at risk of having, an increased immunogenicity to the EPO therapy. In an embodiment, the EPO molecule is administered responsive to the acquisition of the genotype information.

In an embodiment, the subject carries, or has been determined to carry, the HLA-DRB1*09 allele. In an embodiment, the subject has a chronic kidney disease, an HIV infection, or a cancer. In an embodiment, the subject receives, or has received, zidovudine. In an embodiment, the subject receives, or has received, a chemotherapy (e.g., a myelosuppressive chemotherapy), a surgery, or an allogenic RBC transfusion. In an embodiment, the subject has, or has been determined to have, anti-EPO antibodies. In an embodiment, the subject has been administered a different recombinant human EPO (rHuEPO), e.g., an EPO comprising the amino acid sequence of SEQ ID NO: 1. In an embodiment, the administration of the different rHuEPO is discontinued or terminated.

In an aspect, the disclosure features a method of treating or preventing pure red-cell aplasia (PRCA) in a subject, comprising administering to a subject in need thereof an effective amount of an EPO molecule disclosed herein, thereby treating or preventing PRCA.

In an embodiment, the method further comprises acquiring genotype information that identifies an HLA genotype, e.g., the HLA-DRB1*09 genotype, in the subject, wherein the presence of the HLA genotype identifies the subject has, or is at risk of having, an increased immunogenicity to the EPO therapy. In an embodiment, the EPO molecule is administered responsive to the acquisition of the genotype information.

In an embodiment, the subject carries, or has been determined to carry, the HLA-DRB1*09 allele. In an embodiment, the subject has a chronic kidney disease, an HIV infection, or a cancer. In an embodiment, the subject receives, or has received, zidovudine. In an embodiment, the subject receives, or has received, a chemotherapy (e.g., a myelosuppressive chemotherapy), a surgery, or an allogenic RBC transfusion. In an embodiment, the subject has, or has been determined to have, anti-EPO antibodies. In an embodiment, the subject has been administered a different recombinant human EPO (rHuEPO), e.g., an EPO comprising the amino acid sequence of SEQ ID NO: 1. In an embodiment, the administration of the different rHuEPO is discontinued or terminated.

In an aspect, the disclosure features a method of reducing an allogenic red blood cell (RBC) transfusion in a subject, comprising administering to a subject in need thereof an effective amount of an EPO molecule disclosed herein, thereby reducing the allogenic RBC transfusion.

In an embodiment, the method further comprises acquiring genotype information that identifies an HLA genotype, e.g., the HLA-DRB1*09 genotype, in the subject, wherein the presence of the HLA genotype identifies the subject has, or is at risk of having, an increased immunogenicity to the EPO therapy. In an embodiment, the EPO molecule is administered responsive to the acquisition of the genotype information.

In an embodiment, the subject carries, or has been determined to carry, the HLA-DRB1*09 allele. In an embodiment, the subject has a chronic kidney disease, an HIV infection, or a cancer. In an embodiment, the subject receives, or has received, zidovudine. In an embodiment, the subject receives, or has received, a chemotherapy (e.g., a myelosuppressive chemotherapy), a surgery, or an allogenic RBC transfusion. In an embodiment, the subject has, or has been determined to have, anti-EPO antibodies. In an embodiment, the subject has been administered a different recombinant human EPO (rHuEPO), e.g., an EPO comprising the amino acid sequence of SEQ ID NO: 1. In an embodiment, the administration of the different rHuEPO is discontinued or terminated.

In an aspect, the disclosure features a method of evaluating a subject, comprising acquiring genotype information that identifies an HLA genotype, e.g., the HLA-DRB1*09 genotype, in the subject, wherein the presence of the HLA genotype identifies the subject has, or is at risk of having, an increased immunogenicity to a therapy comprising an EPO molecule.

In an embodiment, the method further comprises classifying the subject as a candidate for treatment with an EPO molecule disclosed herein. In an embodiment, the method further comprises administering to the subject an effective amount of an EPO molecule disclosed herein.

In an embodiment, the subject carries, or has been determined to carry, the HLA-DRB1*09 allele. In an embodiment, the subject has a chronic kidney disease, an HIV infection, or a cancer. In an embodiment, the subject receives, or has received, zidovudine. In an embodiment, the subject receives, or has received, a chemotherapy (e.g., a myelosuppressive chemotherapy), a surgery, or an allogenic RBC transfusion. In an embodiment, the subject has, or has been determined to have, anti-EPO antibodies. In an embodiment, the subject has been administered a different recombinant human EPO (rHuEPO), e.g., an EPO comprising the amino acid sequence of SEQ ID NO: 1. In an embodiment, the administration of the different rHuEPO is discontinued or terminated.

In an aspect, the disclosure features an EPO molecule described herein for use as a medicament.

In an aspect, the disclosure features an EPO molecule described herein for use in treating a disorder in a subject (e.g., a subject described herein) in accordance with a method described herein.

In an aspect, the disclosure features an EPO molecule described herein for use in reducing an immunogenic response in a subject (e.g., a subject described herein) in accordance with a method described herein.

In an aspect, the disclosure features an EPO molecule described herein for use in treating or preventing pure red-cell aplasia (PRCA) in a subject (e.g., a subject described herein) in accordance with a method described herein.

In an aspect, the disclosure features an EPO molecule described herein for use in reducing an allogenic red blood cell (RBC) transfusion in a subject (e.g., a subject described herein) in accordance with a method described herein.

In an aspect, the disclosure features use of an EPO molecule described herein in the manufacture of a medicament for treating a disorder in a subject (e.g., a subject described herein) in accordance with a method described herein.

In an aspect, the disclosure features use of an EPO molecule described herein in the manufacture of a medicament for reducing an immunogenic response in a subject (e.g., a subject described herein) in accordance with a method described herein.

In an aspect, the disclosure features use of an EPO molecule described herein in the manufacture of a medicament for treating or preventing pure red-cell aplasia (PRCA) in a subject (e.g., a subject described herein) in accordance with a method described herein.

In an aspect, the disclosure features use of an EPO molecule described herein in the manufacture of a medicament for reducing an allogenic red blood cell (RBC) transfusion in a subject (e.g., a subject described herein) in accordance with a method described herein.

### Enumerated Embodiments

1. An EPO molecule comprising a mutation at a position disclosed herein.
2. The EPO molecule of embodiment 1, comprising a mutation at a position corresponding to any of positions 12-152 of SEQ ID NO: 1.
3. The EPO molecule of embodiment 1 or 2, comprising a mutation at a position corresponding to any of positions 74-82, 102-116, or 142-153 of SEQ ID NO: 1.
4. The EPO molecule of any of embodiments 1-3, comprising a mutation at a position corresponding to any of positions 74-82 or 102-116 of SEQ ID NO: 1.
5. The EPO molecule of any of embodiments 1-4, comprising a mutation at a position corresponding to any of positions 74-82 or 142-153 of SEQ ID NO: 1.
6. The EPO molecule of any of embodiments 1-5, comprising a mutation at a position corresponding to any of positions 102-116 or 142-153 of SEQ ID NO: 1.
7. The EPO molecule of any of embodiments 1-6, comprising a mutation at a position corresponding to any of positions 74-82 of SEQ ID NO: 1.
8. The EPO molecule of any of embodiments 1-7, comprising a mutation at a position corresponding to any of positions 102-116 of SEQ ID NO: 1.
9. The EPO molecule of any of embodiments 1-8, comprising a mutation at a position corresponding to any of positions 102-110 of SEQ ID NO: 1.
10. The EPO molecule of any of embodiments 1-9, comprising a mutation at a position corresponding to any of positions 108-116 of SEQ ID NO: 1.
11. The EPO molecule of any of embodiments 1-10, comprising a mutation at a position corresponding to any of positions 142-153 of SEQ ID NO: 1.
12. The EPO molecule of any of embodiments 1-11, comprising a mutation at a position corresponding to any of positions 142-150 of SEQ ID NO: 1.
13. The EPO molecule of any of embodiments 1-12, comprising a mutation at a position corresponding to any of positions 145-153 of SEQ ID NO: 1.
14. The EPO molecule of any of embodiments 1-13, comprising a mutation at a position corresponding to any of positions 74, 75, 76, 77, 79, 80, 81, 82, 102, 105, 106, 109, 111, 112, 113, 114, 115, 116, 142, 145, 146, 148, 149, 152, or 153 of SEQ ID NO: 1.
15. The EPO molecule of any of embodiments 1-14, comprising a mutation at a position corresponding to any of positions 12, 15, 67, 70, 74, 102, 105, 106, 109, 114, 146, 149, or 152 of SEQ ID NO: 1.
16. The EPO molecule of any of embodiments 1-15, comprising a plurality of mutations (e.g., a plurality of mutations disclosed herein) at a plurality of positions (e.g., a plurality of positions disclosed herein).
17. The EPO molecule of embodiment 16, further comprising a second mutation at a second position disclosed herein.
18. The EPO molecule of embodiment 17, further comprising a third mutation at a third position disclosed herein.
19. The EPO molecule of embodiment 18, further comprising a fourth mutation at a fourth position disclosed herein.
20. The EPO molecule of embodiment 19, further comprising a fifth mutation at a fifth position disclosed herein.
21. The EPO molecule of any of embodiments 1-20, comprising a mutation at a position corresponding to position 12 of SEQ ID NO: 1.
22. The EPO molecule of embodiment 21, wherein the mutation is an L12A substitution.
23. The EPO molecule of embodiment 21, wherein the mutation is an L12I substitution.
24. The EPO molecule of any of embodiments 1-23, comprising a mutation at a position corresponding to position 15 of SEQ ID NO: 1.
25. The EPO molecule of embodiment 24, wherein the mutation is a Y15I substitution.
26. The EPO molecule of embodiment 24, wherein the mutation is a Y15W substitution.
27. The EPO molecule of any of embodiments 1-26, comprising a mutation at a position corresponding to position 67 of SEQ ID NO: 1.
28. The EPO molecule of embodiment 27, wherein the mutation is a L67D substitution.
29. The EPO molecule of embodiment 27, wherein the mutation is a L67N substitution.
30. The EPO molecule of embodiment 27, wherein the mutation is a L67V substitution.
31. The EPO molecule of any of embodiments 1-30, comprising a mutation at a position corresponding to position 70 of SEQ ID NO: 1.
32. The EPO molecule of embodiment 31, wherein the mutation is an L70V substitution.
33. The EPO molecule of any of embodiments 1-32, comprising a mutation at a position corresponding to position 74 of SEQ ID NO: 1.
34. The EPO molecule of embodiment 33, wherein the mutation is a V74D substitution.
35. The EPO molecule of embodiment 33, wherein the mutation is a V74L substitution.
36. The EPO molecule of embodiment 33, wherein the mutation is a V74N substitution.
37. The EPO molecule of any of embodiments 1-36, comprising a mutation at a position corresponding to position 102 of SEQ ID NO: 1.
38. The EPO molecule of embodiment 37, wherein the mutation is an L102D substitution.
39. The EPO molecule of embodiment 37, wherein the mutation is an L102N substitution.
40. The EPO molecule of embodiment 37, wherein the mutation is an L1021 substitution.
41. The EPO molecule of embodiment 37, wherein the mutation is an L102T substitution.
42. The EPO molecule of embodiment 37, wherein the mutation is an L102V substitution.
43. The EPO molecule of any of embodiments 1-42, comprising a mutation at a position corresponding to position 105 of SEQ ID NO: 1.
44. The EPO molecule of embodiment 43, wherein the mutation is an L105F substitution.
45. The EPO molecule of embodiment 43, wherein the mutation is an L105S substitution.
46. The EPO molecule of any of embodiments 1-45, comprising a mutation at a position corresponding to position 106 of SEQ ID NO: 1.
47. The EPO molecule of embodiment 46, wherein the mutation is a T106A substitution.
48. The EPO molecule of embodiment 46, wherein the mutation is a T106G substitution.
49. The EPO molecule of embodiment 46, wherein the mutation is a T106H substitution.
50. The EPO molecule of any of embodiments 1-49, comprising a mutation at a position corresponding to position 109 of SEQ ID NO: 1.
51. The EPO molecule of embodiment 50, wherein the mutation is an L109A substitution.
52. The EPO molecule of embodiment 50, wherein the mutation is an L109C substitution.
53. The EPO molecule of embodiment 50, wherein the mutation is an L109W substitution.
54. The EPO molecule of any of embodiments 1-53, comprising a mutation at a position corresponding to position 114 of SEQ ID NO: 1.
55. The EPO molecule of embodiment 54, wherein the mutation is an A114C substitution.
56. The EPO molecule of any of embodiments 1-55, comprising a mutation at a position corresponding to position 146 of SEQ ID NO: 1.
57. The EPO molecule of embodiment 56, wherein the mutation is an S146N substitution.
58. The EPO molecule of any of embodiments 1-57, comprising a mutation at a position corresponding to position 149 of SEQ ID NO: 1.
59. The EPO molecule of embodiment 58, wherein the mutation is an L149V substitution.
60. The EPO molecule of embodiment 58, wherein the mutation is an L149W substitution.
61. The EPO molecule of any of embodiments 1-60, comprising a mutation at a position corresponding to position 152 of SEQ ID NO: 1.
62. The EPO molecule of embodiment 61, wherein the mutation is a K152Q substitution.
63. The EPO molecule of any of embodiments 1-62, comprising mutations at positions corresponding to positions 67 and 102 of SEQ ID NO: 1.
64. The EPO molecule of embodiment 63, wherein the mutations are L67V and L102V substitutions.
65. The EPO molecule of any of embodiments 1-64, comprising mutations at positions corresponding to positions 70, 74, and 102.
66. The EPO molecule of embodiment 65, wherein the mutations are L70V, V74L, and L1021 substitutions.
67. The EPO molecule of any of embodiments 1-66, comprising mutations at positions corresponding to positions 67, 74, and 102.
68. The EPO molecule of embodiment 67, wherein the mutations are L67N, V74N, and L102D substitutions.
69. The EPO molecule of embodiment 67, wherein the mutations are L67D, V74D, and L102N substitutions.
70. The EPO molecule of any of embodiments 1-69, comprising mutations at positions corresponding to positions 12, 15, 105, and 149.
71. The EPO molecule of embodiment 70, wherein the mutations are L12A, Y151, L105F, and L149W substitutions.
72. The EPO molecule of embodiment 70, wherein the mutations are L12L Y15W, L105S, and L149V substitutions.
73. The EPO molecule of any of embodiments 1-72, comprising mutations at positions corresponding to positions 109 and 114.
74. The EPO molecule of embodiment 73, wherein the mutations are L109C and A114C substitutions.
75. The EPO molecule of any of embodiments 1-74, comprising mutations at positions corresponding to positions 102, 105, 106, and 109.
76. The EPO molecule of embodiment 75, wherein the mutations are L102T, L105S, T106H, and L109W substitutions.
77. The EPO molecule of embodiment 75, wherein the mutations are L102T, L105S, T106G, and L109W substitutions.
78. The EPO molecule of any of embodiments 1-77, comprising mutations at positions corresponding to positions 146 and 152.
79. The EPO molecule of embodiment 78, wherein the mutations are S146N and K152Q substitutions.
80. The EPO molecule of any of embodiments 1-79, having at least 80% homology with the amino acid sequence of SEQ ID NO: 1.
81. The EPO molecule of any of embodiments 1-80, having at least 85% homology with the amino acid sequence of SEQ ID NO: 1.
82. The EPO molecule of any of embodiments 1-81, having at least 90% homology with the amino acid sequence of SEQ ID NO: 1.
83. The EPO molecule of any of embodiments 1-82, having at least 95% homology with the amino acid sequence of SEQ ID NO: 1.
84. The EPO molecule of any of embodiments 1-83, having at least 96% homology with the amino acid sequence of SEQ ID NO: 1.
85. The EPO molecule of any of embodiments 1-84, having at least 97% homology with the amino acid sequence of SEQ ID NO: 1.
86. The EPO molecule of any of embodiments 1-85, having at least 98% homology with the amino acid sequence of SEQ ID NO: 1.
87. The EPO molecule of any of embodiments 1-86, having at least 99% homology with the amino acid sequence of SEQ ID NO: 1.
88. The EPO molecule of any of embodiments 1-87, which, other than the mutation(s) at the position(s) disclosed herein, differs at no more than 15 positions with the amino acid sequence of SEQ ID NO: 1.
89. The EPO molecule of any of embodiments 1-88, which, other than the mutation(s) at the position(s) disclosed herein, differs at no more than 10 positions with the amino acid sequence of SEQ ID NO: 1.
90. The EPO molecule of any of embodiments 1-89, which, other than the mutation(s) at the position(s) disclosed herein, differs at no more than 5 positions with the amino acid sequence of SEQ ID NO: 1.
91. The EPO molecule of any of embodiments 1-90, which, other than the mutation(s) at the position(s) disclosed herein, differs at no more than 4 positions with the amino acid sequence of SEQ ID NO: 1.
92. The EPO molecule of any of embodiments 1-91, which, other than the mutation(s) at the position(s) disclosed herein, differs at no more than 3 positions with the amino acid sequence of SEQ ID NO: 1.
93. The EPO molecule of any of embodiments 1-92, which, other than the mutation(s) at the position(s) disclosed herein, differs at no more than 2 positions with the amino acid sequence of SEQ ID NO: 1.
94. The EPO molecule of any of embodiments 1-93, which, other than the mutation(s) at the position(s) disclosed herein, differs at no more than 1 position with the amino acid sequence of SEQ ID NO: 1.
95. The EPO molecule of any of embodiments 1-94, which differs at no more than 15 positions with the amino acid sequence of SEQ ID NO: 1.
96. The EPO molecule of any of embodiments 1-95, which differs at no more than 10 positions with the amino acid sequence of SEQ ID NO: 1.
97. The EPO molecule of any of embodiments 1-96, which differs at no more than 5 positions with the amino acid sequence of SEQ ID NO: 1.
98. The EPO molecule of any of embodiments 1-97, which differs at no more than 4 positions with the amino acid sequence of SEQ ID NO: 1.
99. The EPO molecule of any of embodiments 1-98, which differs at no more than 3 positions with the amino acid sequence of SEQ ID NO: 1.
100. The EPO molecule of any of embodiments 1-99, which differs at no more than 2 positions with the amino acid sequence of SEQ ID NO: 1.
101. The EPO molecule of any of embodiments 1-100, which differs at no more than 1 position with the amino acid sequence of SEQ ID NO: 1.
102. The EPO molecule of any of embodiments 1-101, comprising the amino acid sequence of any of SEQ ID NOs: 3, 5, 7, 9, 11, 13, 15, 17, 19, 21, 23, 25, 27, 29, 31, 33, or 35, or an amino acid sequence that is at least 90%, 91%, 92%, 93%, 94%, 95%, 96%, 97%, 98%, or 99% identical thereto, or differs by no more than 1, 2, 3, 4, 5, 6, 7. 8, 9, or 10 amino acids therefrom.
103. The EPO molecule of any of embodiments 1-102, comprising the amino acid sequence of any of SEQ ID NOs: 3, 5, 7, 9, 11, 13, 15, 17, 19, 21, 23, 25, 27, 29, 31, 33, or 35.
104. The EPO molecule of any of embodiments 1-103, comprising or consisting of the amino acid sequence of SEQ ID NO: 3.
105. The EPO molecule of any of embodiments 1-103, comprising or consisting of the amino acid sequence of SEQ ID NO: 5.
106. The EPO molecule of any of embodiments 1-103, comprising or consisting of the amino acid sequence of SEQ ID NO: 7.
107. The EPO molecule of any of embodiments 1-103, comprising or consisting of the amino acid sequence of SEQ ID NO: 9.
108. The EPO molecule of any of embodiments 1-103, comprising or consisting of the amino acid sequence of SEQ ID NO: 11.
109. The EPO molecule of any of embodiments 1-103, comprising or consisting of the amino acid sequence of SEQ ID NO: 13.
110. The EPO molecule of any of embodiments 1-103, comprising or consisting of the amino acid sequence of SEQ ID NO: 15.
111. The EPO molecule of any of embodiments 1-103, comprising or consisting of the amino acid sequence of SEQ ID NO: 17.
112. The EPO molecule of any of embodiments 1-103, comprising or consisting of the amino acid sequence of SEQ ID NO: 19.
113. The EPO molecule of any of embodiments 1-103, comprising or consisting of the amino acid sequence of SEQ ID NO: 21.
114. The EPO molecule of any of embodiments 1-103, comprising or consisting of the amino acid sequence of SEQ ID NO: 23.
115. The EPO molecule of any of embodiments 1-103, comprising or consisting of the amino acid sequence of SEQ ID NO: 25.
116. The EPO molecule of any of embodiments 1-103, comprising or consisting of the amino acid sequence of SEQ ID NO: 27.
117. The EPO molecule of any of embodiments 1-103, comprising or consisting of the amino acid sequence of SEQ ID NO: 29.
118. The EPO molecule of any of embodiments 1-103, comprising or consisting of the amino acid sequence of SEQ ID NO: 31.
119. The EPO molecule of any of embodiments 1-103, comprising or consisting of the amino acid sequence of SEQ ID NO: 33.
120. The EPO molecule of any of embodiments 1-103, comprising or consisting of the amino acid sequence of SEQ ID NO: 35.
121. The EPO molecule of any of embodiments 1-120, having a reduced immunogenicity in a subject, as compared to a reference EPO molecule, e.g., an EPO molecule comprising the amino acid sequence of SEQ ID NO: 1, e.g., as determined in vitro, ex vivo, or in vivo.
122. The EPO molecule of embodiment 121, wherein the immunogenicity is reduced by at least 10%, 20%, 30%, 40%, 50%, 60%, 70%, 80%, or 90%, as compared to the reference EPO molecule.
123. The EPO molecule of embodiment 121 or 122, wherein the subject carries the HLA-DRB1*09 allele.
124. The EPO molecule of any of embodiments 1-123, which has the same, or substantially the same, biological activity, as compared to a reference EPO molecule, e.g., an EPO molecule comprising the amino acid sequence of SEQ ID NO: 1, e.g., as determined in vitro, ex vivo, or in vivo.

125. The EPO molecule of any of embodiments 1-124, which has a biological activity that is reduced by no more than 25%, 20%, 15%, 10%, 5%, or 2%, as compared to a reference EPO molecule, e.g., an EPO molecule comprising the amino acid sequence of SEQ ID NO: 1, e.g., as determined in vitro, ex vivo, or in vivo.

126. The EPO molecule of any of embodiments 124 or 125, wherein the biological activity is determined in an in vitro TF-1 proliferation assay.
127. The EPO molecule of any of embodiments 1-126, which is capable of having more glycoforms, e.g., at least 1, 2, 3, 4, or 5 more glycoforms, as compared to a reference EPO molecule, e.g., an EPO molecule comprising the amino acid sequence of SEQ ID NO: 1.
128. The EPO molecule of any of embodiments 1-127, which is capable of having 6, 7, 8, 9, 10, or more glycoforms.
129. The EPO molecule of any of embodiments 1-128, which is capable of having 7, 8, or more glycoforms.
130. The EPO molecule of any of embodiments 1-129, which has the same, or substantially the same, serum half-life, as compared to a reference EPO molecule, e.g., an EPO molecule comprising the amino acid sequence of SEQ ID NO: 1.
131. The EPO molecule of any of embodiments 1-130, which has an increased serum half-life, e.g., increased by at least 10%, 20%, 30%, 40%, 50%, 60%, 70%, 80%, or 90%, or at least 1, 2, 3, 4, 5, 6, 7, 8, 9, or 10-fold, as compared to a reference EPO molecule, e.g., an EPO molecule comprising the amino acid sequence of SEQ ID NO: 1.
132. The EPO molecule of any of embodiments 1-131, which has a reduced serum half-life, e.g., reduced by no more than 10%, 20%, 30%, 40%, 50%, 60%, 70%, 80%, or 90%, as compared to a reference EPO molecule, e.g., an EPO molecule comprising the amino acid sequence of SEQ ID NO: 1.
133. The EPO molecule of any of embodiments 1-132, which comprises a moiety that extends the serum half-life of an EPO polypeptide.

134. The EPO molecule of any of embodiments 1-133, which is a fusion protein.

135. The EPO molecule of any of embodiments 1-134, which comprises an Fc domain of an IgG.
136. The EPO molecule of embodiment 135, wherein the Fc domain covalently fused to an EPO polypeptide.
137. The EPO molecule of embodiment 136, wherein the N-terminus of the Fc domain is covalently fused to the C-terminus of the EPO polypeptide directly or indirectly.
138. A pharmaceutical composition comprising the EPO molecule of any of embodiments 1-137 and a pharmaceutically acceptable carrier.
139. A preparation comprising the EPO molecule of any of embodiments 1-137.
140. The preparation of embodiment 139, which is suitable for therapeutic use.
141. A kit comprising the EPO molecule of any of embodiments 1-137 and instructions for use. 142. A nucleic acid encoding the EPO molecule of any of embodiments 1-137.
143. A nucleic acid comprising the nucleotide sequence of any of SEQ ID NOs: 4, 6, 8, 10, 12, 14, 16, 18, 20, 22, 24, 26, 28, 30, 32, 34, or 36, or a nucleotide sequence that differs by no more than 5, 10, 15, 20, 25, 30, 35, 40, 45, or 50 nucleotides therefrom, or is at least 80%, 85%, 90%, 95%, 98%, or 99% identical thereto.
144. The nucleic acid of embodiment 143, comprising the nucleotide sequence of any of SEQ ID NOs: 4, 6, 8, 10, 12, 14, 16, 18, 20, 22, 24, 26, 28, 30, 32, 34, or 36.
145. A vector comprising the nucleic acid of any of embodiments 142-144.
146. The vector of embodiment 145, comprising a promoter operably linked to the nucleic acid.
147. A cell comprising the nucleic acid of any of embodiments 142-144.
148. A method of producing an EPO molecule, comprising culturing the cell of embodiment 147 under conditions that allows for expression of EPO polypeptide, thereby producing the EPO molecule.
149. A method of treating a disorder in a subject, comprising administering to the subject an effective amount of the EPO molecule of any of embodiments 1-137, thereby treating the disorder.
150. The method of embodiment 149, wherein the disorder is a disorder associated with EPO, e.g., a deficiency or abnormality of EPO.
151. The method of embodiment 150, wherein the disorder is anemia.
152. The method of embodiment 151, wherein the anemia is associated with or due to a chronic kidney disease (e.g., chronic renal failure), a zidovudine treatment, a chemotherapy, or a surgery.
153. A method of reducing an immunogenic response in a subject, comprising administering to a subject in need thereof an effective amount of the EPO molecule of any of embodiments 1-137, thereby reducing the immunogenic response.
154. A method of treating or preventing pure red-cell aplasia (PRCA) in a subject, comprising administering to a subject in need thereof an effective amount of the EPO molecule of any of embodiments 1-137, thereby treating or preventing PRCA.
155. A method of reducing an allogenic red blood cell (RBC) transfusion in a subject, comprising administering to a subject in need thereof an effective amount of the EPO molecule of any of embodiments 1-137, thereby reducing the allogenic RBC transfusion.
156. The method of any of embodiments 148-155, further comprising acquiring genotype information or a sequence that identifies an HLA genotype, e.g., the HLA-DRB1*09 genotype, in the subject, wherein the presence of the HLA genotype identifies the subject has, or is at risk of having, an increased immunogenicity to the EPO therapy.
157. The method of embodiment 156, wherein the EPO molecule is administered responsive to the acquisition of the genotype information.
158. A method of evaluating a subject, comprising acquiring genotype information that identifies an HLA genotype, e.g., the HLA-DRB1*09 genotype, in the subject, wherein the presence of the HLA genotype identifies the subject has, or is at risk of having, an increased immunogenicity to a therapy comprising an EPO molecule.
159. The method of embodiment 158, further comprising classifying the subject as a candidate for treatment with an EPO molecule disclosed herein.
160. The method of embodiment, 158 or 159, further comprising administering to the subject an effective amount of an EPO molecule disclosed herein.
161. The method of any of embodiments 148-160, wherein the subject carries, or has been determined to carry, the HLA-DRB1*09 allele.
162. The method of any of embodiments 148-161, wherein the subject has a chronic kidney disease, an HIV infection, or a cancer.
163. The method of any of embodiments 148-162, wherein the subject receives, or has received, zidovudine.
164. The method of any of embodiments 148-163, wherein the subject receives, or has received, a chemotherapy (e.g., a myelosuppressive chemotherapy), a surgery, or an allogenic RBC transfusion.
165. The method of any of embodiments 148-164, wherein the subject has, or has been determined to have, anti-EPO antibodies.
166. The method of any of embodiments 148-165, wherein the subject has been administered a different recombinant human EPO (rHuEPO), e.g., an EPO comprising the amino acid sequence of SEQ ID NO: 1.
167. The method of embodiment 166, wherein the administration of the different rHuEPO is discontinued or terminated.

### BRIEF DESCRIPTION OF THE DRAWINGS

FIG. 1A is an illustration of the amino acid sequence of EPO, indicating exemplary sites predicted to be contacted by the EPO receptor (EPO-R) (boldface); for binding to the HLA-DRB1*09-DQB1*03:09 allele (shaded); recognized by neutralizing antibodies (underlined); and considered for amino acid modification (marked with an asterisk above). FIG. 1A discloses SEQ ID NO: 46. FIG. 1B illustrates the structure of the EPO-R binding interface indicating a network of interacting hydrophobic amino acids including exemplary allele binding sites.

FIGS. 2A-2B are images of sodium dodecyl sulfate-polyacrylamide gel electrophoresis (SDS-PAGE) separations of exemplary EPO polypeptides, after Coomassie blue staining (FIG. 2A) and Western immunoblotting (FIG. 2B).

FIG. 3 is an image of an isoelectric focusing (IEF) gel separation of exemplary EPO polypeptides, indicating the number of isoforms in each.

FIG. 4 is a log dose-response curve indicating the in vitro bioactivity of exemplary EPO polypeptides in TF-1 cells.

FIGS. 5A-5C are bar graphs indicating the ex vivo immunogenicity of exemplary EPO polypeptides, as determined by the measurement of interferon gamma (IFN-γ) release using ELISA, providing the percentage of relative response (FIG. 5A), where asterisks denote a significant difference by t-test (*p≤0.05, **p≤0.01, ***p≤0.001, ****p≤0.0001); the IFN-γ levels for HLA-DRB1*09-positive volunteers (FIG. 5B); and the IFN-γ levels for HLA-DRB1*09-negative volunteers (FIG. 5C). Bar graphs are shown as mean±standard error.

### DETAILED DESCRIPTION

Recombinant human erythropoietin (rHuEPO) can be used as a biopharmaceutical drug given to subjects who have low levels of hemoglobin, for example, subjects with chronic kidney disease, cancer, HIV infection, or anemia. However, some subjects repeatedly receiving rHuEPO can develop an immune response to rHuEPO and the formation of anti-rHuEPO neutralizing antibodies, which can lead to the development of pure red cell aplasia (PRCA). Without wishing to be bound by theory, it is believed that in an embodiment, the immunogenic response to EPO is triggered by T-cells recognizing EPO epitopes bound to major histocompatibility complex (MHC) molecules displayed on the cell surface of antigen presenting cells (APCs). It is further believed that in an embodiment, there is an association between the development of anti-rHuEPO-associated PRCA and the HLA-DRB1*09 gene, which is entrenched in some populations, e.g., the Thai population. For example, screening of HLA-DRB1*09 in volunteers living in Bangkok, Thailand found that 22% volunteers have HLA-DRB1*09, confirming the high frequency of this particular HLA in Thailand.

The present disclosure is based, at least in part, on the discovery that modifying the amino acid sequence of the EPO polypeptide can reduce the immunogenicity of the EPO polypeptide, without disrupting its structural integrity or bioactivity. Disclosed herein are modified EPO polypeptides or EPO variants with reduced immunogenicity. Advantageously, the EPO polypeptides described herein can mimic one or more biological functions of natural EPO, e.g., to supplement or replace endogenous EPO in subjects, and/or treat or prevent an anemia or an EPO-associated disorder (e.g., an EPO deficiency-associated disorder) in a subject, without resulting in an immune reaction that may occur with other forms of recombinant human erythropoietin (rHuEPO). Without wishing to be bound by theory, it is believed that in an embodiment, the EPO polypeptides described herein can be used in subjects with a high risk of immunogenicity to rHuEPO, e.g., subjects who are dependent on rHuEPO and/or subjects who express particular class II MHC alleles, such as HLA-DRB1*09. For example, the EPO polypeptides described herein can be used to treat patients in populations that are at higher risk of immunogenicity to rHuEPO, e.g., the Thai population.

Also described herein are methods of screening EPO polypeptides (e.g., computationally and/or experimentally) for identification and characterization of immunogenic hotspots in the EPO polypeptide recognized by MHC alleles (e.g., HLA-DRB1*09). These methods can be further used for predicting mutants, for example having one or more (e.g., two, three, four, or more) mutations that reduce affinity for the allele. Additionally, nucleic acid molecules encoding the EPO polypeptides, expression vectors, host cells, compositions (e.g., pharmaceutical compositions), kits, containers, devices, and methods for making the EPO polypeptides, are also provided in the present disclosure. The EPO polypeptides and pharmaceutical compositions disclosed herein can be used, either alone or in combination with other agents or therapeutic modalities, to treat and/or prevent disorders and conditions, e.g., anemia or conditions associated with EPO deficiency, e.g., pure red cell aplasia (PRCA).

### Definitions

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% (e.g., within 4%, 3%, 2%, or 1%) of a given value or range of values.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Acquiring a sequence” as the term is used herein, refers to obtaining possession of a nucleotide sequence or amino acid sequence, by “directly acquiring” or “indirectly acquiring” the sequence. “Directly acquiring a sequence” means performing a process (e.g., performing a synthetic or analytical method) to obtain the sequence, such as performing a sequencing method (e.g., a Next Generation Sequencing (NGS) method). “Indirectly acquiring a sequence” refers to receiving information or knowledge of, or receiving, the sequence from another party or source (e.g., a third-party laboratory that directly acquired the sequence). The sequence acquired need not be a full sequence, e.g., sequencing of at least one nucleotide, or obtaining information or knowledge, that identifies a fusion molecule disclosed herein as being present in a subject constitutes acquiring a sequence.

Directly acquiring a sequence includes performing a process that includes a physical change in a physical substance, e.g., a starting material, such as a tissue sample, e.g., a biopsy, or a nucleic acid (e.g., DNA or RNA) sample. Exemplary changes include making a physical entity from two or more starting materials, shearing or fragmenting a substance, such as a genomic DNA fragment; separating or purifying a substance (e.g., isolating a nucleic acid sample from a tissue); combining two or more separate entities into a mixture, performing a chemical reaction that includes breaking or forming a covalent or non-covalent bond.

The compositions and methods disclosed herein encompass polypeptides and nucleic acids having the sequences specified, or sequences substantially identical or similar thereto, e.g., sequences at least 80%, 85%, 90%, 91%, 92%, 93%, 94%, 95%, 96%, 97%, 98%, 99%, 99.5%, 99.9% identical or higher to the sequence specified.

In the context of an amino acid sequence, the term “substantially identical” is used herein to refer to a first amino acid that contains a sufficient or minimum number of amino acid residues that are a) identical to, or b) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98%, 99%, 99.5%, or 99.9% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98%, 99%, 99.5%, or 99.9% identity to a reference sequence, e.g., a sequence provided herein.

The term “functional variant” refers polypeptides that have a substantially identical amino acid sequence to the naturally occurring sequence, or are encoded by a substantially identical nucleotide sequence, and can have one or more activities of the naturally occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a typical embodiment, the length of a reference sequence aligned for comparison purposes is at least 30%, e.g., at least 40%, 50%, 60%, 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In an embodiment, the percent identity between two amino acid or nucleotide sequences is determined using Clustal Omega (Sievers et al. Mol Syst Biol. 2011; 7:539). In an embodiment, the percent identity between two amino acid or nucleotide sequences is determined using Kalign2 (Lassmann et al. Nucleic Acids Res. 2009; 37(3):858-65; Lassmann and Sonnhammer BMC Bioinformatics. 2005; 6:298). In an embodiment, the percent identity between two amino acid or nucleotide sequences is determined using MAFFT (Katoh and Standley Mol Biol Evol. 2013; 30(4):772-80). In an embodiment, the percent identity between two amino acid or nucleotide sequences is determined using MUSCLE (Edgar Nucleic Acids Res. 2004; 32(5):1792-7; Edgar BMC Bioinformatics. 2004; 5:113). In an embodiment, the percent identity between two amino acid or nucleotide sequences is determined using MView (Brown et al. Bioinformatics. 1998; 14(4): 380-1). Other methods for determining the percent identity between two sequences are also described, e.g., in Li et al. Nucleic Acids Res. 2015; 43(W1):W580-4; McWilliam et al. Nucleic Acids Res. 2013; 41(Web Server issue):W597-600.

In an embodiment, the percent identity between two amino acid sequences is determined using the Needleman and Wunsch (J Mol Biol. 1970; 48(3):443-53) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In an embodiment, the percent identity between two nucleotide sequences is determined using the GAP program in the GCG software package (available at www.gcg.com), using an NWSgapdna. CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller (Comput. Appl. Biosci. 1988; 4(1):11-7) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases, for example, to identify other family members or related sequences. Such searches can be performed using the NBLAST and XBLAST programs (version 2.0) of Altschul, et al. 1990; J. Mol. Biol. 215:403-10. BLAST nucleotide searches can be performed with the NBLAST program, score=100, wordlength=12 to obtain nucleotide sequences homologous to a nucleic acid as described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., Nucleic Acids Res. 1997; 25:3389-3402. When utilizing BLAST and gapped BLAST programs, the default parameters of the respective programs (e.g., XBLAST and NBLAST) can be used. See www.ncbi.nlm.nih.gov.

It is understood that the molecules described herein may have additional conservative or non-essential amino acid substitutions, which do not have a substantial effect on their functions.

The term “allele” as used herein refers to an alternative form of a gene. For example, an allele described herein may be a naturally occurring sequence variant of a human gene. In an embodiment, the human gene is a human leukocyte antigen (HLA) gene, such as an HLA-A, —B, -DR, or -DQ allele, e.g., DRA, DRB1, DRB3/4/5, DQA-I, DQB1, DPA-I, or DPB1 allele, e.g., HLA-DRB1*09.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

The term “antigen presenting cell” or “APC” refers to an immune system cell such as an accessory cell (e.g., a B-cell, a dendritic cell, and the like) that displays an antigen complexed with a major histocompatibility complex (MHC), e.g., an MHC-II-epitope complex, on its surface. T-cells may recognize these complexes using their T-cell receptors (TCRs). APCs internalize and process antigens (e.g., into peptides (epitopes)) and present them to T-cells.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “immunogenicity” as used herein refers to the ability of a molecule (e.g., an antigen, epitope, or protein, e.g., an EPO polypeptide) to elicit an immune response (e.g., a humoral and/or cellular immune response) in a subject. An immune response may involve, inter alia, the production of antibodies (e.g., neutralizing or non-neutralizing antibodies), the formation of immune complexes, complement activation, mast cell activation, inflammation, and/or anaphylaxis. A skilled artisan can readily measure or determine the immunogenicity of a substance. For example, humoral-mediated immunological responses can be determined by measuring the level of antibodies in a biological sample (e.g., blood) using, inter alia, Western Blot, ELISA, or similar techniques. Cell-mediated immunological response can be determined, e.g., using proliferation assays (CD4⁺ T cells), cytotoxic T-lymphocyte assays, or immunohistochemistry methods.

The term “reduced immunogenicity” as used herein refers to a decreased ability to elicit an immune response, relative to the immunogenicity of a reference molecule (e.g., a wild type or naturally occurring EPO polypeptide, a BRP for EPO, or a modified EPO polypeptide that lacks one or more (e.g., all) of the mutations described herein). For example, a molecule (e.g., a protein, e.g., an EPO polypeptide) can have reduced immunogenicity if it elicits fewer neutralizing or non-neutralizing antibodies, e.g., as determined by antibody titer; exhibits decreased binding to one or more MHC alleles; or induces less T-cell activation or induces T-cell activation in a decreased number of subjects; as compared to a reference molecule (e.g., wild-type EPO).

The term “MHC-binding epitope” as used herein refer to an epitope (e.g., a peptide) that is capable of binding to one or more class II MHC alleles with sufficient affinity to enable the formation of an MHC-epitope-T-cell receptor complex and initiate T-cell activation. An MHC-binding epitope described herein can be a peptide sequence (e.g., a linear peptide sequence) comprising at least about 9 amino acid residues, e.g., at least about 9, 10, 11, 12, 13, 14, 15, 16, 17, or 18, amino acid residues. In an embodiment, an MHC-binding epitope is a peptide sequence comprising about 9 amino acid residues.

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably herein. These terms refer to a polymeric form of nucleotides of any length, either deoxyribonucleotides or ribonucleotides, or analogs thereof. The polynucleotide may be either single-stranded or double-stranded, and if single-stranded, may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, a “subject” refers to a human or animal. The animal is typically a vertebrate such as a primate, rodent, domestic animal, or game animal. Primates include chimpanzees, cynomolgus monkeys, spider monkeys, and macaques (e.g., Rhesus). Rodents include mice, rats, woodchucks, ferrets, rabbits and hamsters. Domestic and game animals include cows, horses, pigs, deer, bison, buffalo, feline species (e.g., domestic cat), canine species (e.g., dog, fox, wolf), avian species (e.g., chicken, emu, ostrich), and fish (e.g., trout, catfish and salmon). In certain embodiments of the present disclosure, the subject is a mammal (e.g., a primate, e.g., a human). A subject can be male or female. In certain embodiments, the subject is a mammal. The mammal can be a human, non-human primate, mouse, rat, dog, cat, horse, or cow, but are not limited to these examples. In an embodiment, the term “subject” is intended to include living organisms in which an immune response can be elicited (e.g., a mammal, e.g., human subject).

As used herein, the term “treat,” e.g., an anemia or an EPO associated disorder (e.g., an EPO deficiency-associated disorder) (e.g., pure red cell aplasia), means that a subject (e.g., a human) who has a disorder, e.g., an anemia or an EPO associated disorder (e.g., an EPO deficiency-associated disorder) and/or experiences a symptom of a disorder, e.g., an anemia or an EPO associated (e.g., an EPO deficiency-associated) disorder, will, in an embodiment, suffer a less severe symptom and/or recover faster when an EPO polypeptide (e.g., an EPO polypeptide described herein) is administered than if the EPO polypeptide was never administered. In an embodiment, when an anemia or an EPO associated disorder (e.g., an EPO deficiency-associated disorder), is treated, the level or activity of hemoglobin may be increased in a treated subject compared to a comparable untreated subject. Methods for determining hemoglobin levels have been described (see, e.g., Billett H. H. (1990) Hemoglobin and Hematocrit. (Walker H K, Hall W D, Hurst J W, editors). Clinical Methods: The History, Physical, and Laboratory Examinations. 3rd edition, Chapter 151. Boston: Butterworths). Treatment can, e.g., partially or completely, alleviate, ameliorate, relieve, inhibit, or reduce the severity of, and/or reduce incidence, and optionally, delay onset of, one or more manifestations of the effects or symptoms, features, and/or causes of a disorder, e.g., an anemia or an EPO associated disorder (e.g., an EPO deficiency-associated disorder).

In an embodiment, treatment can partially or completely, alleviate, ameliorate, relieve, inhibit, or reduce the severity of, and/or reduce incidence, and optionally, delay onset of, immunogenicity to an EPO polypeptide (e.g., recombinant human EPO or endogenous human EPO), in a subject. In an embodiment, when anemia or an EPO associated disorder (e.g., an EPO deficiency-associated disorder), is treated with an EPO polypeptide (e.g., an EPO polypeptide described herein), the immune response in the subject may be reduced or eliminated, compared to the immune response in a comparable subject treated with a different EPO polypeptide (e.g., rHuEPO, e.g., epoetin alfa, beta, delta, or omega). For example, a diagnostic method using enzyme-linked immunosorbent assay (ELISA) can detect the level of T-cell response attributed to different EPO polypeptides (e.g., by quantitating levels of interferon gamma (IFN-γ) release), as a measure of the immunogenicity of the EPO polypeptide. Other assays, e.g., antibody titers, can be used to monitor treatment in a patient, or detect the presence of an immune response.

In an embodiment, treatment is of a subject who does not exhibit certain signs of a disorder, e.g., an anemia or an EPO associated disorder (e.g., an EPO deficiency-associated disorder), and/or of a subject who exhibits only early signs of a disorder, e.g., an anemia or an EPO associated disorder (e.g., an EPO deficiency-associated disorder). In an embodiment, treatment is of a subject who exhibits one or more established signs of a disorder, e.g., an anemia or an EPO associated disorder (e.g., an EPO deficiency-associated disorder). In an embodiment, treatment is of a subject diagnosed as suffering from a disorder, e.g., an anemia or an EPO associated disorder (e.g., an EPO deficiency-associated disorder). In an embodiment, the disorder is an EPO associated disorder (e.g., an EPO deficiency-associated disorder) described herein. In an embodiment, the disorder is pure red cell aplasia.

As used herein, the term “prevent,” a disorder, e.g., an anemia or an EPO associated disorder (e.g., an EPO deficiency-associated disorder) (e.g., pure red cell aplasia), means that a subject (e.g., a human) is less likely to have the disorder, e.g., an anemia or an EPO-associated disorder (e.g., an EPO deficiency-associated disorder), if the subject receives the EPO polypeptide (e.g., an EPO polypeptide described herein). In an embodiment, the subject is at risk of developing the disorder, e.g., an anemia or an EPO-associated disorder (e.g., an EPO deficiency-associated disorder). In an embodiment, the subject is at risk of developing pure red cell aplasia. In an embodiment, the disorder is an EPO-associated disorder (e.g., an EPO deficiency-associated disorder) described herein. In an embodiment, the disorder is pure red cell aplasia.

The terms “variant erythropoietin (EPO) polypeptide,” “modified EPO polypeptide,” “EPO variant,” “EPO mutant,” and “EPO mutein” are used herein interchangeably, and refer to non-naturally occurring polypeptides that differ from a reference EPO polypeptide, e.g., a wild-type or parent EPO polypeptide by at least one residue (e.g., differ by at least one amino acid insertion, deletion, or substitution). EPO variants are characterized by the predetermined nature of the variation, that differentiates the protein from a naturally occurring allelic or interspecies variation of the EPO polypeptide sequence. An EPO variant described herein may exhibit one or more biological properties (e.g., one or more biological activities) that is comparable to naturally occurring EPO. Further, an EPO variant described herein may have been engineered to possess one or more different biological properties (e.g., reduced immunogenicity) as compared to a reference EPO polypeptide, e.g., a wild type or naturally occurring EPO. In an embodiment, EPO variants contain an insertion, a deletion, and/or a substitution, which may be at any position in the amino acid sequence, e.g., at the N-terminus, C-terminus, or internally. In an embodiment, an EPO variant comprises at least 1 residue that is different with respect to a reference EPO polypeptide, e.g., a wild type or naturally occurring EPO polypeptide, e.g., a difference of at least 1, 2, 3, 4, 5, or more amino acid residues. In an embodiment, an EPO variant comprises 1, 2, 3, or 4 amino acid residues that are different with respect to a reference EPO polypeptide, e.g., a wild type or naturally occurring EPO polypeptide. In an embodiment, an EPO variant is subjected to co- or post-translational modification, e.g., synthetic derivatization of one or more amino-acid side chains or termini, glycosylation, PEGylation, circular permutation, cyclization, fusion to proteins or protein domains (e.g., immunoglobulin), or addition of a peptide tag or label.

The term “erythropoietin molecule” or “EPO molecule” as used herein can refer to a molecule that comprise a full-length EPO polypeptide or a functional fragment thereof.

As used herein, the terms “variant erythropoietin (EPO) nucleic acid,” “modified EPO nucleic acid,” and “variant EPO nucleic acid” are used herein interchangeably, and refer to nucleic acids encoding an erythropoietin protein variant (or a modified EPO polypeptide). Due to degeneracy in the genetic code, a large number of nucleic acids may encode the variant erythropoietin proteins of the present disclosure, e.g., by modifying the sequence of one or more codons in a way that does not change the amino acid sequence of the corresponding variant erythropoietin protein.

The term “wild type,” “wild-type,” or “WT” as used herein refers to a polypeptide, protein, or nucleic acid containing a naturally occurring amino acid sequence or a nucleotide sequence, including allelic variations, such as an amino acid sequence or nucleotide sequence that has not been modified or engineered. For example, a wild type EPO polypeptide may refer to any EPO polypeptide comprising a naturally occurring EPO amino acid sequence, including an isolated EPO polypeptide, a recombinant EPO polypeptide, or a synthetic EPO polypeptide, that comprises a naturally occurring amino acid sequence.

Ranges: throughout this disclosure, various embodiments can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3.7, 3, 4, 5, 5.6, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

Various embodiments of the compositions and methods herein are described in further detail below. Additional definitions are set out throughout the specification.

### Erythropoietin (EPO)

### Human Erythropoietin (EPO)

Erythropoietin (EPO) is a glycoprotein hormone produced in the kidneys, that plays an essential role in the production and maturation of red blood cells (RBCs). The mature amino acid sequence of human EPO typically contains 165 amino acid residues and has a relative molecular mass of about 30,600 Daltons. The EPO structure has a 4-helical bundle topology, comprising helices A, B, C and D. Glycosylation of EPO occurs during post-translational modification, and involves the addition of three N-linked carbohydrate chains and one O-linked carbohydrate chain, and an increase in the original molecular mass of approximately 40%. The glycosylation of EPO is known to affect stability, solubility, and bioactivity of the protein.

The amino acid and nucleotide sequences of human EPO are known in the art. An exemplary amino acid sequence of wild type human EPO is provided as follows.

(SEQ ID NO: 1)
APPRLICDSRVLERYLLEAKEAENITTGCAEHCSLNENITVPDTKVNFY
AWKRMEVGQQAVEVWQGLALLSEAVLRGQALLVNSSQPWEPLQLHVDKA
VSGLRSLITLLRALGAQKEAISPPDAASAAPLRTITADTFRKLFRVYSN
FLRGKLKLYTGEACRTGDR

The EPO amino acid position numbering used herein are generally based on the amino acid sequence of SEQ ID NO: 1.

Recombinant Human Erythropoietin (rHuEPO)

Recombinant human erythropoietin (rHuEPO or rhEPO), sometimes known as an erythropoiesis-stimulating agent (ESA), can be a biopharmaceutical comprising an engineered form of EPO, for example, having the same or altered amino acid sequence as human EPO. rHuEPO is commonly administered to subjects that have low hemoglobin levels (anemia), e.g., due to underlying disease or therapy. There are several disease areas and related uses for exogenous EPO polypeptides such as rHuEPO. For example, subjects with chronic kidney disease (CKD) are commonly administered rHuEPO to treat associated anemia. Other diseases commonly associated with rHuEPO treatment include HIV infections, AIDS, and cancer. Additionally, subjects who are dependent on dialysis, or are receiving chemotherapy, radiation, or AIDS or HIV therapy are commonly treated with rHuEPO. Additionally, rHuEPO can be administered subjects undergoing or having had surgery or transplantation (e.g., bone marrow transplantation), to promote erythropoiesis. rHuEPO can also be used as a performance-enhancing drug, and several forms of EPO are prohibited from in sports by the World Anti-Doping Agency. Non-limiting examples of rHuEPO include epoetin alfa (e.g., EPOGEN®, EPOADE®, EPOPEN®, EPDXITIN®, EPREX®, ERYPRO®, ESPO®, GLOBUREN®, PROCRIT®), epoetin beta (e.g., EPOCH, EPOGIN®, ERANTIN®, ERITROGEN®, MAROGEN®, NEORECORMON®, RECORMON®), epoetin delta (e.g., DYNEPO®), and epoetin omega (e.g., EPOMAX®, HEMAX®).

As described in more detail herein, rHuEPO can lead to an immune reaction in some individuals, particularly due to genetic factors, in addition to factors related to the pharmaceutical formulation (e.g., presence of aggregates) and/or the frequency of administration (e.g., in rHuEPO-dependent subjects). The immunogenicity of rHuEPO can have severe consequences, e.g., causing pure red cell aplasia (PRCA). Indeed, since the use of rHuEPO for treatment of anemia of patients having chronic kidney disease (CKD) has become more widespread, some CKD patients who have previously or are currently using rHuEPO have been reported to display suspected or confirmed PRCA. For example, a report in 2005 indicated that four Thai patients who received rHuEPO therapy had a confirmed anti-rHuEPO associated PRCA, and each subject also displayed the HLA-DRB1*09 allele (Praditpornsilpa et al., vide supra). As described herein, the DRB1*09 allele has a high frequency among Southeast Asian countries. Without wishing to be bound by theory, it is believed that in an embodiment, the disruption between EPO epitope and HLA-DRB1*09 molecule can lower immunogenicity in patients with HLA-DRB1*09.

### EPO Variants

Disclosed herein are variant EPO polypeptides that can be used, e.g., to treat or prevent anemias and/or disorders associated with EPO, e.g., EPO-deficiencies, or symptoms thereof. For example, the EPO polypeptides can have one or more structural and/or functional properties described herein. In an embodiment, the EPO polypeptides described herein comprise one or more mutations, e.g., one or more mutations described herein. In an embodiment, the EPO polypeptide has a lower immunogenicity, e.g., as compared to a reference EPO polypeptide or preparation, e.g., a wild-type or naturally occurring EPO polypeptide, a Biological Reference Preparation (BRP) for EPO, or a modified EPO polypeptide that lacks one or more (e.g., all) mutations described herein. BRP for EPO is described, e.g., in Ferguson et al. Pharmeur Bio Sci Notes. 2019; 2019:27-33; Burns et al. Pharmeur Bio Sci Notes. 2015; 2015:246-53; Behr-Gross et al. Pharmeuropa Bio. 2007 December; 2007(1):49-66; Behr-Gross et al. Pharmeuropa Bio. 2004 December; 2004(1):23-33.

In an embodiment, the EPO polypeptides described herein can be used as alternatives to conventional rHuEPO, e.g., to avoid or reduce immunogenicity typically associated with rHuEPO, and/or to supplement endogenous EPO, such as in subjects who are deficient in endogenous EPO. In an embodiment, the EPO polypeptide is administered to a subject having, or at risk of having, pure-red cell aplasia (e.g., rHuEPO-induced PRCA). In an embodiment, the EPO polypeptide is used to treat or prevent pure-red cell aplasia (e.g., rHuEPO-induced PRCA) in a subject, e.g., a human subject.

### Class II MHC Allele Binding Sites

In an embodiment, the EPO polypeptides described herein comprise an amino acid sequence tailored to reduce immunogenicity in a subject. For example, the EPO polypeptides described herein can be administered to a subject to lower (e.g., eliminate) the immune response in the subject, relative to the immune response elicited in a comparable subject administered with a reference rHuEPO. In an embodiment, the subject is susceptible to immunogenicity to rHuEPO, e.g., a subject having a genetic background described herein, e.g., possessing an HLA-DRB1*09 allele.

In an embodiment, the EPO polypeptide comprises a modified binding site, such that the EPO variant exhibits a lower affinity for a class II MHC allele (e.g., the HLA-DRB1*09 allele). The binding site may comprise a region of about 9 amino acid residues. For example, binding sites predicted by a method described herein can be at one or more of positions 74-82, 102-110, 108-116, 142-150, or 145-153 of the EPO amino acid sequence. In an embodiment, the binding site comprises an amino acid sequence provided in Table 1. In an embodiment, the EPO polypeptide comprises one or more (e.g., 2, 3, 4, 5, or more) mutations in one or more (e.g., 2, 3, 4, or all) of SEQ ID NOS: 37-41. In an embodiment, the EPO polypeptide comprises one or more (e.g., 2, 3, 4, 5, or more) mutations in SEQ ID NO: 37. In an embodiment, the EPO polypeptide comprises one or more (e.g., 2, 3, 4, 5, or more) mutations in SEQ ID NO: 38. In an embodiment, the EPO polypeptide comprises one or more (e.g., 2, 3, 4, 5, or more) mutations in SEQ ID NO: 39. In an embodiment, the EPO polypeptide comprises one or more (e.g., 2, 3, 4, 5, or more) mutations in SEQ ID NO: 40. In an embodiment, the EPO polypeptide comprises one or more (e.g., 2, 3, 4, 5, or more) mutations in SEQ ID NO: 41.

TABLE 1
Exemplary HLA-DRB1*09-DQB1*03:09
allele binding sites in EPO

| SEQ ID NO | Amino Acid Sequence | Amino Acid Position |
|---|---|---|
| 37 | VLRGQALLV | 74-82 |
| 38 | LRSLTTLLR | 102-110 |
| 39 | LLRALGAQK | 108-116 |
| 40 | FRVYSNFLR | 142-150 |
| 41 | YSNFLRGKL | 145-153 |

### EPO Mutations

Mutations can be introduced to an EPO polypeptide amino acid sequence to affect its biological properties, e.g., bioactivity and or immunogenicity. For example, a binding site of the EPO sequence (e.g., predicted by methods described herein) can be modified, e.g., by substitution, insertion, or deletion of an amino acid, e.g., to disrupt binding, or lower the affinity, between the EPO binding site and one or more class II MHC alleles.

In an embodiment, the EPO polypeptide comprises a mutation at one or more residues in the binding side. In an embodiment, the EPO polypeptide comprises a mutation at one or more amino acid positions selected from positions 12-152 of the EPO amino acid sequence. In an embodiment, the EPO polypeptide comprises a mutation at one or more amino acid positions selected from positions 74-82, 102-116, or 142-153 of the EPO amino acid sequence. In an embodiment, the EPO polypeptide comprises a mutation at one or more amino acid positions selected from positions 74-82. In an embodiment, the EPO polypeptide comprises a mutation at one or more amino acid positions selected from positions 102-110. In an embodiment, the EPO polypeptide comprises a mutation at one or more amino acid positions selected from positions 108-116. In an embodiment, the EPO polypeptide comprises a mutation at one or more amino acid positions selected from positions 142-150. In an embodiment, the EPO polypeptide comprises a mutation at one or more amino acid positions selected from positions 145-153.

In an embodiment, the EPO polypeptide comprises a mutation at one or more (e.g., 2, 3, 4, or more) amino acid positions selected from positions 74, 75, 76, 77, 78, 79, 80, 81, or 82 in the EPO amino acid sequence. In an embodiment, an EPO polypeptide comprises a mutation at one or more (e.g., 2, 3, 4, or more) amino acid positions selected from positions 102, 103, 104, 105, 106, 107, 108, 109, 110, 111, 112, 113, 114, 115, or 116 in the EPO amino acid sequence. In an embodiment, the EPO polypeptide comprises a mutation at one or more (e.g., 2, 3, 4, or more) amino acid positions selected from positions 142, 143, 144, 145, 146, 147, 148, 149, 150, 151, 152, or 153 in the EPO amino acid sequence. In an embodiment, the EPO polypeptide comprises a mutation at one or more (e.g., 2, 3, 4, or more) amino acid positions selected from positions 74, 75, 76, 77, 79, 80, 81, 82, 102, 105, 106, 109, 111, 112, 113, 114, 115, 116, 142, 145, 146, 148, 149, 152 and 153 in the EPO amino acid sequence. In an embodiment, the EPO polypeptide comprises a mutation at one or more (e.g., 2, 3, 4, or more) amino acid residues selected from positions 12, 15, 67, 70, 74, 102, 105, 106, 109, 114, 146, 149, or 152.

In an embodiment, the EPO variant comprises an amino acid sequence comprising a substitution at one or more positions of the human EPO amino acid sequence.

In an embodiment, the polypeptide comprises a substitution at position 12, e.g., an L12A or L12I substitution. In an embodiment, the polypeptide comprises a substitution at position 15, e.g., a Y15I or Y15W substitution. In an embodiment, the polypeptide comprises a substitution at position 67, e.g., an L67D, L67N, or L67V substitution. In an embodiment, the polypeptide comprises a substitution at position 70, e.g., an L70V substitution. In an embodiment, the polypeptide comprises a substitution at position 74, e.g., a V74D, V74L, or V74N substitution. In an embodiment, the polypeptide comprises a substitution at position 102, e.g., an L102D, L102I, L102N, L102T, or L102V substitution. In an embodiment, the polypeptide comprises a substitution at position 105, e.g., an L105F or L105S substitution. In an embodiment, the polypeptide comprises a substitution at position 106, e.g., a T106A, T106G, or T106H substitution. In an embodiment, the polypeptide comprises a substitution at position 109, e.g., an L109A, L109C, or L109W substitution. In an embodiment, the polypeptide comprises a substitution at position 114, e.g., an A114C substitution. In an embodiment, the polypeptide comprises a substitution at position 146, e.g., an S146N substitution. In an embodiment, the polypeptide comprises a substitution at position 149, e.g., an L149V or L149W substitution. In an embodiment, the polypeptide comprises a substitution at position 152, e.g., a K152Q substitution.

In an embodiment, the polypeptide comprises mutations (e.g., substitutions) at positions 67 and 102. In an embodiment, the polypeptide comprises mutations (e.g., substitutions) at positions 70, 74, and 102. In an embodiment, the polypeptide comprises mutation (e.g., substitution) at position 102. In an embodiment, the polypeptide comprises mutations (e.g., substitutions) at positions 67, 74, and 102. In an embodiment, the polypeptide comprises mutations (e.g., substitutions) at positions 12, 15, 105, and 149. In an embodiment, the polypeptide comprises mutation (e.g., substitution) at position 105. In an embodiment, the polypeptide comprises mutation (e.g., substitution) at position 106. In an embodiment, the polypeptide comprises mutation (e.g., substitution) at position 109. In an embodiment, the polypeptide comprises mutations (e.g., substitutions) at positions 109 and 114. In an embodiment, the polypeptide comprises mutations (e.g., substitutions) at positions 102, 105, 106, and 109. In an embodiment, the polypeptide comprises mutations (e.g., substitutions) at positions 146 and 152.

In an embodiment, the polypeptide comprises a substitution of leucine to valine, e.g., at position(s) 67, 70, 102, and/or 149. In an embodiment, the polypeptide comprises a substitution of valine to leucine, e.g., at position 74. In an embodiment, the polypeptide comprises a substitution of leucine to isoleucine, e.g., at position(s) 12 and/or 102. In an embodiment, the polypeptide comprises a substitution of leucine to threonine, e.g., at position 102. In an embodiment, the polypeptide comprises a substitution of leucine to asparagine, e.g., at position(s) 67 and/or 102. In an embodiment, the polypeptide comprises a substitution of leucine to aspartic acid, e.g., at position(s) 67 and/or 102. In an embodiment, the polypeptide comprises a substitution of leucine to alanine, e.g., at position(s) 12 and/or 109. In an embodiment, the polypeptide comprises a substitution of leucine to phenylalanine, e.g., at position 105. In an embodiment, the polypeptide comprises a substitution of leucine to serine, e.g., at position 105. In an embodiment, the polypeptide comprises a substitution of leucine to tryptophan, e.g., at position(s) 109 and/or 149. In an embodiment, the polypeptide comprises a substitution of leucine to cysteine, e.g., at position 109. In an embodiment, the polypeptide comprises a substitution of threonine to alanine, e.g., at position 106. In an embodiment, the polypeptide comprises a substitution of threonine to glycine, e.g., at position 106. In an embodiment, the polypeptide comprises a substitution of threonine to histidine, e.g., at position 106. In an embodiment, the polypeptide comprises a substitution of valine to asparagine, e.g., at position 74. In an embodiment, the polypeptide comprises a substitution of valine to aspartic acid, e.g., at position 74. In an embodiment, the polypeptide comprises a substitution of tyrosine to isoleucine, e.g., at position 15. In an embodiment, the polypeptide comprises a substitution of tyrosine to tryptophan, e.g., at position 15. In an embodiment, the polypeptide comprises a substitution of alanine to cysteine, e.g., at position 114. In an embodiment, the polypeptide comprises a substitution of serine to asparagine, e.g., at position 146. In an embodiment, the polypeptide comprises a substitution of lysine to glutamine, e.g., at position 152.

In an embodiment, the polypeptide comprises L67V and L102V substitutions. In an embodiment, the polypeptide comprises L70V, V74L, and L1021 substitutions. In an embodiment, the polypeptide comprises an L102T substitution. In an embodiment, the polypeptide comprises L67N, V74N, and L102D substitutions. In an embodiment, the polypeptide comprises L67D, V74D, and L102N substitutions. In an embodiment, the polypeptide comprises L12A, Y151, L105F, and L149W substitutions. In an embodiment, the polypeptide comprises L12L Y15W, L105S, and L149V substitutions. In an embodiment, the polypeptide comprises an L105F substitution. In an embodiment, the polypeptide comprises a T106A substitution. In an embodiment, the polypeptide comprises a T106G substitution. In an embodiment, the polypeptide comprises a T106H substitution. In an embodiment, the polypeptide comprises an L109A substitution. In an embodiment, the polypeptide comprises an L109W substitution. In an embodiment, the polypeptide comprises L109C and A114C substitutions. In an embodiment, the polypeptide comprises L102T, L105S, T106H, and L109W substitutions. In an embodiment, the polypeptide comprises L102T, L105S, T106G, and L109W substitutions. In an embodiment, the polypeptide comprises S146N and K152Q substitutions.

An EPO polypeptide (e.g., EPO variant) described herein may comprise a mutation on any one of the four helices. In an embodiment, the EPO polypeptide comprises a mutation within one or more of the A, B, C, and D helices. In an embodiment, the EPO polypeptide comprises a mutation within helix A. In an embodiment, the EPO polypeptide comprises a mutation within helix B. In an embodiment, the EPO polypeptide comprises a mutation within helix C. In an embodiment, the EPO polypeptide comprises a mutation within helix D. In an embodiment, the EPO polypeptide comprises a mutation within helices B and C. In an embodiment, the mutations possessed by an EPO variant, e.g., having in vitro bioactivity (e.g., EPO-1.2, EPO-3.1, EPO-3.2, EPO-3.3 or EPO-4.1 shown in Table 2) are located within helix B and/or helix C.

Without wishing to be bound by theory, in an embodiment, amino acid positions 74, 102, 106 and 109 are believed to be epitopes for HLA-DR-B 1. In an embodiment, substitution of leucine to valine at position 70, valine to leucine at position 74, leucine to isoleucine at position 102 (EPO-1.2) and leucine to alanine at position 109 (EPO-4.1) preserve the hydrophobic network of EPO. Any suitable substitution between non-polar amino acid groups can be incorporated in an EPO polypeptide, e.g., between alanine, leucine, valine and isoleucine.

In an embodiment, a single mutation (e.g., as in EPO-3.1, EPO-3.2 and EPO-3.3 of Table 2) alters the native amino acid character. For example, a polar hydroxyl-containing amino acid such as threonine, e.g., a threonine at position 106, can be substituted to smaller and/or non-polar amino acids, such as alanine (e.g., in EPO-3.1) or glycine (e.g., in EPO-3.2). Additionally, a heteroaromatic moiety (e.g., an imidazole) can be introduced, e.g., at position 106, to avoid or reduce recognition by an MHC II allele (e.g., in EPO-3.3). The EPO polypeptides described herein, e.g., containing these mutated sequences, may avoid or reduce recognition by an MHC II molecule, leading to the lower immunogenic response in a subject possessing the MHC II molecule that receives said EPO polypeptide.

In an embodiment, the mutation results in the EPO polypeptide having reduced HLA-binding affinity, relative to a reference EPO polypeptide or preparation (e.g., wild-type or naturally occurring EPO, a BRP for EPO, or a modified EPO polypeptide that lacks one or more (e.g., all) of the mutations described herein). In an embodiment, the mutation results in the EPO polypeptide having reduced binding affinity to the HLA-DRB1*09 allele. In an embodiment, the EPO polypeptide has an HLA-binding affinity (e.g., HLA-DRB1*09 allele binding affinity) of about 0.01%, 0.02%, 0.05%, 0.1%, 0.2%, 0.5%, 1%, 2%, 5%, 10%, 20%, 30%, 40%, 50%, 60%, 70%, 80%, relative to the HLA binding affinity of a reference EPO polypeptide or preparation (e.g., wild-type or naturally occurring EPO, a BRP for EPO, or a modified EPO polypeptide that lacks one or more (e.g., all) of the mutations described herein). In an embodiment, the EPO polypeptide has an HLA-binding affinity of less than about 90%, e.g., less than about 80%, 70%, 60%, 50%, 40%, 30%, 20%, 10%, 5%, 4%, 3%, 2%, 1%, 0.5% or less, relative to the HLA-binding affinity of a reference EPO polypeptide or preparation (e.g., wild-type or naturally occurring EPO, a BRP for EPO, or a modified EPO polypeptide that lacks one or more (e.g., all) of the mutations described herein).

### EPO Isoforms

The EPO preparations described herein can comprise one or more isoforms (e.g., glycoforms) of an EPO polypeptide described herein. For example, rHuEPO polypeptides generally possess complex glycosylation patterns and exists as several different isoforms, and fully glycosylated EPO is known to contain as many as 14 isoforms. In an embodiment, a biological reference preparation of EPO (Burns, C. et al. Pharmeur. Bio. Sci. Notes (2015) 246-253) comprises 6 different isoforms. In an embodiment, the EPO preparation comprises 6 or more different isoforms, e.g., between 6 to 8 isoforms. Without wishing to be bound by theory, it is believed that in an embodiment this variation of isoform (e.g., glycoform) profile can be due to the difference in protein sequence and/or posttranslational modifications of the glycan moieties, e.g., by the different passage of transfected cells.

In an embodiment, the EPO preparation comprises a single isoform. In an embodiment, an EPO polypeptide described herein comprises a mixture of isoforms, e.g., 2, 3, 4, 5, 6, 7, 8, 9, 10, 11, 12, 13, 14, or more isoforms. In an embodiment, the EPO preparation comprises a higher number of isoforms compared to an EPO preparation comprising unmodified EPO polypeptides (e.g., wild-type or naturally occurring EPO polypeptides). In an embodiment, the EPO preparation comprises six isoforms. In an embodiment, the EPO preparation comprises seven isoforms. In an embodiment, the EPO preparation comprises eight isoforms.

In an embodiment, the EPO preparation comprises a mixture of glycoforms, each glycoform possessing varied glycan moieties, e.g., variation in the degree of branching of glycans, glycan structure (e.g., different carbohydrates or glycosidic linkages), the number of N- and O-linked glycans, and/or the number of sialic acid moieties.

The isoform (e.g., glycoform) distribution of an EPO polypeptide can be determined using known techniques, e.g., by separation using an isoelectric focusing (IEF) gel. For example, an EPO preparation comprising a mixture of isoforms can be separated by IEF gel, and the number of different bands visualized on the gel can be used to determine the number of isoforms. Different isoforms can be separated, e.g., according to their relative acidity or basicity.

Without wishing to be bound by theory, it is believed that in an embodiment, the isoform distribution can be caused by the differences in the microheterogeneity of glycosylation between EPO polypeptides. Variation in the glycoforms of EPO polypeptides, e.g., the number of sialic acid molecules, can sometimes greatly affect the biological activity of the EPO polypeptide including receptor binding affinity and serum clearance. As such, desirable features can be obtained by selecting an EPO polypeptide having a particular glycosylation profile, e.g., to increase bioactivity and/or decrease serum clearance of the EPO polypeptide. Such advantages may provide the opportunity to administer the EPO polypeptide less frequently, relative to conventional therapy (e.g., using rHuEPO). In an embodiment, the EPO polypeptide has the same serum half-life as compared to an unmodified EPO polypeptide (e.g., wild-type or naturally occurring EPO), a BRP for EPO, or a modified EPO that lacks one or more (e.g., all) of the mutations described herein. In an embodiment, the EPO polypeptide has reduced serum half-life as compared to an unmodified EPO polypeptide (e.g., wild-type or naturally occurring EPO), a BRP for EPO, or a modified EPO that lacks one or more (e.g., all) of the mutations described herein. In an embodiment, the EPO polypeptide has increased serum half-life as compared to an unmodified EPO polypeptide (e.g., wild-type or naturally occurring EPO), a BRP for EPO, or a modified EPO that lacks one or more (e.g., all) of the mutations described herein.

### Additional EPO Polypeptide

In an embodiment, an EPO polypeptide described herein is modified, e.g., by post-translational modification. In an embodiment, the modification comprises glycosylation, deglycosylation, carboxylation, hydroxylation, carbamylation, sulfation, prenylation, farnesylation, or conjugation to another molecule, such as a polymer, polypeptide or protein (e.g., albumin, immunoglobulin, polyethylene glycol (PEG), or hydroxyalkyl starch). For example, an EPO polypeptide described herein can be conjugated (e.g., covalently attached) to a protein or protein domain. In an embodiment, the EPO polypeptide is conjugated to albumin. In an embodiment, the EPO polypeptide is conjugated to a histidine tag (e.g., a 6xHis tag (SEQ ID NO: 43)). In an embodiment, the EPO polypeptide is conjugated to one or more of glutathione S-transferase, hemagglutinin, a FLAG epitope, beta-galactosidase, luciferase, a green fluorescent protein, or the like.

In an embodiment, the EPO polypeptide is covalently attached to an antibody (e.g., an immunoglobulin (IgG) molecule). In an embodiment, the EPO polypeptide is covalently attached to an IgG molecule. In an embodiment, the EPO polypeptide is covalently attached to the Fc portion of an IgG molecule.

The EPO polypeptide can be linked to another molecule (e.g., a protein, polypeptide, or polymer) by any suitable chemical means. The EPO polypeptide can be linked to the other molecule directly (e.g., by a covalent bond), or by a linker. For example, linkage of the EPO polypeptide to another molecule (e.g., a protein) may involve the use of a suitable linker, such as an amino acid or polypeptide linker, e.g., comprising threonine, serine, proline, glycine, aspartic acid, lysine, glutamine, glutamic acid, asparagine, arginine, phenylalanine, or alanine, or a combination thereof. The EPO polypeptide can be linked to another molecule (e.g., a protein) by a thiol linkage (e.g., disulfide or thioether bond), or through an amine or alcohol group. Linkages of the EPO polypeptide to another molecule can be via any suitable linking moiety, such as an aryl azide, a maleimide, a carbodiimide, an N-hydroxysuccinimide ester, a hydrazide, a PFP-ester, a phosphine or hydroxymethyl phosphine, a psoralen, an imidoester, a pyridyl disulfide, an isocyanate, or a vinyl sulfone.

Without wishing to be bound by theory, it is believed that conjugating the EPO polypeptide to another molecule (e.g., a protein, such as albumin or an IgG molecule) can extend the serum half-life of the EPO polypeptide, provide ease of purification or isolation, modify (e.g., improve) secretion of the EPO polypeptide from a cell, provide an epitope tag, provide a detectable signal, and/or provide for dimerization or multimerization.

### Exemplary EPO Sequences

In an embodiment, the EPO polypeptide comprises an amino acid sequence adapted to achieve one or more (e.g., two, three, four, or all) of: (i) reduced binding affinity to a class II MHC allele (e.g., HLA-DRB1*09 allele) relative to a reference EPO polypeptide or preparation; (ii) maintained binding affinity to an EPO receptor; (iii) preserved structural integrity; (iv) maintained, or increased, serum half-life, relative to a reference EPO polypeptide or preparation; and/or (v) maintained, or improved bioactivity (e.g., as measured by a cell-proliferation assay, or by an increase in hemoglobin in a subject), relative to a reference EPO polypeptide or preparation. In an embodiment, the reference EPO polypeptide comprises, or consists of, a wild-type or naturally occurring EPO polypeptide, or a modified EPO polypeptide that lacks one or more (e.g., all) of the mutations described herein. In an embodiment, the reference EPO preparation is a BRP for EPO.

In an embodiment, an EPO polypeptide comprises an amino acid sequence provided in Table 2, or an amino acid sequence substantially identical thereto (e.g., an amino acid sequence at least about 85%, 90%, 91%, 92%, 93%, 94%, 95%, 96%, 97%, 98%, 99%, 99.5%, 99.6%, 99.7%, 99.8%, or 99.9% or more identical thereto), or a functional fragment thereto.

TABLE 2
Exemplary EPO Variants

| Name | Mutation | Amino Acid Sequence | Nucleotide Sequence |
|---|---|---|---|
| EPO- | — | SEQ ID NO: 1 | SEQ ID NO: 2 |
| WT | | APPRLICDSRVLERYLLEAK | ATGGAAACCGATACTCTGCTGCTCTGGGTCCTTCTGCTTTGGGTGCCTGGTTCGACTG |
| | | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GLRSLTTLLRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGCTCCGCAGCCTGACCACCCTGCTGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | L67V, | SEQ ID NO: 3 | SEQ ID NO: 4 |
| 1.1 | L102V | APPRLICDSRVLERYLLEAK | ATGGAAACCGATACTCTGCTGCTCTGGGTCCTTCTGCTTTGGGTGCCTGGTTCGACTG |
| | | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGVALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCGTGGCGCTGCTGTCCGAGGCCGTGCTCAGGGG |
| | | GVRSLTTLLRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGGTCCGCAGCCTGACCACCCTGCTGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | L70V, | SEQ ID NO: 5 | SEQ ID NO: 6 |
| 1.2 | V74L, | APPRLICDSRVLERYLLEAK | ATGGAAACCGATACTCTGCTGCTCTGGGTCCTTCTGCTTTGGGTGCCTGGTTCGACTG |
| | L102I | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGLALVSEALLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGGTGTCCGAGGCCCTGCTCAGGGG |
| | | GIRSLTTLLRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGATTCGCAGCCTGACCACCCTGCTTCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRIGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | L102T | SEQ ID NO: 7 | SEQ ID NO: 8 |
| 1.3 | | APPRLICDSRVLERYLLEAK | ATGGAAACCGATACTCTGCTGCTCTGGGTCCTTCTGCTTTGGGTGCCTGGTTCGACTG |
| | | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GTRSLTTLLRALGAQKEAIS | ACAAGCACTCCTCGTGAATAGCTCGCAGCCCTGGGAACCGCTGCAGCTGCACGTGGAT |
| | | PPDAASAAPLRTITADTERK | AAGGCTGTGTCAGGGACCCGCAGCCTGACCACCCTGCTGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | L67N, | SEQ ID NO: 9 | SEQ ID NO: 10 |
| 1.4 | V74N, | APPRLICDSRVLERYLLEAK | ATGGAAACCGATACTCTGCTGCTCTGGGTCCTTCTGCTTTGGGTGCCTGGTTCGACTG |
| | L102D | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGNALLSEANLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCAACGCGCTGCTGTCCGAGGCCAACCTCAGGGG |
| | | GDRSLTTLLRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGGATCGCAGCCTGACCACCCTGCTGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | L67D, | SEQ ID NO: 11 | SEQ ID NO: 12 |
| 1.5 | V74D, | APPRLICDSRVLERYLLEAK | ATGGAAACCGATACTCTGCTGCTCTGGGTCCTTCTGCTTTGGGTGCCTGGTTCGACTG |
| | L102N | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGDALLSEADLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCGATGCGCTGCTGTCCGAGGCCGACCTCAGGGG |
| | | GNRSLTTLLRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGAACCGCAGCCTGACCACCCTGCTGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | L12A, | SEQ ID NO: 13 | SEQ ID NO: 14 |
| 2.1 | Y15I, | APPRLICDSRVAERILLEAK | ATGGAAACCGATACTCTGCTGCTCTGGGTCCTTCTGCTTTGGGTGCCTGGTTCGACTG |
| | L105F, | EAENITTGCAEHCSLNENIT | GAGCTCCGCCTCGGTTGATCTGCGACTCCCGCGTGGCGGAAAGAATCCTCCTGGAAGC |
| | L149W | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GLRSFTTLLRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGCTCCGCAGCTTCACCACCCTGCTGCGGGCCTTGGGCGCGCAAA |
| | | LFRVYSNFWRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCTGGGGGGAAAGCTGAAG |
| | | | CTGTACACTGGAGAGGCCTGCAGAACGGGGGACCGCTAATGA |
| EPO- | L12I, | SEQ ID NO: 15 | SEQ ID NO: 16 |
| 2.2 | Y15W, | APPRLICDSRVIERWLLEAK | ATGGAAACCGATACTCTGCTGCTCTGGGTCCTTCTGCTTTGGGTGCCTGGTTCGACTG |
| | L105S, | EAENITTGCAEHCSLNENIT | GAGCTCCGCCTCGGTTGATCTGCGACTCCCGCGTGATCGAAAGATGGCTCCTGGAAGC |
| | L149V | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GLRSSTTLLRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGCTCCGCAGCTCCACCACCCTGCTGCGGGCCTTGGGCGCGCAAA |
| | | LFRVYSNFVRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCGTGCGGGGAAAGCTGAAG |
| | | | CTGTACACTGGAGAGGCCTGCAGAACGGGGGACCGCTAATGA |
| EPO- | L105F | SEQ ID NO: 17 | SEQ ID NO: 18 |
| 2.3 | | APPRLICDSRVLERYLLEAK | ATGGAAACCGATACTCTGCTGCTCTGGGTCCTTCTGCTTTGGGTGCCTGGTTCGACTG |
| | | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GLRSFTTLLRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGCTCCGCAGCTTCACCACCCTGCTGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | T106A | SEQ ID NO: 19 | SEQ ID NO: 20 |
| 3.1 | | APPRLICDSRVLERYLLEAK | ATGGAAACCGATACTCTGCTGCTCTGGGTCCTTCTGCTTTGGGTGCCTGGTTCGACTG |
| | | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GLRSLATLLRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGCTCCGCAGCCTGGCCACCCTGCTGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | T106G | SEQ ID NO: 21 | SEQ ID NO: 22 |
| 3.2 | | APPRLICDSRVLERYLLEAK | ATGGAAACCGATACTCTGCTGCTCTGGGTCCTTCTGCTTTGGGTGCCTGGTTCGACTG |
| | | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GLRSLGTLLRALGAQKEAIS | ACAAGCACTCCTCGTGAATAGCTCGCAGCCCTGGGAACCGCTGCAGCTGCACGTGGAT |
| | | PPDAASAAPLRTITADTERK | AAGGCTGTGTCAGGGCTCCGCAGCCTGGGCACCCTGCTGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | T106H | SEQ ID NO: 23 | SEQ ID NO: 24 |
| 3.3 | | APPRLICDSRVLERYLLEAK | ATGGAAACCGATACTCTGCTGCTCTGGGTCCTTCTGCTTTGGGTGCCTGGTTCGACTG |
| | | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GLRSLHTLLRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGCTCCGCAGCCTGCACACCCTGCTGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | L109A | SEQ ID NO: 25 | SEQ ID NO: 26 |
| 4.1 | | APPRLICDSRVLERYLLEAK | ATGGAAACCGATACTCTGCTGCTCTGGGTCCTTCTGCTTTGGGTGCCTGGTTCGACTG |
| | | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GLRSLTTLARALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGCTCCGCAGCCTGACCACCCTGGCGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | L109W | SEQ ID NO: 27 | SEQ ID NO: 28 |
| 4.2 | | APPRLICDSRVLERYLLEAK | ATGGAAACCGATACTCTGCTGCTCTGGGTCCTTCTGCTTTGGGTGCCTGGTTCGACTG |
| | | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GLRSLTTLWRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGCTCCGCAGCCTGACCACCCTGTGGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | L109C, | SEQ ID NO: 29 | SEQ ID NO: 30 |
| 4.3 | A114C | APPRLICDSRVLERYLLEAK | ATGGAAACCGATACTCTGCTGCTCTGGGTCCTTCTGCTTTGGGTGCCTGGTTCGACTG |
| | | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GLRSLTTLCRALGCQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGCTCCGCAGCCTGACCACCCTGTGTCGGGCCTTGGGCTGCCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | L102T, | SEQ ID NO: 31 | SEQ ID NO: 32 |
| 5.1 | L105S, | APPRLICDSRVLERYLLEAK | ATGGAAACCGATACTCTGCTGCTCTGGGTCCTTCTGCTTTGGGTGCCTGGTTCGACTG |
| | T106H, | EAENITTGCAEHCSLNENIT | GAGCTCCGCCTCGGTTGATCTGCGACTCCCGCGTGCTGGAAAGATACCTCCTGGAAGC |
| | L109W | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GTRSSHTLWRALGAQKEAIS | ACAAGCACTCCTCGTGAATAGCTCGCAGCCCTGGGAACCGCTGCAGCTGCACGTGGAT |
| | | PPDAASAAPLRTITADTERK | AAGGCTGTGTCAGGGACCCGCAGCTCCCACACCCTGTGGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | L102T, | SEQ ID NO: 33 | SEQ ID NO: 34 |
| 5.2 | L105S, | APPRLICDSRVLERYLLEAK | ATGGAAACCGATACTCTGCTGCTCTGGGTCCTTCTGCTTTGGGTGCCTGGTTCGACTG |
| | T106G, | EAENITTGCAEHCSLNENIT | GAGCTCCGCCTCGGTTGATCTGCGACTCCCGCGTGCTGGAAAGATACCTCCTGGAAGC |
| | L109W | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GTRSSGTLWRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGACCCGCAGCTCCGGCACCCTGTGGCGGGCCTTGGGCGCGCAAA |
| | | LFRVYSNFLRGKLKLYTGEA | AGGAAGCCATCTCCCCCCCGGACGCCGCCAGCGCAGCGCCACTGAGGACTATTACCGC |
| | | CRTGDR | CGACACATTCCGCAAGCTGTTTCGGGTGTACTCCAACTTCCTCCGGGGAAAGCTGAAG |
| | | | CTGTACACTGGAGAGGCCTGCAGAACGGGGGACCGCTAATGA |
| EPO- | S146N, | SEQ ID NO: 35 | SEQ ID NO: 36 |
| 6.1 | K152Q | APPRLICDSRVLERYLLEAK | ATGGAAACCGATACTCTGCTGCTCTGGGTCCTTCTGCTTTGGGTGCCTGGTTCGACTG |
| | | EAENITTGCAEHCSLNENIT | GAGCTCCGCCTCGGTTGATCTGCGACTCCCGCGTGCTGGAAAGATACCTCCTGGAAGC |
| | | VPDTKVNFYAWKRMEVGQQA | CAAAGAGGCCGAGAACATCACCACCGGTTGTGCCGAGCATTGCTCCCTGAACGAAAAC |
| | | VEVWQGLALLSEAVLRGQAL | ATTACCGTGCCCGACACCAAGGTCAACTTCTACGCTTGGAAGCGGATGGAAGTCGGCC |
| | | LVNSSQPWEPLQLHVDKAVS | AGCAGGCCGTGGAGGTCTGGCAGGGCCTGGCGCTGCTGTCCGAGGCCGTGCTCAGGGG |
| | | GLRSLTTLLRALGAQKEAIS | ACAAGCACTCCTCGTGAATAGCTCGCAGCCCTGGGAACCGCTGCAGCTGCACGTGGAT |
| | | PPDAASAAPLRTITADTFRK | AAGGCTGTGTCAGGGCTCCGCAGCCTGACCACCCTGCTGCGGGCCTTGGGCGCGCAAA |
| | | LFRVYNNFLRGQLKLYTGEA | AGGAAGCCATCTCCCCCCCGGACGCCGCCAGCGCAGCGCCACTGAGGACTATTACCGC |
| | | CRIGDR | CGACACATTCCGCAAGCTGTTTCGGGTGTACAACAACTTCCTCCGGGGACAGCTGAAG |
| | | | CTGTACACTGGAGAGGCCTGCAGAACGGGGGACCGCTAATGA |

In an embodiment, the EPO polypeptide comprises the amino acid sequence of SEQ ID NO: 1, or an amino acid sequence differing by no more than 1, 2, 5, 10, 15, or 20 amino acids from, or at least 85%, 90%, 95%, 98%, or 99% identical to, SEQ ID NO: 1, or a functional fragment thereof. In an embodiment, the EPO polypeptide comprises the amino acid sequence of SEQ ID NO: 1 or a functional fragment thereof. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 3, or a variant thereof differing by no more than 1, 2, 5, 10, 15, or 20 amino acids from, or at least 85%, 90%, 95%, 98%, or 99% identical to, SEQ ID NO: 3, or a functional fragment thereof. In an embodiment, the EPO polypeptide comprises the amino acid sequence of SEQ ID NO: 3. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 5, or a variant thereof differing by no more than 1, 2, 5, 10, 15, or 20 amino acids from, or at least 85%, 90%, 95%, 98%, or 99% identical to, SEQ ID NO: 5, or a functional fragment thereof. In an embodiment, the EPO polypeptide comprises the amino acid sequence of SEQ ID NO: 5. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 7, or a variant thereof differing by no more than 1, 2, 5, 10, 15, or 20 amino acids from, or at least 85%, 90%, 95%, 98%, or 99% identical to, SEQ ID NO: 7, or a functional fragment thereof. In an embodiment, the EPO polypeptide comprises the amino acid sequence of SEQ ID NO: 7. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 9, or a variant thereof differing by no more than 1, 2, 5, 10, 15, or 20 amino acids from, or at least 85%, 90%, 95%, 98%, or 99% identical to, SEQ ID NO: 9, or a functional fragment thereof. In an embodiment, the EPO polypeptide comprises the amino acid sequence of SEQ ID NO: 9. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 11, or a variant thereof differing by no more than 1, 2, 5, 10, 15, or 20 amino acids from, or at least 85%, 90%, 95%, 98%, or 99% identical to, SEQ ID NO: 11, or a functional fragment thereof. In an embodiment, the EPO polypeptide comprises the amino acid sequence of SEQ ID NO: 11. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 13, or a variant thereof differing by no more than 1, 2, 5, 10, 15, or 20 amino acids from, or at least 85%, 90%, 95%, 98%, or 99% identical to, SEQ ID NO: 13, or a functional fragment thereof. In an embodiment, the EPO polypeptide comprises the amino acid sequence of SEQ ID NO: 13. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 15, or a variant thereof differing by no more than 1, 2, 5, 10, 15, or 20 amino acids from, or at least 85%, 90%, 95%, 98%, or 99% identical to, SEQ ID NO: 15, or a functional fragment thereof. In an embodiment, the EPO polypeptide comprises the amino acid sequence of SEQ ID NO: 15. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 17, or a variant thereof differing by no more than 1, 2, 5, 10, 15, or 20 amino acids from, or at least 85%, 90%, 95%, 98%, or 99% identical to, SEQ ID NO: 17, or a functional fragment thereof. In an embodiment, the EPO polypeptide comprises the amino acid sequence of SEQ ID NO: 17. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 19, or a variant thereof differing by no more than 1, 2, 5, 10, 15, or 20 amino acids from, or at least 85%, 90%, 95%, 98%, or 99% identical to, SEQ ID NO: 19, or a functional fragment thereof. In an embodiment, the EPO polypeptide comprises the amino acid sequence of SEQ ID NO: 19. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 21, or a variant thereof differing by no more than 1, 2, 5, 10, 15, or 20 amino acids from, or at least 85%, 90%, 95%, 98%, or 99% identical to, SEQ ID NO: 21, or a functional fragment thereof. In an embodiment, the EPO polypeptide comprises the amino acid sequence of SEQ ID NO: 21. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 23, or a variant thereof differing by no more than 1, 2, 5, 10, 15, or 20 amino acids from, or at least 85%, 90%, 95%, 98%, or 99% identical to, SEQ ID NO: 23, or a functional fragment thereof. In an embodiment, the EPO polypeptide comprises the amino acid sequence of SEQ ID NO: 23. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 25, or a variant thereof differing by no more than 1, 2, 5, 10, 15, or 20 amino acids from, or at least 85%, 90%, 95%, 98%, or 99% identical to, SEQ ID NO: 25, or a functional fragment thereof. In an embodiment, the EPO polypeptide comprises the amino acid sequence of SEQ ID NO: 25. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 27, or a variant thereof differing by no more than 1, 2, 5, 10, 15, or 20 amino acids from, or at least 85%, 90%, 95%, 98%, or 99% identical to, SEQ ID NO: 27, or a functional fragment thereof. In an embodiment, the EPO polypeptide comprises the amino acid sequence of SEQ ID NO: 27. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 29, or a variant thereof differing by no more than 1, 2, 5, 10, 15, or 20 amino acids from, or at least 85%, 90%, 95%, 98%, or 99% identical to, SEQ ID NO: 29, or a functional fragment thereof. In an embodiment, the EPO polypeptide comprises the amino acid sequence of SEQ ID NO: 29. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 31, or a variant thereof differing by no more than 1, 2, 5, 10, 15, or 20 amino acids from, or at least 85%, 90%, 95%, 98%, or 99% identical to, SEQ ID NO: 31, or a functional fragment thereof. In an embodiment, the EPO polypeptide comprises the amino acid sequence of SEQ ID NO: 31. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 33, or a variant thereof differing by no more than 1, 2, 5, 10, 15, or 20 amino acids from, or at least 85%, 90%, 95%, 98%, or 99% identical to, SEQ ID NO: 33, or a functional fragment thereof. In an embodiment, the EPO polypeptide comprises the amino acid sequence of SEQ ID NO: 33. In an embodiment, the EPO polypeptide consists the amino acid sequence of SEQ ID NO: 1 or a functional fragment thereof.

In an embodiment, the EPO polypeptide comprises the amino acid sequence of SEQ ID NO: 35, or a variant thereof differing by no more than 1, 2, 5, 10, 15, or 20 amino acids from, or at least 85%, 90%, 95%, 98%, or 99% identical to, SEQ ID NO: 35, or a functional fragment thereof. In an embodiment, the EPO polypeptide comprises the amino acid sequence of SEQ ID NO: 35. In an embodiment, the EPO polypeptide consists the amino acid sequence of SEQ ID NO: 1 or a functional fragment thereof.

In an embodiment, the EPO polypeptide comprises the amino acid sequence of

(SEQ ID NO: 42)
APPRLICDSRVX₁₂ERX₁₅LLEAKEAENITTGCAEHCSLNENITVPDTKV
NFYAWKRMEVGQQAVEVWQGX₆₇ALX₇₀SEAX₇₄LRGQALLVNSSQPWEPL
QLHVDKAVSGX₁₀₂RSX₁₀₅X₁₀₆TLX₁₀₉RALGX₁₁₄QKEAISPPDAASAAPL
RTITADTFRKLFRVYX₁₄₆NFX₁₄₉RGX₁₅₂LKLYTGEACRTGDR,

wherein X₁₂ is L, A, or I; X₁₅ is Y, I, or W; X₆₇ is L, D, N, or V; X₇₀ is L or V; X₇₄ is V, D, L, or N; X₁₀₂ is L, D, I, N, T, or V; X₁₀₅ is L, F, or S; X₁₀₆ is T, A, G, or H; X₁₀₉ is L, A, C, or W; X₁₁₄ is A or C; X₁₄₆ is S or N; X₁₄₉ is L, V, or W; and X₁₅₂ is K or Q, with the proviso that the EPO polypeptide does not comprise SEQ ID NO: 1.

### Computational Design of EPO Variants

Another aspect of the present disclosure features methods to design EPO polypeptides that have lower immunogenicity, relative to a reference EPO polypeptide or preparation (e.g., wild-type or naturally occurring EPO, a BRP for EPO, or a modified EPO polypeptide that lacks one or more (e.g., all) of the mutations described herein). For example, computational studies can be carried out to identify sites in the human EPO amino sequence predicted to bind with one or more class II MHC alleles (e.g., HLA-DRB1*09 or HLA-DRB1*09-DQB1*03:09 allele). Using these predicted binding sites, EPO variants possessing one or more mutations at the binding sites can be designed to lower (e.g., eliminate) affinity of the binding of the EPO polypeptide variant to the class II MHC allele. The computational tool can also be used to design proteins that will have preserved structural integrity, e.g., to avoid reducing the bioactivity or serum half-life of the EPO polypeptide relative to a reference EPO polypeptide or preparation (e.g., wild-type or naturally occurring EPO, a BRP for EPO, or a modified EPO polypeptide that lacks one or more (e.g., all) of the mutations described herein). In an embodiment, an in silico tool (e.g., NetMHCII 2.2) is used to predict sites on EPO that favor binding to an MHCII allele (e.g., HLA-DRB1*09 allele). In an embodiment, an in silico tool (e.g., NetMHCII 2.2) is used to design EPO polypeptide sequences that will result in an EPO polypeptide with reduced immunogenicity, e.g., due to having a lower affinity to an MHCII allele (e.g., HLA-DRB1*09 allele), relative to a reference EPO polypeptide or preparation (e.g., wild-type or naturally occurring EPO, a BRP for EPO, or a modified EPO polypeptide that lacks one or more (e.g., all) of the mutations described herein).

### Expression and Purification of EPO Variants

The EPO polypeptides described herein can be produced, e.g., by recombinant expression in a host cell, e.g., a cell described herein, using known techniques. In an embodiment, expression of the EPO polypeptide involves preparing a suitable expression vector containing a sequence encoding the EPO polypeptide. The vector can be any vector described herein. For example, a pcDNA expression vector (e.g., pcDNA 3.3 expression vector), or a suitable alternative, containing a sequence encoding an EPO polypeptide can be used. Once generated, and optionally purified, the expression vector may then be transfected into the appropriate cell. Any suitable host cell can be used, and can be, inter alia, a eukaryotic cell, a bacterial cell, an insect cell, or a human cell. In an embodiment, the host cell is a human cell. In an embodiment, the host cell is a FreeStyle™ 293-F cell. In an embodiment, transfected cells are cultured, e.g., according to the requirements of the cell, to produce the EPO polypeptide. EPO polypeptide produced from the culture may then be isolated and purified using known techniques, e.g., using anion exchange chromatography.

### Bioactivity of EPO Variants

Erythropoietin (both natural and recombinant forms) can bind with and activate an EPO receptor (EPO-R), a protein found on the surface of EPO responding cells. Without wishing to be bound by theory, it is believed that in an embodiment, the binding and activation of EPO-R by the EPO polypeptide is involved in stimulating erythropoiesis. More specifically, the EPO polypeptide can bind to the extracellular domain of EPO-R, resulting in a conformational change of the receptor and triggering intracellular signaling events, namely phosphorylation of the receptor and activation of the JAK-STAT, RAS and P13 kinase pathways. These events can lead to the promotion and differentiation of erythroid progenitor cells into erythrocytes, stimulating hemoglobin C synthesis and increasing hematocrit levels.

EPO-R can be found in (e.g., on the surface of) erythroid cells as well as several types of non-erythroid cells, such as myeloid cells, lymphocytes, megakaryocytes, endothelial cells, mesangial cells, myocardial cells, smooth muscle cells, neural cells, prostate cells, and renal cells. As such, EPO is associated with several biological processes in addition to promoting erythropoiesis, including, inter alia, the development of new blood vessels, and other important processes involving the brain, ovary, oviduct, uterus and testis, and can affect tumors.

Due in part to structural variation and confirmation of the EPO polypeptide, in addition to the effect of different isoforms, the EPO polypeptide can have varied bioactivity. For example, an EPO polypeptide variant may have a different binding affinity and/or ability to activate EPO-R, and/or possess a different serum half-life, relative to an unmodified EPO polypeptide (e.g., wild-type or naturally occurring EPO).

Methods to measure the bioactivity of an EPO polypeptide, both in vitro, ex vivo, and in vivo, have been described. For example, the bioactivity of an EPO polypeptide can be determined using a cell proliferation assay, e.g., using a cell line that expresses EPO receptors. In an embodiment, the bioactivity of the EPO polypeptide is determined using an in vitro assay, e.g., involving a TF-1 cell proliferation assay. The TF-1 cell line, derived from a patient with erythroleukemia, can be stimulated and differentiated into mature red blood cells upon contact with EPO, and therefore provides a useful platform to screen the in vitro bioactivity of EPO polypeptides. In particular, this cell type exhibits EPO receptors on its surface and shows commitment to erythroid lineage and has an intact JAK2/STAT5 pathway, which mediates cell proliferation in response to various growth factors including EPO (see, e.g., Broudy, V. C. et al. PNAS (1988) 85:6513-6517; Chretien, S. et al. Blood (1994) 83:1813-1821).

The EPO polypeptides described herein may possess varying degrees of bioactivity, e.g., as determined by a TF-1 cell line assay. The bioactivity of an EPO polypeptide can be expressed in terms of relative bioactivity in comparison to wild-type EPO. In an embodiment, an EPO polypeptide described herein (e.g., EPO-1.2, EPO-3.1, EPO-3.2, EPO-3.3 and EPO-4.1 as shown in Table 2) exhibits the same, or substantially the same, in vitro bioactivity, as compared to a reference EPO polypeptide or preparation (e.g., wild-type or naturally occurring EPO, a BRP for EPO, or a modified EPO polypeptide that lacks one or more (e.g., all) of the mutations described herein). Without wishing to be bound by theory, it is believed that in an embodiment, the variation in bioactivity on TF-1 cells may due to the differences in protein sequences, overall protein conformation and/or glycosylation pattern. As described in more detail herein, the isoform pattern of EPO polypeptides can have an important role in bioactivity, potency and/or stability (see, e.g., Egrie, J. C. et. al. Oncology (Williston Park) (2002) 16:13-22). For example, the in vivo activity of non-glycosylated EPO is reduced, while a higher level glycosylation is believed to increase persistence of EPO, resulting in a prolonged serum half-life (see, e.g., Elliott, S. et al. Exp. Hematol. (2004) 32:1146-1155; Kaushik, S. et al. Protein Sci. (2011) 20:465-481; Li, H. et al. Curr Opin Biotechnol (2009) 20: 678-684; Sinclair, A. M. et al.. J. Pharm. Sci. (2005) 94:1626-1635; and Su, D., et al. Int J Hematol (2010) 91: 238-244). Without wishing to be bound by theory, it is believed that in an embodiment, the variation of isoform distribution and isoform abundance in each EPO polypeptides may also explain the variation of bioactivity among the EPO polypeptides described herein.

In an embodiment, the EPO polypeptide has substantially the same bioactivity relative to a reference EPO polypeptide or preparation (e.g., wild-type or naturally occurring EPO, a BRP for EPO, or a modified EPO polypeptide that lacks one or more (e.g., all) of the mutations described herein). In an embodiment, the EPO variant has greater (e.g., at least 0.5, 1, 2, 3, 4, 5, 6, 7, 8, 9, or 10-fold greater) bioactivity relative to a reference EPO polypeptide or preparation (e.g., wild-type or naturally occurring EPO, a BRP for EPO, or a modified EPO polypeptide that lacks one or more (e.g., all) of the mutations described herein). In an embodiment, the EPO variant has lower (e.g., no more than 5%, 10%, 15%, 20%, 25%, 30%, 35%, 40%, 45%, or 50% lower) bioactivity relative to a reference EPO polypeptide or preparation (e.g., a wild type or naturally occurring EPO polypeptide, a BRP for EPO, or a modified EPO polypeptide that lacks one or more (e.g., all) of the mutations described herein). In an embodiment, the EPO variant has substantially the same bioactivity as a reference EPO polypeptide or preparation (e.g., a wild type or naturally occurring EPO polypeptide, a BRP for EPO, or a modified EPO polypeptide that lacks one or more (e.g., all) of the mutations described herein), as determined by a method described herein, e.g., a cell proliferation assay (e.g., a TF-1 cell proliferation assay).

In an embodiment, the EPO polypeptide variant possesses at least 2% of the bioactivity relative to a reference EPO polypeptide or preparation (e.g., a wild type or naturally occurring EPO polypeptide, a BRP for EPO, or a modified EPO polypeptide that lacks one or more (e.g., all) of the mutations described herein), e.g., at least 3%, 4%, 5%, 6%, 8%, 10%, 12%, 14%, 16%, 18%, 20%, 22%, 24%, 26%, 28%, 30%, 35%, 40%, 45%, 50%, 55%, 60%, 70%, 80%, 90%, 95%, 99%, or at least 99.9% bioactivity relative to a reference EPO polypeptide or preparation (e.g., a wild type or naturally occurring EPO polypeptide, a BRP for EPO, or a modified EPO polypeptide that lacks one or more (e.g., all) of the mutations described herein). In an embodiment, the EPO polypeptide variant possesses between about 1-10%, between about 10-20%, between about 30-40%, between about 40-50%, between about 50-60%, between about 60-70%, between about 70-80%, between about 80-90%, between about 90-100%, between about 40-45%, between about 20-25%, between about 2-4%, or between about 5-7% bioactivity relative to a reference EPO polypeptide or preparation (e.g., a wild type or naturally occurring EPO polypeptide, a BRP for EPO, or a modified EPO polypeptide that lacks one or more (e.g., all) of the mutations described herein). In an embodiment, the EPO polypeptide variant possesses greater bioactivity relative to a reference EPO polypeptide or preparation (e.g., wild-type EPO), e.g., at least 1%, 2%, 3%, 4%, 5%, 10%, 15%, 20%, 40%, 60%, 80%, 100%, 150%, 200%, 300%, 400%, or 500% or greater bioactivity relative to a reference EPO polypeptide or preparation (e.g., a wild type or naturally occurring EPO polypeptide, a BRP for EPO, or a modified EPO polypeptide that lacks one or more (e.g., all) of the mutations described herein). In an embodiment, the EPO polypeptide variant has the same, or substantially the same, bioactivity relative to a reference EPO polypeptide or preparation (e.g., a wild type or naturally occurring EPO polypeptide, a BRP for EPO, or a modified EPO polypeptide that lacks one or more (e.g., all) of the mutations described herein).

In an embodiment, the EPO variant has an increased stability, e.g., as determined by an increased half-life in vivo or in vitro, relative to a reference EPO polypeptide or preparation (e.g., a wild type or naturally occurring EPO polypeptide, a BRP for EPO, or a modified EPO polypeptide that lacks one or more (e.g., all) of the mutations described herein). In an embodiment, the EPO variant possesses a greater serum half-life relative to a reference EPO polypeptide or preparation (e.g., a wild type or naturally occurring EPO polypeptide, a BRP for EPO, or a modified EPO polypeptide that lacks one or more (e.g., all) of the mutations described herein), e.g., about 1%, 2%, 3%, 4%, 5%, 10%, 15%, 20%, 40%, 60%, 80%, 100%, 150%, 200%, 300%, 400%, or 500% or greater serum-half life, relative to a reference EPO polypeptide or preparation (e.g., a wild type or naturally occurring EPO polypeptide, a BRP for EPO, or a modified EPO polypeptide that lacks one or more (e.g., all) of the mutations described herein).

### Immunogenicity of EPO Polypeptides

In one aspect, the present disclosure features EPO polypeptides comprising an amino acid sequence tailored to reduce (e.g., eliminate) an immune response in a subject, e.g., as compared to a reference EPO polypeptide or preparation (e.g., a wild type or naturally occurring EPO polypeptide, a BRP for EPO, or a modified EPO polypeptide that lacks one or more (e.g., all) of the mutations described herein).

The administration of exogenous proteins can sometimes stimulate the immune system in a subject, causing an immune reaction. For example, the administration of conventional rHuEPO treatment (e.g., administration of epoetin alfa) can result in an immune reaction, and the development of anti-rHuEPO neutralizing antibodies that may cross-react with endogenous EPO. The consequences of immunogenicity to EPO can be severe, and lead to pure red cell aplasia in the affected subject. Such adverse events have been linked to certain risk factors. In particular, genetic factors can greatly increase the risk of immunogenicity to rHuEPO, e.g., due to the expression of certain class II MHC-alleles in the subject, that have been linked with the risk of developing rHuEPO-induced pure red cell aplasia (PRCA). Additionally, other factors such as the pharmaceutical formulation of the rHuEPO can increase the risk of immunogenicity, e.g., due to protein aggregates that can trigger an immune reaction. Further, the frequency of administration can increase the risk of forming an immune reaction, placing EPO-dependent subjects (e.g., subjects with chronic kidney disease) at a higher risk of developing rHuEPO-induced disorders such as pure red cell aplasia.

Without wishing to be bound by theory, it is believed that in an embodiment, the cause of immunogenicity to EPO polypeptides can be caused by the recognition of certain EPO epitopes by MHC molecules displayed on the cell surface of APCs, that trigger an immunogenic antibody response in the subject. Certain class II MHC alleles, e.g., HLA-DRB1*09 allele, have been linked to a higher risk of developing rHuEPO-induced PRCA. For example, the HLA-DRB1*09 allele is expressed at a high frequency in the Thai population, who are at high risk of developing rHuEPO-induced pure red cell aplasia.

In an embodiment, the EPO polypeptide comprises an amino acid sequence tailored to avoid or elicit a low immune response in a subject possessing the HLA-DRB1*09 allele. In an embodiment, the EPO polypeptide has a reduced HLA-binding affinity (e.g., reduced HLA-DRB1*09 allele binding affinity), relative to a reference EPO polypeptide or preparation (e.g., a wild type or naturally occurring EPO polypeptide, a BRP for EPO, or a modified EPO polypeptide that lacks one or more (e.g., all) of the mutations described herein). In an embodiment, the EPO polypeptide has lower immunogenicity, relative to rHuEPO. In an embodiment, the EPO polypeptide is administered to a subject to reduce (e.g., eliminate) immunogenicity in the subject, relative to the immunogenicity elicited in a comparable subject administered with a reference EPO polypeptide or preparation (e.g., a wild type or naturally occurring EPO polypeptide, a BRP for EPO, or a modified EPO polypeptide that lacks one or more (e.g., all) of the mutations described herein). In an embodiment, the subject is susceptible to immunogenicity to rHuEPO, e.g., subjects possessing an HLA-DRB1*09 allele.

The immunogenicity of an EPO polypeptide can be determined using known methods. For example, an in vitro or ex vivo assay can be used, e.g., to measure the T-cell response elicited by an EPO polypeptide. In an embodiment, an ex vivo human primary model is used. For example, blood can be obtained from a subject (e.g., a subject known to be positive or negative for the HLA-DRB1*09 allele) and processed to obtain precursor dendritic cells and/or T-cells (e.g., CD4⁺ T-cells). In an embodiment, the precursor dendritic cells are converted to immature dendritic cells in a culture, and then contacted (e.g., pulsed) with the EPO polypeptide to obtain antigen presenting cells. In an embodiment, the dendritic cells contacted with the EPO polypeptide, and the T cells (e.g., CD4⁺ T-cells) obtained from the blood sample, are co-cultured. The dendritic cells and T-cells can be co-cultured at any appropriate ratio, e.g., at a ratio of about 1:40, 1:30, 1:20, or 1:10, respectively. In an embodiment, the dendritic cells and T-cells are co-cultured at a ratio of about 1:20, respectively. The T-cell response can be measured by quantifying the amount of interferon-gamma (IFN-γ) released from the co-culture. In an embodiment, the level of IFN-γ from coculture between unpulsed DC and CD4⁺ T cells is used as a background measure for T cell response. In an embodiment, T-cells are incubated with anti-CD3/anti-CD28 antibodies to obtain a positive control for activated T cell response. Statistical comparison of the relative T cell response of EPO polypeptide compared to a reference protein (e.g., wild type EPO, or a biological reference preparation (BRP)) may then be used to determine the relative immunogenicity of an EPO polypeptide.

In an embodiment, the EPO polypeptide elicits a reduced T-cell response relative to a reference EPO polypeptide or preparation (e.g., a wild type or naturally occurring EPO polypeptide, a BRP for EPO, or a modified EPO polypeptide that lacks one or more (e.g., all) of the mutations described herein). In an embodiment, the EPO exhibits a T-cell response, e.g., as measured by the magnitude of IFN-γ release, that is about 90% of the response elicited by a reference EPO polypeptide or preparation (e.g., a wild type or naturally occurring EPO polypeptide, a BRP for EPO, or a modified EPO polypeptide that lacks one or more (e.g., all) of the mutations described herein), e.g., about 80%, 70%, 60%, 50%, 40%, 30%, 20%, 10%, 5%, 4%, 3%, 2%, 1%, 0.5%, 0.4%, 0.3%, 0.2%, 0.1%, 0.05%, 0.04%, 0.03%, 0.02%, 0.01%, or less, relative to the T-cell response elicited by a reference EPO polypeptide or preparation (e.g., a wild type or naturally occurring EPO polypeptide, a BRP for EPO, or a modified EPO polypeptide that lacks one or more (e.g., all) of the mutations described herein), e.g., as determined by IFN-γ release.

In an embodiment, a reference EPO polypeptide or preparation (e.g., a wild type or naturally occurring EPO polypeptide, a BRP for EPO, or a modified EPO polypeptide that lacks one or more (e.g., all) of the mutations described herein) elicits a T-cell response about 1-fold greater than the T-cell response elicited by the EPO variant (e.g., an EPO variant described herein), e.g., about 2-fold, 5-fold, 10-fold, 100-fold, 1000-fold, 10,000-fold, or 100,000-fold greater than the T-cell response elicited by the EPO polypeptide.

In an embodiment, the EPO polypeptide elicits a T-cell response resulting in an IFN-γ release between about 100 to about 50,000 pg/mL, e.g., between about 150 to about 45,000 pg/mL, or between about 200 to about 40,000 pg/mL. In an embodiment, the EPO variant elicits a T-cell response resulting in an IFN-γ release of about 200, 220, 240, 260, 280, 300, 400, 500, 1000, 2000, 3000, 4000, 5000, 10,000, 15,000, 20,000, 25,000, 30,000, 35,000, or 40,000 pg/mL.

In an embodiment, the EPO polypeptide possesses one or more (e.g., 2, 3, 4, 5, 6, 7, 8, 9, 10, 11, 12, 13, 14, 15, 16, 17, 18, 19, 20, 21, 22, 23, 24, or all) amino acid mutations at positions 74, 75, 76, 77, 79, 80, 81, 82, 102, 105, 106, 109, 111, 112, 113, 114, 115, 116, 142, 145, 146, 148, 149, 152, or 153 in the amino acid sequence, resulting in a reduced immunogenicity, relative to a reference EPO polypeptide or preparation (e.g., a wild type or naturally occurring EPO polypeptide, a BRP for EPO, or a modified EPO polypeptide that lacks one or more (e.g., all) of the mutations described herein).

In an embodiment, the magnitude of immunogenicity observed in the EPO polypeptide is significantly decreased in HLA-DRB1*09-positive subjects when compared to HLA-DRB1*09-negative subjects. In an embodiment, the EPO polypeptide with reduced immunogenicity is associated with the reduced HLA-DRB1*09 binding affinity. In an embodiment, the mutation possessed by the EPO polypeptide is tailored to reduce the binding between EPO epitope and HLA-DRB1*09 paratope. In an embodiment, the EPO polypeptide with reduced immunogenicity exhibits a similar biopotency, and/or unaffected biological function, with respect to a wild-type EPO polypeptide.

### Animal Models

The EPO polypeptides described herein can be evaluated in vivo, e.g., using various animal models. For example, an animal model can be used to test the pharmacokinetic and/or pharmacodynamic properties of an EPO polypeptide described herein in activating the EPO receptor, and/or in treating or preventing a disorder described herein, e.g., anemia or an EPO-deficiency related disorder, e.g., pure red cell aplasia Animal models can also be used, e.g., to investigate for side effects, measure concentrations of EPO polypeptides in situ, or measure immunogenicity of an EPO polypeptide.

Exemplary animal models for an EPO-deficiency associated disorder described herein, that can be used for evaluating an EPO polypeptide described herein include, but are not limited to, rodents (e.g., mice or rats, e.g., Sprague-Dawley rats). For example, rat models for anemia associated with chronic kidney disease are described, e.g., in Landau, D. et al. PLoS One (2018) 13(5): e0196684, and rat models for rHuEPO-induced pure red cell aplasia (PRCA) are described, e.g., in Woodburn, K. W. et. al. Exp. Hematol. (2007) 35(8):1201-1208.

Exemplary animal models for disorders described herein are also known in the art. Exemplary types of animals that can be used to evaluate the EPO polypeptides described herein include, but are not limited to, mice, rats, rabbits, guinea pigs, dogs, and monkeys.

### Nucleic Acids and Vectors

In another aspect, the present disclosure provides nucleic acids comprising nucleotide sequences encoding an EPO polypeptide described herein. A nucleic acid described herein may comprise a nucleotide sequence encoding any one of the amino acid sequences described herein, or a sequence substantially identical thereto (e.g., a sequence at least about 85%, 90%, 95%, 99%, 99.5%, 99.9% or more identical thereto, or which differs by no more than 3, 6, 15, 30, or 45 nucleotides from the sequences described herein). In an embodiment, a nucleic acid encodes an EPO polypeptide described in Table 2, or a fragment thereof. In an embodiment, a nucleic acid described herein encodes EPO-1.1, EPO-1.2, EPO-1.3, EPO-1.4, EPO-1.5, EPO-2.1, EPO-2.2, EPO-2.3, EPO-3.1, EPO-3.2, EPO-3.3, EPO-4.1, EPO-4.2, EPO-4.3, EPO-5.1, EPO-5.2, or EPO-6.1, or a functional fragment thereof.

In an embodiment, the nucleic acid comprises a nucleotide sequence shown in Table 2. In an embodiment, the nucleic acid comprises a nucleotide sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, and SEQ ID NO: 36. In an embodiment, the nucleic acid encodes a protein comprising an amino acid sequence shown in Table 2. In an embodiment, the nucleic acid encodes a protein comprising an amino acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, and SEQ ID NO: 35.

A nucleic acid described herein may comprise a deoxyribonucleotide, a ribonucleotide, or an analog thereof. The nucleic acid can be a single-stranded or double-stranded, and if single-stranded, it may be the coding strand or non-coding (antisense) strand. In an embodiment, a polynucleotide comprises a modified nucleotide, e.g., a methylated nucleotide or nucleotide analog. A sequence of nucleotides in a nucleic acid described herein can be interrupted by non-nucleotide components. In an embodiment, a nucleic acid is further modified after polymerization, such as by conjugation with a labeling component. The nucleic acid may be a recombinant polynucleotide, or a polynucleotide of genomic, cDNA, semisynthetic, or synthetic origin. In an embodiment, a nucleic acid described herein is designed using computational methods (e.g., an in silico tool). In an embodiment, the nucleic acid is generated using gene synthesis and cloned into an expression vector (e.g., a pcDNA 3.3 expression vector) for transformation in a host cell.

The present disclosure additionally features vectors comprising a nucleotide sequence encoding an EPO polypeptide described herein. In an embodiment, the vector comprises a nucleotide sequence encoding an EPO polypeptide provided in Table 2. The vectors described herein include, but are not limited to, a virus, plasmid, cosmid, lambda phage, or a yeast artificial chromosome (YAC). In an embodiment, the vector is an expression vector, e.g., a pcDNA™ expression vector, e.g., pcDNA™ 3.3. Numerous vector systems can be employed, e.g., vectors that utilize DNA elements derived from animal viruses such as cytomegalovirus, bovine papilloma virus, polyoma virus, adenovirus, vaccinia virus, baculovirus, retrovirus (e.g., Rous Sarcoma Virus, MMTV, or MOMLV), or SV40 virus. RNA elements derived from RNA viruses may be utilized by a vector described herein, such as Semliki Forest virus, Eastern Equine Encephalitis virus, and flaviviruses.

Further, markers may be introduced which allow for the selection of cells which have stably integrated the DNA into their chromosomes (i.e., transfected host cells). The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Expression vectors or nucleic acid sequences (e.g., DNA sequences) containing the constructs may be transfected or introduced into an appropriate host cell. Various transfection techniques may be employed e.g., protoplast fusion, calcium phosphate precipitation, electroporation, retroviral transduction, viral transfection, gene gun, lipid based transfection or other conventional techniques. In the case of protoplast fusion, the cells may be grown in media and screened for the appropriate activity. Methods and conditions for culturing the resulting transfected cells and for recovering the desired protein (e.g., EPO polypeptide) are known to those skilled in the art, and may be varied or optimized depending upon the specific expression vector and mammalian host cell employed, based upon the present description.

### Cells

In another aspect, the present disclosure features cells (e.g., host cells) comprising a nucleic acid encoding an EPO polypeptide described herein. In an embodiment, the host cells comprise a nucleic acid encoding an amino acid sequence provided in Table 2, or a sequence substantially homologous thereto (e.g., a sequence at least about 80%, 85%, 90%, 95%, or 99% or more identical thereto), or a portion of one of said sequences. In an embodiment, the host cell is genetically engineered to comprise a nucleic acid encoding an EPO polypeptide described herein. In an embodiment, the host cell is genetically engineered to comprise a nucleic acid encoding EPO-1.1, EPO-1.2, EPO-1.3, EPO-1.4, EPO-1.5, EPO-2.1, EPO-2.2, EPO-2.3, EPO-3.1, EPO-3.2, EPO-3.3, EPO-4.1, EPO-4.2, EPO-4.3, EPO-5.1, EPO-5.2, or EPO-6.1, or a functional fragment thereof.

In an embodiment, the host cell is genetically engineered using an expression cassette. The phrase “expression cassette,” refers to nucleotide sequences, which are capable of effecting expression of a gene in a host cell compatible with such sequences. An expression cassette may include a promoter, an open reading frame with or without introns, and a termination signal. Additional factors necessary or helpful in effecting expression may also be used, such as, for example, an inducible promoter. In an embodiment, the host cell comprises a vector described herein.

The cell (e.g., host cell) can be, inter alia, a eukaryotic cell, a bacterial cell, an insect cell, or a human cell. Suitable eukaryotic cells include, inter alia, Vero cells, HeLa cells, COS cells, CHO cells, HEK293 cells, BHK cells and MDCKII cells. Suitable insect cells include, but are not limited to, Sf9 cells. Suitable bacterial cells include, inter alia, Escherichia coli (E. coli) cells. In an embodiment, the cell (e.g., host cell) is an isolated cell. In an embodiment, the cell (e.g., host cell) is an E. coli cell. In an embodiment, the cell (e.g., host cell) is a mammalian cell (e.g., a human cell). In an embodiment, the cell (e.g., host cell) is a human cell. In an embodiment, the cell (e.g., host cell) is derived from primary embryonal human kidney. In an embodiment, the cell (e.g., host cell) is a FreeStyle 293-F cell.

### Use of EPO Variants

The EPO polypeptides disclosed herein, as well as the pharmaceutical compositions disclosed herein, have in vitro, ex vivo, and in vivo therapeutic, prophylactic, and/or diagnostic utilities.

In an embodiment, the EPO polypeptide modulates (e.g., activates) one or more EPO receptors, e.g., to stimulate erythropoiesis. For example, these EPO polypeptides can be administered to cells in culture, in vitro or ex vivo, or to a subject, e.g., a human subject, e.g., in vivo, to modulate (e.g., activate) one or more EPO receptors. In an embodiment, the EPO polypeptide activates, or substantially activates, the EPO receptor, e.g., human EPO receptor to stimulate erythropoiesis, e.g., to form erythrocytes, stimulate hemoglobin C synthesis, and/or increase hematocrit levels. Accordingly, in an aspect, the disclosure provides a method of treating or preventing a disorder, e.g., a disorder described herein (e.g., anemia), in a subject, comprising administering to the subject an EPO polypeptide described herein, such that the disorder is treated or prevented. For example, the disclosure provides a method comprising contacting an EPO polypeptide described herein with cells in culture, e.g., in vitro or ex vivo, or administering an EPO polypeptide described herein to a subject, e.g., in vivo, to treat or prevent a disorder, e.g., a disorder described herein, e.g., a disorder associated with EPO (e.g., an EPO deficiency), or an anemia, e.g., anemia or a disorder associated with EPO (e.g., an EPO deficiency), e.g., as described herein.

In an embodiment, the EPO polypeptide has a reduced binding affinity for one or more class II MHC alleles (e.g., HLA-DRB1*09), relative to a reference EPO polypeptide or preparation (e.g., a wild type or naturally occurring EPO polypeptide, a BRP for EPO, or a modified EPO polypeptide that lacks one or more (e.g., all) of the mutations described herein). For example, the binding affinity for one or more class II MHC alleles (e.g., HLA-DRB1*09) is reduced by about 10%, 20%, 30%, 40%, 50%, 60%, 70%, 80%, 90%, 95%, or more, relative to the reference EPO polypeptide (e.g., a wild type or naturally occurring EPO polypeptide, a BRP for EPO, or a modified EPO polypeptide that lacks one or more (e.g., all) of the mutations described herein). In an embodiment, the EPO polypeptide has no binding affinity for one or more class II MHC alleles (e.g., HLA-DRB1*09). For example, these EPO polypeptides can be administered to immune cells (e.g., antigen presenting cells and/or T cells) in culture, in vitro or ex vivo, or to a subject, e.g., a human subject, e.g., in vivo, without inducing an immune response, or inducing an immune response that is reduced in comparison to a reference EPO polypeptide or preparation (e.g., a wild type or naturally occurring EPO polypeptide, a BRP for EPO, or a modified EPO polypeptide that lacks one or more (e.g., all) of the mutations described herein). Accordingly, in an aspect, the disclosure provides a method of treating or preventing a disorder, e.g., a disorder described herein (e.g., pure red cell aplasia), in a subject, comprising administering to the subject an EPO polypeptide described herein. For example, the disclosure provides a method comprising contacting an EPO polypeptide described herein with cells in culture, e.g., in vitro or ex vivo, or administering an EPO polypeptide described herein to a subject, e.g., in vivo, to treat or prevent a disorder, e.g., a disorder described herein, e.g., a disorder associated with EPO (e.g., an EPO deficiency), e.g., pure red cell aplasia.

As used herein, the term “subject” is intended to include human and non-human animals. In an embodiment, the subject is a human subject, e.g., a human patient having an anemia or a disorder associated with EPO (e.g., an EPO deficiency), or at risk of having an anemia, or a disorder associated with EPO (e.g., an EPO deficiency, e.g., as described herein. The term “non-human animals” includes mammals and non-mammals, such as non-human primates. In an embodiment, the subject is a human The methods and compositions described herein are suitable for treating human patients with an anemia or a disorder associated with EPO (e.g., an EPO deficiency) described herein. Patients having an anemia or a disorder associated with EPO (e.g., an EPO deficiency) described herein, include those who have developed an anemia or a disorder associated with EPO (e.g., an EPO deficiency) described herein, but are (at least temporarily) asymptomatic, patients who have exhibited a symptom of anemia or a disorder associated with EPO (e.g., an EPO deficiency) described herein, or patients having a disorder related to or associated with anemia or a disorder associated with EPO (e.g., an EPO deficiency) described herein.

In an embodiment, the EPO associated disorder (e.g., EPO deficiency associated disorder) is pure red cell aplasia (PRCA), e.g., antibody-positive PRCA.

In an embodiment, the subject has, or is at risk of having, an anemia. In an embodiment, the subject is treated for an anemia. In an embodiment, the subject has, or is at risk of having, pure red cell aplasia (PRCA), e.g., antibody-positive PRCA. In an embodiment, the subject is treated for pure red cell aplasia (PRCA), e.g., antibody-positive PRCA.

### Methods of Treating or Preventing Disorders

The EPO polypeptides (e.g., EPO variants) described herein can be used to treat or prevent disorders associated with EPO, e.g., anemias and/or disorders associated with EPO-deficiencies, or symptoms thereof.

In an embodiment, the disorder is associated with an EPO deficiency. In an embodiment, the disorder is associated or mediated by EPO. In an embodiment, the EPO polypeptide is used to treat a subject having a disorder described herein or is at risk of developing a disorder described herein.

Exemplary disorders, e.g., disorders associated with, or mediated by, EPO, include, but are not limited to, an anemia (e.g., anemia associated with renal failure; chronic kidney disease; chronic inflammation; cancer; HIV or AIDS; chemotherapy (e.g., cis-platinum induced anemia) AIDS or HIV therapy (e.g., AZT treatment); radiotherapy; and bone marrow transplantation), pure red cell aplasia (PRCA), iron storage disorders, aplitic anemias, excessive blood cell destruction, excessive blood loss, thalassemia, sickle cell disease, paroxysmal nocturnal hemoglobinuria, cystic fibrosis, diabetic nephropathy, sepsis, cerebral hypoxia or ischemia, rheumatic disease, myelodysplastic syndrome, congestive heart failure, Gaucher's disease, or Castleman's disease.

The EPO polypeptides described herein are typically administered at a frequency that keeps a therapeutically effective level and/or activity of EPO polypeptide in the patient's system until the patient recovers. In an embodiment, the EPO polypeptide is administered at a frequency that keeps a therapeutically effective level of EPO polypeptide in the patient's system for the duration of the patient's life. In an embodiment, the EPO polypeptide is administered at a frequency that maintains a sufficient level of hemoglobin in the patient's blood (e.g., greater than 10 g/dL, e.g., greater than 11, 12, 13, 14, or 15 g/dL, e.g., between about 12 to 15.5 g/dL in women, or between about 13.5 to about 17.5 g/dL in men). In an embodiment, the EPO polypeptides are administered every 1, 2, 3, 4, 5, 6, or 7 days, every 1, 2, 3, 4, 5, or 6 weeks, or every 1, 2, 3, 4, 5, or 6 months.

Methods of administering various EPO polypeptides are known in the art and are described below. Suitable dosages of the EPO polypeptides used will depend on the age and weight of the subject and the particular composition used.

In an embodiment, the EPO polypeptide is administered to the subject (e.g., a human subject) intravenously. In an embodiment, the EPO polypeptide is administered to the subject (e.g., a human subject) subcutaneously. In an embodiment, the EPO polypeptide is administered to the subject at a dose between about 10 units/kg and 500 units/kg, e.g., between about 15 units/kg and 400 units/kg, between about 20 units/kg and 350 units/kg, between about 25 units/kg and 350 units/kg, between about 50 units/kg and 300 units/kg, between about 75 units/kg and 300 units/kg, between about 100 units/kg and 300 units/kg, between about 100 units/kg and 250 units/kg, between about 150 units/kg and 250 units/kg, between about 50 units/kg and 150 units/kg, or between about 50 units/kg and 100 units/kg. In an embodiment, the EPO polypeptide is administered to the subject at a fixed dose between about 1000 units and 60,000 units, e.g., between about 2000 units and 40,000 units, between about 3000 units and 40,000 units, between about 4000 units and 40,000 units, between about 10,000 units and 40,000 units, between about 20,000 units and 30,000 units, or between about 30,000 units and 40,000 units. In an embodiment, the EPO polypeptide is administered once a week, twice a week, thrice a week, once every two weeks, once every three weeks, once every four weeks, once every eight weeks, once a month, once every two months, once every three months, or once every six months.

### Pure Red Cell Aplasia (PRCA)

The EPO polypeptides described herein can be used to treat or prevent pure red cell aplasia (PRCA).

PRCA is a type of anemia associated with several causes including virus infection, immunological mediation, pregnancy, pre-stage malignancy, toxic exposure, and drug effects. Features of PRCA symptoms generally include very low reticulocyte and erythroid progenitor cells, while other blood cell parameters are relatively normal. PRCA associated with rHuEPO has distinct features including, e.g., severe rHuEPO resistance, blood transfusion dependence, high serum ferritin, bone marrow showing the absence of red cell precursor and presence of anti-rHuEPO antibody (see, e.g., Means, R. T., Jr. Blood (2016) 128:2504-2509; Pollock, C. et al. Clin J Am Soc Nephrol (2008) 3:193-199). The incidence of EPO-associated PRCA is a significant burden on the affected population worldwide, especially in countries such as Thailand (see, e.g., Praditpornsilpa, K. et al. Nephrol Dial Transplant (2009) 24: 1545-1549).

The number of reported cases of PRCA due to the development of neutralizing antibodies against endogenous EPO and recombinant erythropoiesis-stimulating agents (ESAs) has increased worldwide (see, e.g., Asari A. et al. J. Am. Soc. Nephrol. (2004) 15:2204-2207; Boven K. et al. Kidney Int. (2005) 67:2346-2353; Casadevall, N. et al. N. Engl. J. Med. (2002) 346:469-475; Howman, R. Nephrol. Dial. Transplant (2007) 22:1462-1464; Kruger, A. Nephrol. Dial. Transplant (2003) 18:1033-1034; Lacreta, G. J. Bras. Nefrol. (2019) 41:145-151; and Sia, C. Ann Acad Med Singapore (2020) 49:46-48). These cases of PRCA are particularly common in patients receiving rHuEPO for the treatment of CKD-related anemia. Without wishing to be bound by theory, it is believed that while the development of autoantibodies against endogenous EPO in patients who have never been treated with ESAs is rare, anti-rHuEPO neutralizing antibodies may cross-react with endogenous EPO.

Without wishing to be bound by theory, in an embodiment, the immunogenic antibody response activated by rHuEPO is believed to be T-cell dependent (see, e.g., Pollock, C. vide supra; Mazor, R., et al. Am J Pathol (2018) 188:1736-1743), involving the steps of (i) internalization of antigen by professional antigen-presenting cells (APCs) such as dendritic cells (DCs); (ii) processing of the antigen into peptides (epitopes) by the mature APC and presentation to naïve T-cells by major histocompatibility complex (MHC) class II molecules on the APC surface; (iii) interaction of a T-cell receptor (TCR) with the MHC II-epitope complex; (iv) activation of the T-cell to release cytokines for B-cell activation; (v) recognition of the antigen bound via a specific T-cell epitope/MHC:TCR interaction by B-cells; (vi) delivery of cytokines by the T-cell and stimulation of the B-cell to proliferate and mature toward a plasma cell; and (vii) production of antibodies after clonal expansion and differentiation of B-cells into antibody-secreting plasma cells.

A study on the distribution of gene frequency of HLA-A, —B, -DR and -DQ alleles in Thai patients having a proven anti-rHuEPO associated PRCA, compared to the gene frequency of HLA alleles in subjects on a kidney transplant waiting list (e.g., subjects with chronic kidney disease) and subjects on the national stem cell donor registry (e.g., normal population), demonstrated an association between the HLA-DRB1*09 and the risk of anti-rHuEPO-associated PRCA. The study also showed that all subjects with PRCA that expressed the HLA-DRB1*09 allele also expressed the HLA-DQB1*03:09 allele (see, e.g., Praditpornsilpa, vide supra). Without wishing to be bound by theory, it is believed that the higher frequency of DRB1*09 allele in the Thai population (approximately 10 times higher than other regions) may contribute to the higher prevalence of anti-rHuEPO-associated PRCA.

### Pharmaceutical Compositions and Kits

In another aspect, the present disclosure features compositions, e.g., pharmaceutically acceptable compositions, which include an EPO polypeptide described herein, formulated together with a pharmaceutically acceptable carrier.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion). In an embodiment, less than about 5%, e.g., less than about 4%, 3%, 2%, or 1% of the EPO polypeptides in the pharmaceutical composition are present as aggregates. In an embodiment, the level of aggregates is determined by chromatography, e.g., high performance size exclusion chromatography (HP-SEC).

The compositions set out herein can be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In an embodiment, the EPO polypeptide is administered by intravenous infusion or injection. In an embodiment, the EPO polypeptide is administered by subcutaneous injection. In an embodiment, the EPO polypeptide is administered by intramuscular injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high protein (e.g., EPO polypeptide) concentration. Sterile injectable solutions can be prepared by incorporating the active compound (e.g., EPO polypeptide) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The EPO polypeptides described herein can be administered by a variety of methods. Several are known in the art, and for many therapeutic, prophylactic, or diagnostic applications, an appropriate route/mode of administration is intravenous injection or infusion, or subcutaneous injection. As will be appreciated by the skilled artisan, the route and/or mode of administration will vary depending upon the desired results. In an embodiment, the EPO polypeptide can be prepared with a carrier that will protect the protein against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In an embodiment, an EPO polypeptide can be orally administered, for example, with an inert diluent or an assimilable edible carrier. The EPO polypeptide (and other ingredients, if desired) may also be enclosed in a hard or soft shell gelatin capsule, compressed into tablets, or incorporated directly into the subject's diet. For oral therapeutic administration, the EPO polypeptide can be incorporated with excipients and used in the form of ingestible tablets, buccal tablets, troches, capsules, elixirs, suspensions, syrups, wafers, and the like. To administer an EPO by other than parenteral administration, it can be necessary to coat the protein with, or co-administer the protein with, a material to prevent its inactivation. Therapeutic, prophylactic, or diagnostic compositions can also be administered with medical devices, and several are known in the art. In an embodiment, an EPO polypeptide is administered in a composition comprising one or more of a binding agent, filler, lubricant, disintegrant, wetting agent, suspending agent, emulsifying agent, non-aqueous vehicle, and preservative, e.g., maltose or magnesium stearate.

Dosage regimens can be adjusted to provide the desired response (e.g., a therapeutic, prophylactic, or diagnostic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the EPO polypeptide and the particular therapeutic, prophylactic, or diagnostic effect to be achieved, and (b) the limitations inherent in the art of compounding such an EPO polypeptide for the treatment of sensitivity in individuals.

An exemplary, non-limiting range for a therapeutically, prophylactically, or diagnostically effective amount of an EPO polypeptide is about 10 units/kg and 500 units/kg body weight of a subject, e.g., between about 15 units/kg and 400 units/kg, between about 20 units/kg and 350 units/kg, between about 25 units/kg and 350 units/kg, between about 50 units/kg and 300 units/kg, between about 75 units/kg and 300 units/kg, between about 100 units/kg and 300 units/kg, between about 100 units/kg and 250 units/kg, between about 150 units/kg and 250 units/kg, between about 50 units/kg and 150 units/kg, or between about 50 units/kg and 100 units/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed compositions.

The pharmaceutical compositions herein may include a “therapeutically effective amount” or “prophylactically effective amount” of an EPO polypeptide described herein.

A “therapeutically effective amount” refers to an amount effective, at dosages and for periods of time necessary, to achieve the desired therapeutic result. A therapeutically effective amount of the EPO polypeptide may vary according to factors such as the disease state, age, sex, and weight of the individual, and the ability of the EPO polypeptide to elicit a desired response in the individual. A therapeutically effective amount is also one in which any toxic or detrimental effect of the EPO polypeptide is outweighed by the therapeutically beneficial effects. A “therapeutically effective dosage” typically increase a measurable parameter by at least about 5%, e.g., by at least about 10%, by at least about 20%, by at least about 30%, by at least about 40%, by at least about 60%, or by at least about 80% relative to untreated subjects. The measurable parameter can be, e.g., hemoglobin levels. For example, an EPO polypeptide described herein can be administered to achieve a hemoglobin level of greater than about 10 g/dL, e.g., greater than about 11 g/L, about 12 g/L, about 13 g/L, about 14 g/L, about 15 g/L, about 16 g/L, or about 17 g/L or more. The ability of an EPO polypeptide to increase a measurable parameter can be evaluated in an animal model system predictive of efficacy in treating or preventing anemia. Alternatively, this property of a composition can be evaluated by examining the ability of the EPO polypeptide to activate the EPO receptor, e.g., by an in vitro assay, e.g., by measuring TF-1 cell proliferation.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within this disclosure is a kit that comprises an EPO polypeptide, described herein. The kit can include one or more other elements including: instructions for use; other reagents, e.g., a label, a therapeutic agent, or an agent useful for chelating, or otherwise coupling, an EPO polypeptide to a label or therapeutic agent, or a radioprotective composition; devices or other materials for preparing the EPO polypeptide for administration; pharmaceutically acceptable carriers; and devices or other materials for administration to a subject.

### Combination Therapies

The EPO polypeptide described herein can be used in combination with other therapies. For example, the combination therapy can include an EPO polypeptide co-formulated with, and/or co-administered with, one or more additional therapeutic agents, e.g., one or more additional therapeutic agents described herein. In other embodiments, the EPO polypeptides are administered in combination with other therapeutic treatment modalities, e.g., other therapeutic treatment modalities described herein. Such combination therapies may advantageously utilize lower dosages of the administered therapeutic agents, thus avoiding possible toxicities or complications associated with the various monotherapies.

Administered “in combination”, as used herein, means that two (or more) different treatments are delivered to the subject before, or during the course of the subject's affliction with a disorder. In an embodiment, two or more treatments are delivered prophylactically, e.g., before the subject has the disorder or is diagnosed with the disorder. In another embodiment, the two or more treatments are delivered after the subject has developed or diagnosed with the disorder. In an embodiment, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In an embodiment of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In an embodiment, delivery is such that the reduction in a symptom, or other parameter related to the disorder is greater than what would be observed with one treatment delivered in the absence of the other. The effect of the two or more treatments can be partially additive, wholly additive, or greater than additive. In an embodiment, the effect of the two or more treatments can be synergistic. The delivery can be such that an effect of the first treatment delivered is still detectable when the second is delivered.

In an embodiment, the additional agent is a second EPO polypeptide, e.g., an EPO polypeptide different from a first EPO polypeptide. Exemplary EPO polypeptides that can be used in combination include, but are not limited to, any combination of the EPO polypeptides described herein, e.g., as listed in Table 2.

In an embodiment, the EPO polypeptide is administered in combination with a second therapy to treat or prevent a disease or disorder associated with, or mediated by, EPO, e.g., an anemia (e.g., an anemia associated with renal failure, chronic inflammation, cancer, or HIV or AIDS), thalassemia, sickle cell disease, an anemia associated with chemotherapy (e.g., cis-platinum induced anemia), an anemia associated with radiotherapy, an anemia associated with bone marrow transplantation, iron storage disorders, aplastic anemia, excessive blood cell destruction, excessive blood loss, an anemia associated with AIDS or HIV therapy (e.g., AZT treatment), paroxysmal nocturnal hemoglobinuria, cystic fibrosis, diabetic nephropathy, sepsis, cerebral hypoxia or ischemia, rheumatic disease, myelodysplastic syndrome, congestive heart failure, Gaucher's disease, or Castleman's disease.

Exemplary therapies that can be used in combination with an EPO polypeptide or a composition described herein can include, e.g., hemoglobin, supplemental iron (e.g., elemental iron; ferric salts or ferrous salts or compositions thereof, e.g., ferrous sulfate, ferrous gluconate, ferrous fumarate; iron dextran; heme iron polypeptide e.g., PROFERRIN®), colony stimulating factors including cytokines, lymphokines, interleukins, and hematopoietic growth factors (e.g., interleukin-10 (IL-1), interleukin-2 (IL-2), interleukin-3 (IL-3), interleukin-4 (IL-4), interleukin-5 (IL-5), interleukin-6 (IL-6), interleukin-9 (IL-9), interferon-β (IFN-β), interferon gamma (IFN-γ), granulocyte-colony stimulating factor (GM-CSF), granulocyte macrophage-colony stimulating factor (GM-CSF), macrophage-colony stimulating factor (M-CSF), thrombopoietin (TPO), leukemia inhibitory factor (LIF), stem cell factor (SCF), oncostatin M (OSM), and vascular endothelial growth factor (VEGF)). Additionally, the EPO polypeptide or composition described herein can be used in combination with another EPO polypeptide, e.g., a recombinant EPO polypeptide. Non-limiting examples of recombinant EPO polypeptides includes epoetin alfa (e.g., EPOGEN®, EPOADE®, EPOPEN®, EPDXITIN®, EPREX®, ERYPRO®, ESPO®, GLOBUREN®, PROCRIT®), epoetin beta (e.g., EPOCH®, EPOGIN®, ERANTIN®, ERITROGEN®, MAROGEN®, NEORECORMON®, RECORMON®), epoetin omega (e.g., EPOMAX®, HEMAX®), epoetin delta (e.g., DYNEPO®), and darbepoetin alfa (e.g., ARANESP®). The EPO polypeptide can also be combined with a continuous erythropoietin receptor activator (CERA) or synthetic erythropoiesis protein (SEP). In an embodiment, the EPO polypeptide is used in combination with another therapy such as a chemotherapeutic agent, e.g., a hormone, a photosensitizing agent, a radionuclide, a toxin, an anti-metabolite, a signaling modulator, an antibody (e.g., anticancer antibodies), a peptide or oligopeptide (e.g., an anticancer oligopeptide), an oligonucleotide (e.g., an anticancer oligonucleotide), or an angiogenesis inhibitor.

### EXAMPLES

In order that the disclosure described herein can be more fully understood, the following examples are set forth. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

In the following Examples, computational design was used to screen for immunogenic hotspots, for example, recognized by HLA-DRB1*09, and predicted seventeen mutants having anywhere between one through four mutations that reduce affinity for the allele, without disrupting the structural integrity and bioactivity. Five out of seventeen mutants were found to be less immunogenic in vitro while retaining similar or slightly reduced bioactivity than a reference recombinant human EPO (rHuEPO). These engineered proteins can be used to treat patients who are rHuEPO-dependent and express the HLA-DRB1*09 allele.

### Example 1: Design of Recombinant Human EPO Variants

### MHC Epitope Prediction Analysis

A computational assessment was carried out to predict mutations of the erythropoietin (EPO) amino acid sequence that would disrupt binding with the MHC II allele, while preserving the protein structure and binding with the EPO receptor. Specifically, an in silico tool (NetMHCII 2.2) was used to predict amino acid residues responsible for binding between EPO epitopes and major histocompatibility complex (MHC) class II allele HLA-DRB1*09-DQB1*03:09 (Jensen et al. Immunology (2018), 154:394-406). The five core binding sites were predicted to be VLRGQALLV (SEQ ID NO: 37) (position 74-82), LRSLTTLLR (SEQ ID NO: 38) (position 102-110), LLRALGAQK (SEQ ID NO: 39) (position 108-116), FRVYSNFLR (SEQ ID NO: 40) (position 142-150), and YSNFLRGKL (SEQ ID NO: 41) (position 145-153), which mapped to 3 distinct non-overlapping regions (residues 74-82 (VLRGQALLV (SEQ ID NO: 37)), 102-116 (LRSLTTLLRALGAQK (SEQ ID NO: 44)), and 142-153 (FRVYSNFLRGKL (SEQ ID NO: 45))).

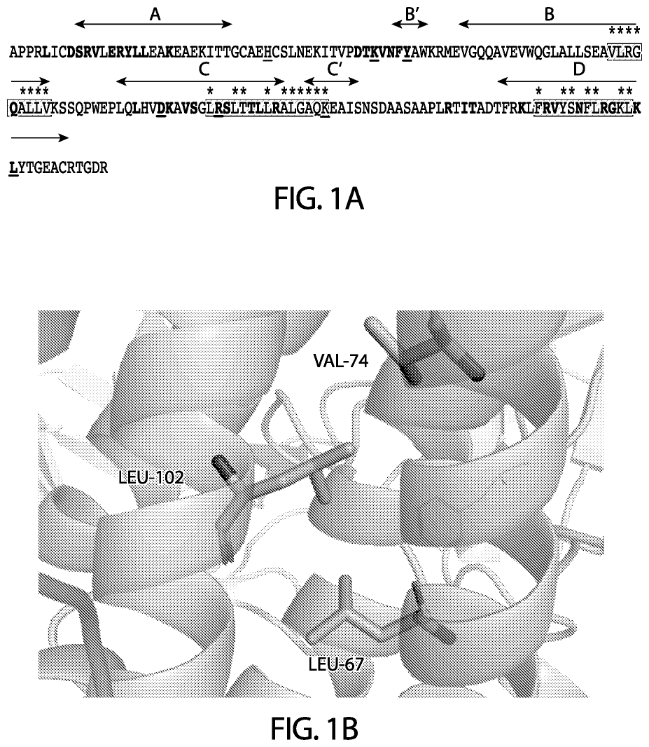

The predicted alleles were overlapping with the sites recognized by the two cell-surface erythropoietin receptors, inferred from the co-crystal structure (PDB: lEER). Known sites of neutralizing anti-EPO antibodies from the literature were also mapped. For example, FIG. 1A provides the amino acid sequence of EPO, indicating sites contacted by the receptor (PDB: lEER) are marked in bold, and regions predicted by NetMHCII 2.2 for binding to HLA-DRB1*09-DQB1*03:09 allele are shaded. Sites recognized by neutralizing antibodies are underlined. Sites considered for amino acid modification are marked with an asterisk above. Additionally, FIG. 1B provides an example of a network of interacting hydrophobic amino acids including two of allele binding sites, 74 and 102. Modification to 74 and/or 102 was then compensated with a suitable substitution at other positions in this network.

### Modification of Residues in Predicted Allele Binding Regions

In order to preserve the bioactivity of the EPO variants, modifications to the predicted allele binding regions were designed to reduce the human leukocyte antigen (HLA) and neutralizing antibody binding, while avoiding modification of residues involved in the EPO-R binding interface or structural integrity of the EPO polypeptide, such as residues located within the highly networked helices (B-C-D) of the alpha-helical bundle (FIG. 1B). Specifically, regions 102-116 and 142-153, that overlap with or are in proximity to known neutralizing antibody sites were selected, involving a total of 25 sites: 74, 75, 76, 77, 79, 80, 81, 82, 102, 105, 106, 109, 111, 112, 113, 114, 115, 116, 142, 145, 146, 148, 149, 152, and 153 (FIG. 1A). The non-receptor-binding site (non-RBS) residues were selected for modification, and interatomic contacts were optimized using a variant of the EPCN method (Robinson et al. Cell (2015) 162:493-504). Seventeen EPO variants with HLA-affinity-disrupting designs were devised, as shown in Table 2.

### Example 2: Expression and Purification of EPO Variants

The EPO variants designed in Example 1 and displayed in Table 2 were then recombinantly expressed and purified using the following procedures.

### Plasmid Generation and Preparation

A pcDNA 3.3 expression vector containing a sequence encoding an EPO variant having only 1 mutation/substitution site was generated using QuikChange II XL Site-Directed Mutagenesis Kit (Agilent Technologies). A sequence encoding for an EPO variant having more than 1 mutation/substitution sites was generated using gene synthesis, cloned into pcDNA 3.3 expression vector, and transformed into E. coli (DNA 2.0). Purified plasmids were submitted for DNA sequencing (Genewiz) to confirm the mutations.

### Protein Expression and Purification

The purified pcDNA 3.3 expression vector containing a sequence encoding EPO wild-type (EPO-WT), or an EPO variant, was transiently transfected into FreeStyle™ 293-F cells using 25 kD linear polyethylenimine (Polysciences). After 6 days, EPO polypeptide was purified from the culture supernatant using Hitrap Blue HP column (GE Healthcare), eluted with 1.5 M NaCl, and buffer exchanged into 20 mM Tris-HCl (pH 8.45). Next, sample was loaded onto Hitrap Q HP column (GE Healthcare), and the column was washed with 20 mM sodium acetate (pH 4), followed by a second wash with 20 mM Tris. The EPO polypeptide was then eluted with 1M NaCl, and the purified EPO polypeptide was buffer exchanged into a storage buffer (50 mM sodium phosphate buffer, pH 7, 1.5% glycine and 0.003% Tween-20).

Cell Cultures: FREESTYLE™ 293-F cells (Invitrogen) were cultivated at 37° C. under 8% CO 2 in FreeStyle 293 Expression Medium (Invitrogen) using a shaker at 130 RPM. Human erythroleukemic cell line, TF-1 (ATCC) were cultivated at 37° C. under 5% CO 2 in RPMI 1640 medium (Thermo Fisher Scientific) supplemented with 10% FBS, 1% Pen-strep and 2 ng/mL GM-CSF.

### Example 3: Characterization of EPO Variants

The EPO variants obtained in Example 2 were then quantitated and characterized according to the following procedures.

### Quantitation of EPO Polypeptides

Sandwich ELISA was developed for quantitation of both EPO wild type and EPO variant proteins. A pre-absorbed Maxisorp 96-well plate (Nunc) was coated with 2.5 μg/mL of capture antibody (mouse monoclonal IgG 2A to human EPO, MAB 2871; R&D Systems) in phosphate-buffered saline (PBS) and incubated at 4° C. overnight. The plate was washed three times with PBS, and a blocking solution of 1% BSA in PBS and 0.05% Tween (PBST) was added. The plate was incubated at room temperature for 1 hour and washed with PB ST. A biological reference preparation (BRP) of EPO batch 4 (European Directorate for the Quality of Medicines & Health Care European Pharmacopoeia, France; Burns et al. vide supra) was used to generate a standard curve. Serial dilution (2.5-fold) of the EPO standard or EPO samples was achieved with the starting concentration of 10,000 International Unit (IU) or undiluted concentration, respectively. Dilutions were performed in PBST and in duplicate. Both BRP and the samples were incubated at room temperature for 2 hours, and then washed with PBST. The primary antibody (rabbit polyclonal IgG to human EPO, AB-286-NA; R&D Systems) at a concentration of 1 μg/mL in PBST was added, and the plate was incubated at room temperature for 1 hour and washed with PBST. Subsequently, 0.1 μg/mL of secondary antibody (goat polyclonal secondary Ab to Rabbit IgG- H&L (HRP), pre-absorbed, ab97080; Abcam) in PBST was added, and the plate was incubated at room temperature for 1 hour and then washed with PBST. For detection, TMB substrate (SeraCare) was added. Quenching was achieved by adding 1N sulfuric acid. The plate was read using a microplate reader (Biotek) at 450 nm.

### Characterization of EPO Polypeptides

The protein content in the purified EPO polypeptides from FPLC was determined using SDS-PAGE together with Coomassie blue staining. Protein samples were separated using 12% sodium dodecyl sulfate (SDS) polyacrylamide gel, and then separated according to their molecular mass at a constant 175 voltage for 1.5 hours. The electrophoresed gel was stained with INSTANTBLUE™ protein stain (Expedeon). The purity of protein bands was analyzed by measurement of band intensity in each lane using the ImageJ program (Schneider et al. Nat. Methods (2012) 9:671-675). The purity of the purified proteins was 94-99%. The size of deglycosylated and glycosylated EPO polypeptide is estimated to be 30.6 kDa and 36-40 kDa, respectively. Both BRP, purified EPO-WT, and EPO variants showed expected molecular weights.

Western blot analysis using anti EPO antibody was then carried out to confirm the identity of the proteins. The proteins in the SDS-PAGE gel were transferred onto nitrocellulose membrane (Bio-Rad), and the membrane was incubated with a rabbit polyclonal IgG to human EPO, AB-286-NA (R&D Systems) followed by a donkey derived ECL™ anti-rabbit IgG, peroxidase-linked species-specific whole antibody, NA934V (GE Healthcare). The signal was developed using an ECL™ Prime Western Blotting Detection Reagent (GE Healthcare). The chemiluminescent signal was detected and analyzed using an ImageQuant LAS4000 machine (GE Healthcare). The BRP, EPO-WT and EPO variant proteins all showed positive bands.

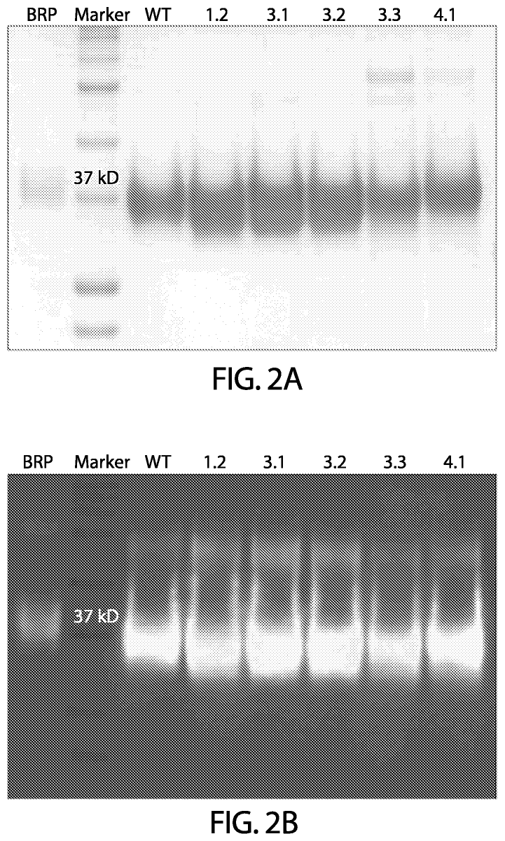

For example, FIGS. 2A-2B provide the Coomassie blue stained gel (FIG. 2A) and Western immunoblotting (FIG. 2B) of the EPO polypeptides, including EPO standard, wild type and mutant proteins, following separation according to their molecular mass with SDS-PAGE. Materials used included BRP, EPO standard; Marker, Precision Plus Protein™ Kaleidoscope™ Prestained Protein Standards; WT, EPO-WT; 1.2, EPO-1.2 (L70V, V74L, L102I); 3.1, EPO-3.1 (T106A); 3.2, EPO-3.2 (T106G); 3.3, EPO-3.3 (T106H); 4.1, EPO-4.1 (L109A).

### Characterization of EPO Isoforms

The isoform distribution of the EPO polypeptides was determined according to the TD2014EPO technical document outlined by the World Anti-Doping Agency (www.wada-ama.org/en/resources/science-medicine/td2014epo). The purified EPO samples prepared by the methods outlined above were buffer exchanged into MilliQ water and separated on an isoelectric focusing (IEF) gel according to their pI, with a pH gradient of 2-6, using a flat-bed electrophoresis cell-MultiphorII (GE Healthcare). The electrophoresed samples were then transferred to Armersham Hybond™—P PVDF transfer membrane (GE Healthcare) using a semi-dry Novablot transferring system (GE Healthcare). After blotting, the membrane was processed by Western blot analysis as described herein.

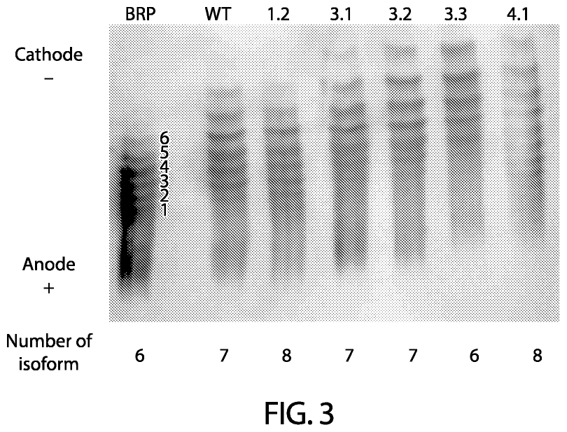

The isoform analysis by IEF of the EPO polypeptides is shown in FIG. 3, including EPO standard, wild type and mutant proteins. The separated bands of the proteins represent the number of their existing glycoforms. Distribution of different isoforms of the purified EPO polypeptide was compared to BRP, an EPO standard. BRP, EPO standard; WT, EPO-WT; 1.2, EPO-1.2 (L70V, V74L, L102I); 3.1, EPO-3.1 (T106A); 3.2, EPO-3.2 (T106G); 3.3, EPO-3.3 (T106H); 4.1, EPO-4.1 (L109A).

The glycoform distribution among BRP and 6 EPO samples (1 wild type and 5 mutants) were observed in the basic area of the gel (FIG. 3). BRP showed 6 isoforms as expected. However, the number of isoforms among EPO samples varied (EPO-3.3 also showed 6 isoforms, EPO-WT, EPO-3.1 and EPO-3.2 showed 7 isoforms while EPO-1.2 and EPO-4.1 showed 8 isoforms). EPO-3.1, EPO-3.2, EPO-3.3 and EPO-4.1 showed the shift of the isoform distribution toward the basic isoforms. The two most basic bands of those EPO samples were significantly higher than the EPO-WT. While EPO-1.2 showed comparable profile or a slight shift toward the acidic isoforms.

### Example 4: In Vitro Bioactivity of EPO Variants

The bioactivities of EPO variant proteins obtained in Example 2 (and shown in Table 2) were measured in a cell proliferation assay using a TF-1 cell line derived from a patient with erythroleukemia, according to the following procedure. A total of 2.5×10⁴ TF-1 cells were plated into a 96-well flat-bottom plate (Nunc). BRP or EPO polypeptide (50 IU/mL) was 3-fold diluted. All dilutions were done in quadruplicate. After 72 hours incubation, WST-1 Cell Proliferation Reagent (Roche) was added. The reaction was incubated in 5% CO 2 for 4 hours. The absorbance of 450 nm and 690 nm were measured using a microplate reader (Biotek). GEN5™ data analysis software (Biotek) was used to calculate EC 50 and R² using a 4-parameter logistic regression function. Parallelism analysis was performed for evaluation of relative potency (REP) of EPO variant against EPO-WT. The log dose-response curve of a 4-parameter logistic regression was generated by GraphPad Prism 6 (Graphpad Software, Inc.).

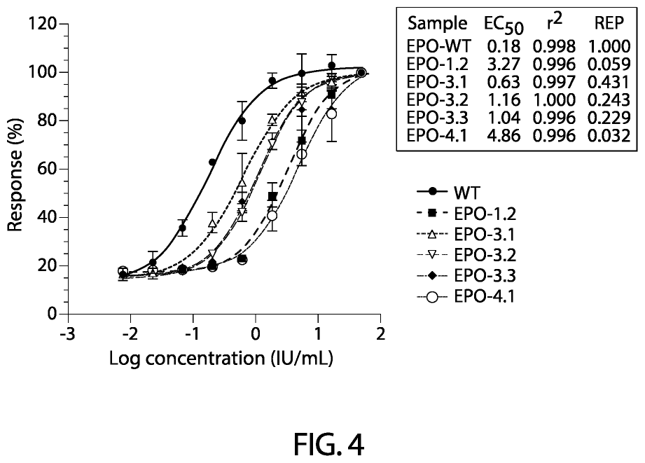

The in vitro bioactivities of selected EPO variants with TF-1 cells, including EPO-1.2 (L70V, V74L and L10231), EPO-3.1 (T106A), EPO-3.2 (T106G), EPO-3.3 (T106H), and EPO-4.1 (L109A), are displayed in the log-dose response curve in FIG. 4. All five EPO variants showed a lower bioactivity relative to wild-type EPO. In particular, EPO-1.2 and EPO-4.1 had a relative bioactivity (REP) to EPO-WT of 0.059 (or 5.9% activity compared to EPO-WT) and 0.032 (or 3.2% activity compared to EPO-WT), respectively. EPO-3.1 had the highest bioactivity among EPO variants with REP of 0.431 (or 43.1% activity compared to EPO-WT). EPO-3.2 and EPO-3.3 were in the same range of bioactivity with REP of 0.243 (or 24.3% activity compared to EPO-WT) and 0.229 (or 22.9% activity compared to EPO-WT), respectively.

### Example 5: Immunogenicity of EPO Variants

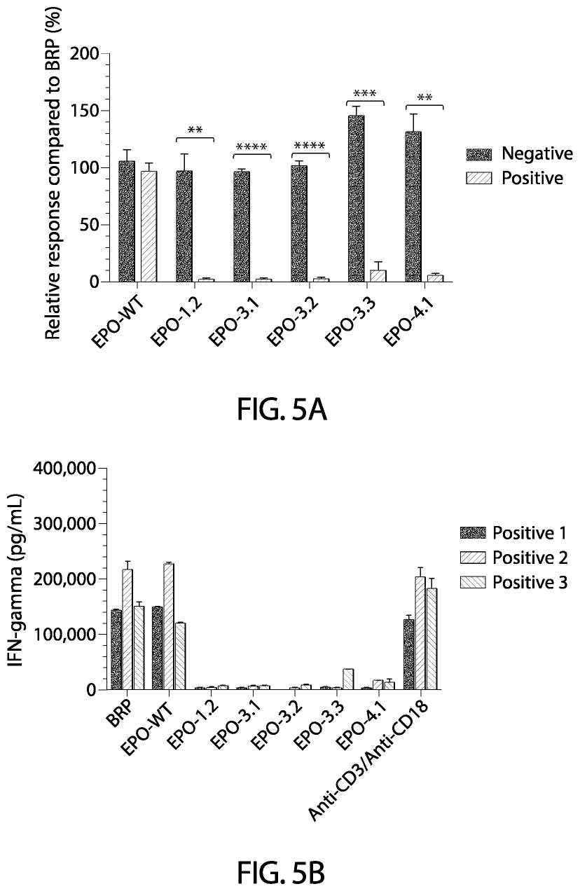

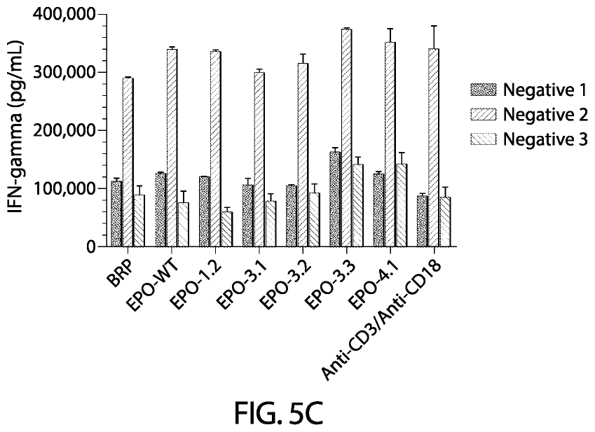

The immunogenicity of exemplary EPO variants prepared by the methods described herein (EPO-WT, EPO-1.2, EPO-3.1, EPO-3.2, EPO-3.3 and EPO-4.1) was tested by the following procedures. As illustrated by FIGS. 5A-5C, all five EPO variants that were tested exhibited lower immunogenicity relative to BRP and wild-type EPO.

### Preparation of Peripheral Blood Mononuclear Cells (PBMCs)

Blood was collected from 50 healthy volunteers in EDTA-treated vacuum tubes (Becton Dickinson). The presence of the HLA-DRB1*09 gene and common infectious diseases including hepatitis B, hepatitis C, syphilis and HIV was determined at the Faculty of Medicine, Chulalongkorn University, Thailand. Eleven out of fifty volunteers (22%) had HLA-DRB1*09, of which there were four women (36%) and seven men (64%). All HLA-DRB1*09 negative and positive volunteers were randomly selected for immunogenicity test and were confirmed to be negative for common infectious diseases including hepatitis B, hepatitis C, syphilis, and HIV. PBMCs were isolated from the blood of three HLA-DRB1*09 negative volunteers and three HLA-DRB1*09 positive volunteers using IsoPrep solution (Robbins Scientific Corporation).

### Dendritic Cell Culture and Flow Cytometric Analysis

A total of 10×10⁶ cells of PBMCs in complete RPMI medium (3 mL) were plated in each well of a 6-well plate. After incubation at 37° C. under 5% CO 2 for 2 hours, precursor dendritic cells (DCs) were separated from other immune cells by adherence to plastic, and non-adherent cells were collected as a source of CD4⁺ T cells. The complete RPMI medium containing GM-CSF (50 ng/mL) and IL-4 (50 ng/mL; ImmunoTools) was added to the adherent cells representing the precursor dendritic cells (DCs). Cells were incubated and 75% spent medium was exchanged to fresh medium with cytokines every second day. The differentiation of precursor DCs to immature DCs was monitored by a BD FACSCANTO™ Flow cytometer (BD Biosciences) using FITC-conjugated anti-human HLA-DR (ImmunoTools). The percentage of immature DC maturation of both groups ranged from 50.7 to 80.4 on day 7 of DC culture.

To generate mature DCs as antigen presenting cells (APCs), the immature DCs were harvested and washed with complete RPMI medium. A total of 10⁵ cells were then plated in each well of a 24-well plate and incubated (pulsed) with 10 μg/mL BRP or EPO polypeptide (EPO-WT, EPO-1.2, EPO-3.1, EPO-3.2, EPO-3.3 and EPO-4.1) for 48 hours Immuture DCs incubated without protein were used as control DCs (unpulsed DCs).

### Separation and Activation of CD4⁺ T Cells

CD4⁺ T cells were isolated from the nonadherent cell fraction of PBMCs from the same volunteer who provided monocytes for the DC culture, by negative selection using MACS Human CD4⁺ T cell Isolation Kit (Miltenyi Biotec). Viability was greater than 90%. To activate effector immune cells, EPO-pulsed (mature) DCs and CD4⁺ T cells derived from the same volunteer were cocultured at a 1:20 ratio. Positive control included the coculture of unpulsed DC and CD4⁺ T cells supplemented with 5 μg/mL anti-human CD3 and 2 μg/mL anti-human CD28 (ImmunoTools). Background control included the coculture of unpulsed DC and CD4⁺ T cells alone. All cocultures were incubated in the presence of IL-2 (30 ng/mL) and IL-12 (30 ng/mL) ImmunoTools) for 4 days and then harvested.

The level of interferon gamma (IFN-γ) representing T cell response was quantified by human IFN-γ ELISA Kit (ImmunoTools). The level of IFN-γ from coculture between unpulsed DC and CD4⁺ T cells was considered as a background of T cell response. Statistical comparison of the relative T cell response of EPO polypeptide compared to BRP between HLA-DRB1*09-negative and positive groups demonstrated that all five EPO variants tested had a significantly lower T cell response in the HLA-DRB1*09-positive group.

The results of the ex vivo immunogenicity assay of EPO polypeptides are displayed in FIGS. 5A-5C. The mean value from 3 HLA-DRB1*09-negative volunteers and 3 HLA-DRB1*09-positive volunteers were combined as a negative group or a positive group, respectively. Each group was relatively compared to the response to BRP and presented in the percentage of relative response of EPO polypeptides (FIG. 5A). Bar graphs are shown as mean±SEM calculated from 3 individuals in each group. Asterisks denote a significant difference by t-test (*p<0.05, **p<0.01, ***p<0.001, ****p<0.0001). IFN-γ level of each HLA-DRB1*09-positive volunteers is shown in FIG. 5B, and HLA-DRB1*09-negative volunteers shown in FIG. 5C. Bar graphs are shown as mean±SEM calculated from duplicate experiments.

The p-values were 0.0038 for EPO-1.2, 0.000024 for EPO-3.1, 0.000037 for EPO-3.2, 0.00031 for EPO-3.3 and 0.0012 for EPO-4.1. There was no difference in T cell response using EPO-WT in both HLA-DRB1*09-negative and positive groups. In both HLA-DRB1*09-positive and negative group, BRP and EPO-WT could stimulate T cell response in the same manner as anti-CD3/anti-CD28 antibodies. EPO variants including EPO-1.2, EPO-3.1, EPO-3.2, EPO-3.3 and EPO-4.1 exhibited a much lower T cell response with the IFN-γ release ranging from 220 to 37,000 pg/mL. Without wishing to be bound by theory, the different background of EPO-specific T cell response seen in HLA-DRB1*09-positive and negative group can be due to the difference in HLA allele distribution in individuals (see, e.g., Praditpornsilpa et al. vide supra; and Tangri et al. J. Immunol. (2005) 174:3187-3196).

Statistical Analysis: T-test analyses were carried out using GraphPad Prism 6 (Graphpad Software, Inc.). For example, a t-test was used to determine the statistical significance of differences in the relative T cell response between HLA-DRB1*09-negative and positive groups.

Human Subjects: Research involving human subjects was performed under a protocol approved by the ethical review committee, Chulabhorn Research Institute, Thailand. All volunteers gave a written informed consent.

### INCORPORATION BY REFERENCE

All publications, patents, and accession numbers mentioned herein are hereby incorporated by reference in their entirety as if each individual publication or patent was specifically and individually indicated to be incorporated by reference.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

What is claimed is:

1. An EPO molecule comprising a mutation at a position corresponding to any of positions 12-152 of SEQ ID NO: 1.

2. The EPO molecule of claim 1, comprising a mutation at a position corresponding to any of positions 74-82, 102-116, or 142-153 of SEQ ID NO: 1.

3. The EPO molecule of claim 1 or 2, comprising a mutation at a position corresponding to any of positions 74-82 or 102-116 of SEQ ID NO: 1.

4. The EPO molecule of any of claims 1-3, comprising a mutation at a position corresponding to any of positions 74-82 or 142-153 of SEQ ID NO: 1.

5. The EPO molecule of any of claims 1-4, comprising a mutation at a position corresponding to any of positions 102-116 or 142-153 of SEQ ID NO: 1.

6. The EPO molecule of any of claims 1-5, comprising a mutation at a position corresponding to any of positions 74-82 of SEQ ID NO: 1.

7. The EPO molecule of any of claims 1-6, comprising a mutation at a position corresponding to any of positions 102-116 of SEQ ID NO: 1.

8. The EPO molecule of any of claims 1-7, comprising a mutation at a position corresponding to any of positions 102-110 of SEQ ID NO: 1.

9. The EPO molecule of any of claims 1-8, comprising a mutation at a position corresponding to any of positions 108-116 of SEQ ID NO: 1.

10. The EPO molecule of any of claims 1-9, comprising a mutation at a position corresponding to any of positions 142-153 of SEQ ID NO: 1.

11. The EPO molecule of any of claims 1-10, comprising a mutation at a position corresponding to any of positions 142-150 of SEQ ID NO: 1.

12. The EPO molecule of any of claims 1-11, comprising a mutation at a position corresponding to any of positions 145-153 of SEQ ID NO: 1.

13. The EPO molecule of any of claims 1-12, comprising a mutation at a position corresponding to any of positions 74, 75, 76, 77, 79, 80, 81, 82, 102, 105, 106, 109, 111, 112, 113, 114, 115, 116, 142, 145, 146, 148, 149, 152, or 153 of SEQ ID NO: 1.

14. The EPO molecule of any of claims 1-13, comprising a mutation at a position corresponding to any of positions 12, 15, 67, 70, 74, 102, 105, 106, 109, 114, 146, 149, or 152 of SEQ ID NO: 1.

15. The EPO molecule of any of claims 1-14, comprising a plurality of mutations at a plurality of positions.

16. The EPO molecule of claim 15, further comprising a second mutation at a second position disclosed herein.

17. The EPO molecule of claim 16, further comprising a third mutation at a third position disclosed herein.

18. The EPO molecule of claim 17, further comprising a fourth mutation at a fourth position disclosed herein.

19. The EPO molecule of claim 18, further comprising a fifth mutation at a fifth position disclosed herein.

20. The EPO molecule of claim 19, further comprising a sixth mutation at a sixth position disclosed herein.

21. The EPO molecule of any of claims 1-20, comprising a mutation at a position corresponding to position 12 of SEQ ID NO: 1.

22. The EPO molecule of claim 21, wherein the mutation is an L12A substitution.

23. The EPO molecule of claim 21, wherein the mutation is an L12I substitution.

24. The EPO molecule of any of claims 1-23, comprising a mutation at a position corresponding to position 15 of SEQ ID NO: 1.

25. The EPO molecule of claim 24, wherein the mutation is a Y15I substitution.

26. The EPO molecule of claim 24, wherein the mutation is a Y15W substitution.

27. The EPO molecule of any of claims 1-26, comprising a mutation at a position corresponding to position 67 of SEQ ID NO: 1.

28. The EPO molecule of claim 27, wherein the mutation is a L67D substitution.

29. The EPO molecule of claim 27, wherein the mutation is a L67N substitution.

30. The EPO molecule of claim 27, wherein the mutation is a L67V substitution.

31. The EPO molecule of any of claims 1-30, comprising a mutation at a position corresponding to position 70 of SEQ ID NO: 1.

32. The EPO molecule of claim 31, wherein the mutation is an L70V substitution.

33. The EPO molecule of any of claims 1-32, comprising a mutation at a position corresponding to position 74 of SEQ ID NO: 1.

34. The EPO molecule of claim 33, wherein the mutation is a V74D substitution.

35. The EPO molecule of claim 33, wherein the mutation is a V74L substitution.

36. The EPO molecule of claim 33, wherein the mutation is a V74N substitution.

37. The EPO molecule of any of claims 1-36, comprising a mutation at a position corresponding to position 102 of SEQ ID NO: 1.

38. The EPO molecule of claim 37, wherein the mutation is an L102D substitution.

39. The EPO molecule of claim 37, wherein the mutation is an L102N substitution.

40. The EPO molecule of claim 37, wherein the mutation is an L1021 substitution.

41. The EPO molecule of claim 37, wherein the mutation is an L102T substitution.

42. The EPO molecule of claim 37, wherein the mutation is an L102V substitution.

43. The EPO molecule of any of claims 1-42, comprising a mutation at a position corresponding to position 105 of SEQ ID NO: 1.

44. The EPO molecule of claim 43, wherein the mutation is an L105F substitution.

45. The EPO molecule of claim 43, wherein the mutation is an L105S substitution.

46. The EPO molecule of any of claims 1-45, comprising a mutation at a position corresponding to position 106 of SEQ ID NO: 1.

47. The EPO molecule of claim 46, wherein the mutation is a T106A substitution.

48. The EPO molecule of claim 46, wherein the mutation is a T106G substitution.

49. The EPO molecule of claim 46, wherein the mutation is a T106H substitution.

50. The EPO molecule of any of claims 1-49, comprising a mutation at a position corresponding to position 109 of SEQ ID NO: 1.

51. The EPO molecule of claim 50, wherein the mutation is an L109A substitution.

52. The EPO molecule of claim 50, wherein the mutation is an L109C substitution.

53. The EPO molecule of claim 50, wherein the mutation is an L109W substitution.

54. The EPO molecule of any of claims 1-53, comprising a mutation at a position corresponding to position 114 of SEQ ID NO: 1.

55. The EPO molecule of claim 54, wherein the mutation is an A114C substitution.

56. The EPO molecule of any of claims 1-55, comprising a mutation at a position corresponding to position 146 of SEQ ID NO: 1.

57. The EPO molecule of claim 56, wherein the mutation is an S146N substitution.

58. The EPO molecule of any of claims 1-57, comprising a mutation at a position corresponding to position 149 of SEQ ID NO: 1.

59. The EPO molecule of claim 58, wherein the mutation is an L149V substitution.

60. The EPO molecule of claim 58, wherein the mutation is an L149W substitution.

61. The EPO molecule of any of claims 1-60, comprising a mutation at a position corresponding to position 152 of SEQ ID NO: 1.

62. The EPO molecule of claim 61, wherein the mutation is a K152Q substitution.

63. The EPO molecule of any of claims 1-62, comprising mutations at positions corresponding to positions 67 and 102 of SEQ ID NO: 1.

64. The EPO molecule of claim 63, wherein the mutations are L67V and L102V substitutions.

65. The EPO molecule of any of claims 1-64, comprising mutations at positions corresponding to positions 70, 74, and 102.

66. The EPO molecule of claim 65, wherein the mutations are L70V, V74L, and L1021 substitutions.

67. The EPO molecule of any of claims 1-66, comprising mutations at positions corresponding to positions 67, 74, and 102.

68. The EPO molecule of claim 67, wherein the mutations are L67N, V74N, and L102D substitutions.

69. The EPO molecule of claim 67, wherein the mutations are L67D, V74D, and L102N substitutions.

70. The EPO molecule of any of claims 1-69, comprising mutations at positions corresponding to positions 12, 15, 105, and 149.

71. The EPO molecule of claim 70, wherein the mutations are L12A, Y151, L105F, and L149W substitutions.

72. The EPO molecule of claim 70, wherein the mutations are L121, Y15W, L105S, and L149V substitutions.

73. The EPO molecule of any of claims 1-72, comprising mutations at positions corresponding to positions 109 and 114.

74. The EPO molecule of claim 73, wherein the mutations are L109C and A114C substitutions.

75. The EPO molecule of any of claims 1-74, comprising mutations at positions corresponding to positions 102, 105, 106, and 109.

76. The EPO molecule of claim 75, wherein the mutations are L102T, L105S, T106H, and L109W substitutions.

77. The EPO molecule of claim 75, wherein the mutations are L102T, L105S, T106G, and L109W substitutions.

78. The EPO molecule of any of claims 1-77, comprising mutations at positions corresponding to positions 146 and 152.

79. The EPO molecule of claim 78, wherein the mutations are S146N and K152Q substitutions.

80. The EPO molecule of any of claims 1-79, having at least 80% homology with the amino acid sequence of SEQ ID NO: 1.

81. The EPO molecule of any of claims 1-80, having at least 85% homology with the amino acid sequence of SEQ ID NO: 1.

82. The EPO molecule of any of claims 1-81, having at least 90% homology with the amino acid sequence of SEQ ID NO: 1.

83. The EPO molecule of any of claims 1-82, having at least 95% homology with the amino acid sequence of SEQ ID NO: 1.

84. The EPO molecule of any of claims 1-83, having at least 96% homology with the amino acid sequence of SEQ ID NO: 1.

85. The EPO molecule of any of claims 1-84, having at least 97% homology with the amino acid sequence of SEQ ID NO: 1.

86. The EPO molecule of any of claims 1-85, having at least 98% homology with the amino acid sequence of SEQ ID NO: 1.

87. The EPO molecule of any of claims 1-86, having at least 99% homology with the amino acid sequence of SEQ ID NO: 1.

88. The EPO molecule of any of claims 1-87, which, other than the mutation(s) at the position(s) disclosed herein, differs at no more than 15 positions with the amino acid sequence of SEQ ID NO: 1.

89. The EPO molecule of any of claims 1-88, which, other than the mutation(s) at the position(s) disclosed herein, differs at no more than 10 positions with the amino acid sequence of SEQ ID NO: 1.

90. The EPO molecule of any of claims 1-89, which, other than the mutation(s) at the position(s) disclosed herein, differs at no more than 5 positions with the amino acid sequence of SEQ ID NO: 1.

91. The EPO molecule of any of claims 1-90, which, other than the mutation(s) at the position(s) disclosed herein, differs at no more than 4 positions with the amino acid sequence of SEQ ID NO: 1.

92. The EPO molecule of any of claims 1-91, which, other than the mutation(s) at the position(s) disclosed herein, differs at no more than 3 positions with the amino acid sequence of SEQ ID NO: 1.

93. The EPO molecule of any of claims 1-92, which, other than the mutation(s) at the position(s) disclosed herein, differs at no more than 2 positions with the amino acid sequence of SEQ ID NO: 1.

94. The EPO molecule of any of claims 1-93, which, other than the mutation(s) at the position(s) disclosed herein, differs at no more than 1 position with the amino acid sequence of SEQ ID NO: 1.

95. The EPO molecule of any of claims 1-94, which differs at no more than 15 positions with the amino acid sequence of SEQ ID NO: 1.

96. The EPO molecule of any of claims 1-95, which differs at no more than 10 positions with the amino acid sequence of SEQ ID NO: 1.

97. The EPO molecule of any of claims 1-96, which differs at no more than 5 positions with the amino acid sequence of SEQ ID NO: 1.

98. The EPO molecule of any of claims 1-97, which differs at no more than 4 positions with the amino acid sequence of SEQ ID NO: 1.

99. The EPO molecule of any of claims 1-98, which differs at no more than 3 positions with the amino acid sequence of SEQ ID NO: 1.

100. The EPO molecule of any of claims 1-99, which differs at no more than 2 positions with the amino acid sequence of SEQ ID NO: 1.

101. The EPO molecule of any of claims 1-100, which differs at no more than 1 position with the amino acid sequence of SEQ ID NO: 1.

102. The EPO molecule of any of claims 1-101, comprising the amino acid sequence of any of SEQ ID NOs: 3, 5, 7, 9, 11, 13, 15, 17, 19, 21, 23, 25, 27, 29, 31, 33, or 35, or an amino acid sequence that is at least 90%, 91%, 92%, 93%, 94%, 95%, 96%, 97%, 98%, or 99% identical thereto, or differs by no more than 1, 2, 3, 4, 5, 6, 7. 8, 9, or 10 amino acids therefrom.

103. The EPO molecule of any of claims 1-102, comprising the amino acid sequence of any of SEQ ID NOs: 3, 5, 7, 9, 11, 13, 15, 17, 19, 21, 23, 25, 27, 29, 31, 33, or 35.

104. The EPO molecule of any of claims 1-103, comprising or consisting of the amino acid sequence of SEQ ID NO: 3.

105. The EPO molecule of any of claims 1-103, comprising or consisting of the amino acid sequence of SEQ ID NO: 5.

106. The EPO molecule of any of claims 1-103, comprising or consisting of the amino acid sequence of SEQ ID NO: 7.

107. The EPO molecule of any of claims 1-103, comprising or consisting of the amino acid sequence of SEQ ID NO: 9.

108. The EPO molecule of any of claims 1-103, comprising or consisting of the amino acid sequence of SEQ ID NO: 11.

109. The EPO molecule of any of claims 1-103, comprising or consisting of the amino acid sequence of SEQ ID NO: 13.

110. The EPO molecule of any of claims 1-103, comprising or consisting of the amino acid sequence of SEQ ID NO: 15.

111. The EPO molecule of any of claims 1-103, comprising or consisting of the amino acid sequence of SEQ ID NO: 17.

112. The EPO molecule of any of claims 1-103, comprising or consisting of the amino acid sequence of SEQ ID NO: 19.

113. The EPO molecule of any of claims 1-103, comprising or consisting of the amino acid sequence of SEQ ID NO: 21.

114. The EPO molecule of any of claims 1-103, comprising or consisting of the amino acid sequence of SEQ ID NO: 23.

115. The EPO molecule of any of claims 1-103, comprising or consisting of the amino acid sequence of SEQ ID NO: 25.

116. The EPO molecule of any of claims 1-103, comprising or consisting of the amino acid sequence of SEQ ID NO: 27.

117. The EPO molecule of any of claims 1-103, comprising or consisting of the amino acid sequence of SEQ ID NO: 29.

118. The EPO molecule of any of claims 1-103, comprising or consisting of the amino acid sequence of SEQ ID NO: 31.

119. The EPO molecule of any of claims 1-103, comprising or consisting of the amino acid sequence of SEQ ID NO: 33.

120. The EPO molecule of any of claims 1-103, comprising or consisting of the amino acid sequence of SEQ ID NO: 35.

121. The EPO molecule of any of claims 1-120, having a reduced immunogenicity in a subject, as compared to a reference EPO molecule, e.g., an EPO molecule comprising the amino acid sequence of SEQ ID NO: 1, e.g., as determined in vitro, ex vivo, or in vivo.

122. The EPO molecule of claim 121, wherein the immunogenicity is reduced by at least 10%, 20%, 30%, 40%, 50%, 60%, 70%, 80%, or 90%, as compared to the reference EPO molecule.

123. The EPO molecule of claim 121 or 122, wherein the subject carries the HLA-DRB1*09 allele.

124. The EPO molecule of any of claims 1-123, which has the same, or substantially the same, biological activity, as compared to a reference EPO molecule, e.g., an EPO molecule comprising the amino acid sequence of SEQ ID NO: 1, e.g., as determined in vitro, ex vivo, or in vivo.

125. The EPO molecule of any of claims 1-124, which has a biological activity that is reduced by no more than 25%, 20%, 15%, 10%, 5%, or 2%, as compared to a reference EPO molecule, e.g., an EPO molecule comprising the amino acid sequence of SEQ ID NO: 1, e.g., as determined in vitro, ex vivo, or in vivo.

126. The EPO molecule of any of claims 124 or 125, wherein the biological activity is determined in an in vitro TF-1 proliferation assay.

127. The EPO molecule of any of claims 1-126, which is capable of having more glycoforms, e.g., at least 1, 2, 3, 4, or 5 more glycoforms, as compared to a reference EPO molecule, e.g., an EPO molecule comprising the amino acid sequence of SEQ ID NO: 1.

128. The EPO molecule of any of claims 1-127, which is capable of having 6, 7, 8, 9, 10, or more glycoforms.

129. The EPO molecule of any of claims 1-128, which is capable of having 7, 8, or more glycoforms.

130. The EPO molecule of any of claims 1-129, which has the same, or substantially the same, serum half-life, as compared to a reference EPO molecule, e.g., an EPO molecule comprising the amino acid sequence of SEQ ID NO: 1.

131. The EPO molecule of any of claims 1-130, which has an increased serum half-life, e.g., increased by at least 10%, 20%, 30%, 40%, 50%, 60%, 70%, 80%, or 90%, or at least 1, 2, 3, 4, 5, 6, 7, 8, 9, or 10-fold, as compared to a reference EPO molecule, e.g., an EPO molecule comprising the amino acid sequence of SEQ ID NO: 1.

132. The EPO molecule of any of claims 1-131, which has a reduced serum half-life, e.g., reduced by no more than 10%, 20%, 30%, 40%, 50%, 60%, 70%, 80%, or 90%, as compared to a reference EPO molecule, e.g., an EPO molecule comprising the amino acid sequence of SEQ ID NO: 1.

133. The EPO molecule of any of claims 1-132, which comprises a moiety that extends the serum half-life of an EPO polypeptide.

134. The EPO molecule of any of claims 1-133, which is a fusion protein.

135. The EPO molecule of any of claims 1-134, which comprises an Fc domain of an IgG.

136. The EPO molecule of claim 135, wherein the Fc domain covalently fused to an EPO polypeptide.

137. The EPO molecule of claim 136, wherein the N-terminus of the Fc domain is covalently fused to the C-terminus of the EPO polypeptide directly or indirectly.

138. A pharmaceutical composition comprising the EPO molecule of any of claims 1-137 and a pharmaceutically acceptable carrier.

139. A preparation comprising the EPO molecule of any of claims 1-137.

140. The preparation of claim 139, which is suitable for therapeutic use.

141. A kit comprising the EPO molecule of any of claims 1-137 and instructions for use.

142. A nucleic acid encoding the EPO molecule of any of claims 1-137.

143. A nucleic acid comprising the nucleotide sequence of any of SEQ ID NOs: 4, 6, 8, 10, 12, 14, 16, 18, 20, 22, 24, 26, 28, 30, 32, 34, or 36, or a nucleotide sequence that differs by no more than 5, 10, 15, 20, 25, 30, 35, 40, 45, or 50 nucleotides therefrom, or is at least 80%, 85%, 90%, 95%, 98%, or 99% identical thereto.

144. The nucleic acid of claim 143, comprising the nucleotide sequence of any of SEQ ID NOs: 4, 6, 8, 10, 12, 14, 16, 18, 20, 22, 24, 26, 28, 30, 32, 34, or 36.

145. A vector comprising the nucleic acid of any of claims 142-144.

146. The vector of claim 145, comprising a promoter operably linked to the nucleic acid.

147. A cell comprising the nucleic acid of any of claims 142-144.

148. A method of producing an EPO molecule, comprising culturing the cell of claim 147 under conditions that allows for expression of EPO polypeptide, thereby producing the EPO molecule.

149. A method of treating a disorder in a subject, comprising administering to the subject an effective amount of the EPO molecule of any of claims 1-137, thereby treating the disorder.

150. The method of claim 149, wherein the disorder is a disorder associated with EPO, e.g., a deficiency or abnormality of EPO.

151. The method of claim 150, wherein the disorder is anemia.

152. The method of claim 151, wherein the anemia is associated with or due to a chronic kidney disease (e.g., chronic renal failure), a zidovudine treatment, a chemotherapy, or a surgery.

153. A method of reducing an immunogenic response in a subject, comprising administering to a subject in need thereof an effective amount of the EPO molecule of any of claims 1-137, thereby reducing the immunogenic response.

154. A method of treating or preventing pure red-cell aplasia (PRCA) in a subject, comprising administering to a subject in need thereof an effective amount of the EPO molecule of any of claims 1-137, thereby treating or preventing PRCA.

155. A method of reducing an allogenic red blood cell (RBC) transfusion in a subject, comprising administering to a subject in need thereof an effective amount of the EPO molecule of any of claims 1-137, thereby reducing the allogenic RBC transfusion.

156. The method of any of claims 148-155, further comprising acquiring genotype information or a sequence that identifies an HLA genotype, e.g., the HLA-DRB1*09 genotype, in the subject, wherein the presence of the HLA genotype identifies the subject has, or is at risk of having, an increased immunogenicity to the EPO therapy.

157. The method of claim 156, wherein the EPO molecule is administered responsive to the acquisition of the genotype information.

158. A method of evaluating a subject, comprising acquiring genotype information that identifies an HLA genotype, e.g., the HLA-DRB1*09 genotype, in the subject, wherein the presence of the HLA genotype identifies the subject has, or is at risk of having, an increased immunogenicity to a therapy comprising an EPO molecule.

159. The method of claim 158, further comprising classifying the subject as a candidate for treatment with an EPO molecule disclosed herein.

160. The method of claim, 158 or 159, further comprising administering to the subject an effective amount of an EPO molecule disclosed herein.

161. The method of any of claims 148-160, wherein the subject carries, or has been determined to carry, the HLA-DRB1*09 allele.

162. The method of any of claims 148-161, wherein the subject has a chronic kidney disease, an HIV infection, or a cancer.

163. The method of any of claims 148-162, wherein the subject receives, or has received, zidovudine.

164. The method of any of claims 148-163, wherein the subject receives, or has received, a chemotherapy (e.g., a myelosuppressive chemotherapy), a surgery, or an allogenic RBC transfusion.

165. The method of any of claims 148-164, wherein the subject has, or has been determined to have, anti-EPO antibodies.

166. The method of any of claims 148-165, wherein the subject has been administered a different recombinant human EPO (rHuEPO), e.g., an EPO comprising the amino acid sequence of SEQ ID NO: 1.

167. The method of claim 166, wherein the administration of the different rHuEPO is discontinued or terminated.
